SEQUENCE LISTING (41 sequences)

SEQ ID NO: 1 - protein, 441 aa, synthetic construct
  REGION 1..441: note = tau protein 2N4R
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 10 aa, synthetic construct
  REGION 1..10: note = CDR-H1 Sequence of PT82 mAb
GFTFSNYWMN

SEQ ID NO: 3 - protein, 19 aa, synthetic construct
  REGION 1..19: note = CDR-H2 Sequence of PT82 mAb
QIRLQSDNYATRYAESVKG

SEQ ID NO: 4 - protein, 4 aa, synthetic construct
  REGION 1..4: note = CDR-H3 sequence of PT82 mAb
GITY

SEQ ID NO: 5 - protein, 11 aa, synthetic construct
  REGION 1..11: note = CDR-L1 Sequence of PT82 mAb
KASQNVGTAVA

SEQ ID NO: 6 - protein, 7 aa, synthetic construct
  REGION 1..7: note = CDR-L2 Sequence of PT82 mAb
SASIRYT

SEQ ID NO: 7 - protein, 9 aa, synthetic construct
  REGION 1..9: note = CDR-L3 Sequence of PT82 mAb
QQFSSYPYT

SEQ ID NO: 8 - protein, 115 aa, synthetic construct
  REGION 1..115: note = VH sequence of PT82 mAb
EVKLEESGGGLVQPGGSMKLSCVASGFTFSNYWMNWIRQSPEKGLEWVAQIRLQSDNYATRYAESVKGRFTISRDESKTSVYLQMNNLRTEDTGIYYCTGGTTYWGQGTLVTVSA

SEQ ID NO: 9 - protein, 107 aa, synthetic construct
  REGION 1..107: note = VL sequence of PT82 mAb
DIVMTQSQKFMSTSVGDRVSITCKASQNVGTAVAWYQQKPGQSPKLLIYSASIRYTGVPDRFTGSGSGTDFTLTINYMQSEDLADYFCQQFSSYPYTFGGGTKLEIK

SEQ ID NO: 10 - protein, 8 aa, synthetic construct
  REGION 1..8: note = PT82 mAb epitope, aa119-126 of SEQ ID NO:1
AGHVTQAR

SEQ ID NO: 11 - protein, 11 aa, synthetic construct
  REGION 1..11: note = PT82 mAb epitope, aa117 to 127 of SEQ ID NO:1
EAAGHVTQARM

SEQ ID NO: 12 - protein, 10 aa, synthetic construct
  REGION 1..10: note = HCDR1 of HT43 mAb
GFTFRSYGMS

SEQ ID NO: 13 - protein, 17 aa, synthetic construct
  REGION 1..17: note = HCDR2 of HT43 mAb
TINSDGSYTYYPDSVKG

SEQ ID NO: 14 - protein, 8 aa, synthetic construct
  REGION 1..8: note = HCDR3 of HT43 mAb
SWDGAMDY

SEQ ID NO: 15 - protein, 16 aa, synthetic construct
  REGION 1..16: note = LCDR1 of HT43 mAb
RSSQSILHSNGNTYFE

SEQ ID NO: 16 - protein, 7 aa, synthetic construct
  REGION 1..7: note = LCDR2 of HT43 mAb
KVSNRFS

SEQ ID NO: 17 - protein, 9 aa, synthetic construct
  REGION 1..9: note = LCDR3 of HT43 mAb
FQGSLVPWT

SEQ ID NO: 18 - protein, 117 aa, synthetic construct
  REGION 1..117: note = Heavy chain variable region of HT43 mAb
EVKLVESGGDLVKPGGSLKLSCAASGFTFRSYGMSWVRQTPDKRLEWVTTINSDGSYTYYPDSVKGRFTISRDNAKNTLYLQMSSLKSEDTAMYYCAVSWDGAMDYWGQGTSVTVSS

SEQ ID NO: 19 - protein, 112 aa, synthetic construct
  REGION 1..112: note = Light chain variable region of HT43 mAb
DVLVTQTPLSLPVSLGDQASISCRSSQSILHSNGNTYFEWYLQRPGQSPKLLIYKVSNRFSGVPDRFSGSGSGTDFTLKISRVEAEDLGVYYCFQGSLVPWTFGGGTKLEIK

SEQ ID NO: 20 - protein, 14 aa, synthetic construct
  REGION 1..14: note = HT43 mAb epitope, aa7 to 20 of SEQ ID NO:1
EFEVMEDHAGTYGL

SEQ ID NO: 21 - protein, 5 aa, synthetic construct
  REGION 1..5: note = hT7 mAb epitope, aa 159-163 of SEQ ID NO: 1
PPGQK

SEQ ID NO: 22 - protein, 20 aa, synthetic construct
  REGION 1..20: note = calibrant peptide pT3xhT43
  SITE 20: note = Residue attached to another sequence via 4x discrete polyethylene glycol (dPEG4) linker
PRQEFEVMEDHAGTYGLGDR

SEQ ID NO: 23 - protein, 18 aa, synthetic construct
  REGION 1..18: note = calibrant peptide pT3xpT82
  SITE 1: note = N-terminal acetyl group
  SITE 18: note = Residue attached to another sequence via 4x discrete polyethylene glycol (dPEG4) linker
SLEDEAAGHVTQARMVSK

SEQ ID NO: 24 - protein, 20 aa, synthetic construct
  REGION 1..20: note = calibrant peptide hT7xpT82
  SITE 1: note = N-terminal acetyl group
  SITE 20: note = Residue attached to another sequence via 4x discrete polyethylene glycol (dPEG4) linker
ASLEDEAAGHVTQARMVSKP

SEQ ID NO: 25 - protein, 11 aa, synthetic construct
  REGION 1..11: note = p217+ tau epitope
  SITE 2: note = phosphorylated threonine
RTPSLPTPPTR

SEQ ID NO: 26 - protein, 11 aa, synthetic construct
  REGION 1..11: note = p217+ tau epitope
  SITE 7: note = phosphorylated threonine
RTPSLPTPPTR

SEQ ID NO: 27 - protein, 11 aa, synthetic construct
  REGION 1..11: note = p217+ tau epitope
  SITE 2: note = phosphorylated threonine
  SITE 7: note = phosphorylated threonine
RTPSLPTPPTR

SEQ ID NO: 28 - protein, 118 aa, synthetic construct
  REGION 1..118: note = PT3 mouse mAb VH
EVKLVESGGDLVKPGGSLKLSCAASGFTFSSYAMSWVRQNPEKRLEWVASISKGGNTYYPNSVKGRFTISRDNARNILYLQMSSLRSEDTALYYCARGWGDYGWFAYWGQVTLVTVSA

SEQ ID NO: 29 - protein, 107 aa, synthetic construct
  REGION 1..107: note = PT3 mouse mAb VL
DIKMTQSPSSMYASLGERVTITCKASQDINRYLNWFQQKPGKSPKTLIYRANRLLDGVPSRFSGSGSGQDYSLTISSLDYEDMGIYYCLQYDEFPLTFGDGTKLELK

SEQ ID NO: 30 - protein, 118 aa, synthetic construct
  REGION 1..118: note = PT3 humanized mAb VH
QVQLVESGGGVVQPGRSLRLSCAASGFTFSSYAMSWVRQAPGKGLEWVASISKGGNTYYADSVKGRFTISRDNSKNTLYLQMNSLRAEDTAVYYCARGWGDYGWFAYWGQVTLVTVSS

SEQ ID NO: 31 - protein, 107 aa, synthetic construct
  REGION 1..107: note = PT3 humanized mAb VL
DIQMTQSPSSLSASVGDRVTITCKASQDINRYLNWFQQKPGKAPKSLIYRANRLLDGVPSRFSGSGSGTDFTLTISSLQPEDFATYYCLQYDEFPLTFGQGTKLEIK

SEQ ID NO: 32 - protein, 5 aa, synthetic construct
  REGION 1..5: note = PT3 mouse mAb HCDR1, Kabat numbering
SYAMS

SEQ ID NO: 33 - protein, 16 aa, synthetic construct
  REGION 1..16: note = PT3 mouse mAb HCDR2, Kabat numbering
SISKGGNTYYPNSVKG

SEQ ID NO: 34 - protein, 10 aa, synthetic construct
  REGION 1..10: note = PT3 mouse mAb HCDR3, Kabat numbering
GWGDYGWFAY

SEQ ID NO: 35 - protein, 11 aa, synthetic construct
  REGION 1..11: note = PT3 mouse mAb LCDR1, Kabat numbering
KASQDINRYLN

SEQ ID NO: 36 - protein, 7 aa, synthetic construct
  REGION 1..7: note = PT3 mouse mAb LCDR2, Kabat numbering
RANRLLD

SEQ ID NO: 37 - protein, 9 aa, synthetic construct
  REGION 1..9: note = PT3 mouse mAb LCDR3, Kabat numbering
LQYDEFPLT

SEQ ID NO: 38 - protein, 18 aa, synthetic construct
  REGION 1..18: note = calibrant peptide pT3xhT43
  SITE 1: note = Residue attached to another sequence via 4x discrete polyethylene glycol (dPEG4) linker
  SITE 18: note = Residue attached to another sequence via 4x discrete polyethylene glycol (dPEG4) linker
GKTKIATPRGAAPPGQKG

SEQ ID NO: 39 - protein, 20 aa, synthetic construct
  REGION 1..20: note = calibrant peptide pT3xhT43
  SITE 1: note = Residue attached to another sequence via 4x discrete polyethylene glycol (dPEG4) linker
  SITE 6: note = phosphorylated threonine
  SITE 11: note = phosphorylated threonine
  SITE 20: note = C-terminal amide
GSRSRTPSLPTPPTREPKKV

SEQ ID NO: 40 - protein, 20 aa, synthetic construct
  REGION 1..20: note = calibrant peptide pT3xpT82
  SITE 1: note = Residue attached to another sequence via 4x discrete polyethylene glycol (dPEG4) linker
  SITE 6: note = phosphorylated threonine
  SITE 11: note = phosphorylated threonine
  SITE 20: note = C-terminal amide
GSRSRTPSLPTPPTREPKKV

SEQ ID NO: 41 - protein, 14 aa, synthetic construct
  REGION 1..14: note = calibrant peptide hT7xpT82
  SITE 1: note = Residue attached to another sequence via 4x discrete polyethylene glycol (dPEG4) linker
  SITE 14: note = C-terminal amide
RGAAPPGQKGQANA

(19) United States
(12) Patent Application Publication
(10) Pub. No.: US 2026/0177562 A1
(43) Pub. Date: Jun. 25, 2026

(54) ASSAYS TO DETECT NEURODEGENERATION

(71) Applicant: Janssen Pharmaceutica NV, Beerse (BE)

(72) Inventors: Hartmuth Christian Kolb, San Diego, CA (US); Gallen Triana-Baltzer, San Diego, CA (US); John Randall Slemmon, West Windsor, NJ (US)

(21) Appl. No.: 19/201,475

(22) Filed: May 7, 2025

Related U.S. Application Data
(63) Continuation of application No. 16/978,006, now abandoned.
(60) Provisional application No. 62/638,524, filed on Mar. 5, 2018.

(51) Int. Cl. G01N 33/68; C12Q 1/48; G01N 1/40; G01N 30/02; G01N 33/573

(52) U.S. Cl. CPC G01N 33/6896; C12Q 1/485; G01N 1/4005; G01N 33/573; G01N 30/02; G01N 2030/027; G01N 2440/14; G01N 2800/2821; G01N 2800/2828; G01N 2800/50; G01N 2800/52; G01N 2800/56

(57) ABSTRACT

Methods of measuring the amount of singly- or multiply-phosphorylated p217+ tau protein in a sample are provided. Methods of detecting or diagnosing tauopathies, methods of determining the effectiveness of a treatment of a tauopathy, and methods of determining whether a subject is suitable for anti-p217+ tau antibody therapy are also provided. Also described are antibodies for use in the methods and kits comprising the antibodies.

### CROSS-REFERENCE TO RELATED APPLICATIONS

This application is a continuation of U.S. patent application Ser. No. 16/978,006, filed on Sep. 3, 2020, which is a National Stage of International Application No. PCT/IB2019/051747, filed on Mar. 4, 2019, which claims the benefit of priority to U.S. Provisional Application No. 62/638,524, filed on Mar. 5, 2018, each of which is hereby incorporated by reference in its entirety for all purposes.

### SEQUENCE LISTING

The present application contains a Sequence Listing which has been submitted electronically in XML file format and is hereby incorporated by reference in its entirety. Said XML copy, created on Jun. 18, 2025, is named JNJ_005_US4_SL.xml and is 62,055 bytes in size.

### FIELD OF THE INVENTION

The invention relates to compositions and methods for detecting neurodegeneration. In particular, the invention relates to methods of measuring the amount of singly- or multiply-phosphorylated p217+ tau protein species in a biological sample and uses thereof, as well as antibodies and kits for use in the methods.

### BACKGROUND OF THE INVENTION

Alzheimer's Disease (AD) is a degenerative brain disorder characterized clinically by progressive loss of memory, cognition, reasoning, judgment and emotional stability that gradually leads to profound mental deterioration and ultimately death. AD is a very common cause of progressive mental failure (dementia) in aged humans and is believed to represent the fourth most common medical cause of death in the United States. AD has been observed in ethnic groups worldwide and presents a major present and future public health problem.

The brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles of paired helical filaments, are generally found in several areas of the human brain important for memory and cognitive function in patients with AD.

Neurofibrillary tangles are primarily composed of aggregates of hyper-phosphorylated tau protein. The main physiological function of tau is microtubule polymerization and stabilization. The binding of tau to microtubules takes place by ionic interactions between positive charges in the microtubule binding region of tau and negative charges on the microtubule lattice (Butner and Kirschner, J Cell Biol. 115(3):717-30, 1991). Tau protein contains 85 possible phosphorylation sites, and phosphorylation at many of these sites interferes with the primary function of tau. Tau that is bound to the axonal microtubule lattice is in a hypo-phosphorylation state, while aggregated tau in AD is hyper-phosphorylated, providing unique epitopes that are distinct from the physiologically active pool of tau (Iqbal et al., Curr Alzheimer Res. 7(8): 656-664, 2010).

The progression of tauopathy in an AD brain follows distinct spreading patterns. A tauopathy transmission and spreading hypothesis has been described based on the Braak stages of tauopathy progression in the human brain and tauopathy spreading after tau aggregate injections in preclinical tau models (Frost et al., J Biol Chem. 284:12845-52, 2009; Clavaguera et al., Nat Cell Biol. 11:909-13, 2009). It is believed that tauopathy can spread in a prion-like fashion from one brain region to the next. This spreading process would involve an externalization of tau seeds that can be taken up by nearby neurons and induce further tauopathy.

Fragments of tau protein in the neurofibrillary tangles move to the cerebrospinal fluid (CSF) where they can be harvested by lumbar puncture and measured by sensitive assays. The presence of neurological disease can thus be detected using assays that recognize tau protein-derived fragments in CSF. Such tau assays require the ability to recognize tau species characteristic of a neurodegenerative condition. Multiply-phosphorylated tau is the leading example of AD-associated tau protein. Therefore, assays that detect multiply-phosphorylated tau protein in CSF may be most effective in detecting the presence of AD.

Phosphorylation is not the only posttranslational modification to consider in measuring tau. Recent studies have demonstrated that in CSF, tau protein exists primarily as fragments rather than as full-length protein (Meredith et al. PLoS One. 8(10):e76523, 2013). Further, the tau fragmentation pattern may be influenced by disease, as proteolysis is frequently aberrant in pathological conditions. Consequently, tau-based assays for neurodegeneration need to provide information not only on the phosphorylation status (e.g. phosphorylation site), but also on the nature of the tau fragments (e.g. length of tau fragment, polarity) that are being measured. However, translation of this idea is hampered by the low endogenous levels of phosphorylated tau, especially in samples from healthy subjects.

In summary, there remains a need for sensitive, precise and accurate methods for detecting multiply-phosphorylated tau in biological fluids. Such methods would be useful to effectively detect, diagnose, stage and track disease progression of neurodegenerative diseases, such as AD and other tauopathies. The methods would also be useful as pharmacodynamics markers for measuring levels of total, free, and therapeutic antibody-bound multiply-phosphorylated tau. The ability to detect and measure multiply-phosphorylated tau fragments is of further importance to the field, as the transmissible tau species may be one or more tau fragments.

### BRIEF SUMMARY OF THE INVENTION

The invention satisfies the need to detect forms of tau in CSF that are linked to neurodegenerative diseases. The invention enables detection of singly- or multiply-phosphorylated tau as well as detection of tau fragments.

High sensitivity Enzyme Linked Immunoassays (ELISAs) according to embodiment the invention were developed and qualified for measuring a p217+ tau comprising a phosphorylated tau epitope (“p217+ tau epitope” or “pT3 epitope”) comprising phosphorylated residues T212 and/or T217 having the sequence of (212) R(pT)PSLPTPPTR (SEQ ID NO: 25), (217) RTPSLP(pT)PPTR (SEQ ID NO: 26) or (212&217) R(pT)PSLP(pT)PPTR (SEQ ID NO: 27).

Assays according to embodiments of the invention are capable of measuring p217+ tau species in various fluid matrices including but not limited to CSF, interstitial fluid (ISF), brain homogenate, serum, plasma and denatured or enriched versions thereof. Assays according to embodiments of the invention use a first monoclonal antibody directed to a pT3 epitope of tau as a capture antibody, and a second monoclonal antibody directed to a second epitope of tau as a detection antibody. The assays are highly sensitive, precise, accurate, transferrable between labs, dilution linear, and applicable to many sample types. In addition to measurement of p217+ tau species in raw biological fluid, the assays can be used to measure samples with or without denaturing, or after immunoprecipitation, two complementary techniques to quantify the amount of free p217+ tau or p217+ tau bound to an endogenous or therapeutically-administered antibody. The assays can be used in tandem with reverse phase High Performance Liquid Chromatography (rpHPLC) to measure fractionated CSF, allowing for analysis of the fragment profile of p217+ tau.

In one general aspect, the invention relates to a method of measuring the amount of p217+ tau peptides in a sample. The method comprises: (i) contacting the sample with a capture antibody directed against a p217+ tau epitope to capture p217+ tau peptides in the sample, and (ii) contacting the captured p217+ tau peptides with a detection antibody directed against an epitope comprising amino acid residues 119 to 126, such as amino acid residues 116-127, of tau protein, or an epitope containing amino acid residues 7 to 20 of tau protein to thereby measure an amount of p217+ tau peptides or an amount of long p217+ tau peptides, respectively, wherein the numbering of the amino acid is with reference to the amino acid sequence set forth in SEQ ID NO: 1.

In one particular aspect, the invention relates to a method of determining a relative amount of long p217+ tau peptides or short p217 tau peptide fragments in a sample. The method comprises (i) contacting the sample with a capture antibody directed against a p217+ tau epitope to capture p217+ tau peptides in the sample, (ii) contacting the captured p217+ tau peptides with a first detection antibody directed against an epitope comprising amino acid residues 119 to 126 of tau protein to thereby measure an amount of p217+ tau peptides, (iii) contacting the captured p217+ tau peptides with a second detection antibody directed against an epitope comprising amino acid residues 7 to 20 of tau protein to thereby measure an amount of long p217+ tau peptides, and (iv) determining a relative amount of long p217+ tau peptides or short p217+ tau peptides based on the amount of p217+ tau peptides and the amount of long p217+ tau peptides, wherein the numbering of the amino acid is with reference to the amino acid sequence set forth in SEQ ID NO: 1.

In one embodiment of the invention, an amount of short p217+ tau peptides in a sample is calculated based on the amount of p217+ tau peptides and the amount of long p217+ tau peptides in the sample, e.g., by subtracting the amount of long p217+ tau peptides from the amount of p217+ tau peptides. In another embodiment, a ratio between the amount of short p217+ tau peptides to the amount of p217+ tau peptides, a ratio between the amount of long p217+ tau peptides to the amount of p217+ tau peptides, or a ratio between the amount of long p217+ tau peptides to the amount of short p217+ tau peptides is determined based on the amount of p217+ tau peptides and the amount of long p217+ tau peptides in the sample. According to embodiments of the invention, the amount of p217+ tau peptides and/or the amount of long p217+ tau peptides in a sample, as well as information based on the measure amounts, such as the calculated amount of the short p217+ tau peptides and one or more of the ratios described above, can be used for one or more diagnostic purposes.

Accordingly, in one particular aspect, the invention relates to a method of determining a ratio of p217+ tau peptides to total tau peptides in a sample. The method comprises (i) contacting the sample with a capture antibody directed against a p217+ tau epitope to capture p217+ tau peptides in the sample, and contacting the sample with a phosphorylation-independent capture antibody directed against an epitope between amino acids 150 and 250 of tau protein, preferably an epitope comprising amino acids 159-163 of tau protein, to capture total tau peptides in the sample, (ii) conducting at least one of (a) contacting the captured p217+ tau peptides with a first detection antibody directed against an epitope comprising amino acid residues 119 to 126 of tau protein to thereby measure an amount of p217+ tau peptides, and contacting the capture total tau peptides with the first detection antibody to thereby measure an amount of total tau peptides; and (b) contacting the captured p217+ tau peptides with a second detection antibody directed against an epitope comprising amino acid residues 7 to 20 of tau protein to thereby measure an amount of long p217+ tau peptides, and contacting the capture total tau peptides with the second detection antibody to thereby measure an amount of total long tau peptides, and (iii) determining a ratio of the amount of the p217+ tau peptides to the amount of the total tau peptides, or a ratio of the amount of the long p217+ tau peptides to the amount of the total long tau peptides, wherein the numbering of the amino acid is with reference to the amino acid sequence set forth in SEQ ID NO: 1. In one embodiment, an amount of short p217+ tau peptides is calculated by subtracting the amount of long p217+ tau peptides from the amount of p217+ tau peptides, an amount of total short tau peptides is calculated by subtracting the amount of total long tau peptides from the amount of total tau peptides, and a ratio of the amount of short p217+ tau peptides to the amount of the total short tau peptides is determined.

According to a particular aspect, a method of the invention comprises (i) contacting a biological sample, preferably a CSF sample, from a subject, with a capture antibody directed against a p217+ tau epitope to capture p217+ tau peptides in the sample, (ii) conducting at least one of (a) contacting the captured p217+ tau peptides with a first detection antibody directed against an epitope comprising amino acid residues 119 to 126 of tau protein to thereby measure an amount of p217+ tau peptides, and (b) contacting the captured p217+ tau peptides with a second detection antibody directed against an epitope comprising amino acid residues 7 to 20 of tau protein to thereby measure an amount of long p217+ tau peptides, and (iii) determining whether or not the subject suffers from a tauopathy or is at risk of developing a tauopathy based on at least one of the amount of p217+ tau peptides, the amount of long p217+ tau peptides, an amount of short p217+ tau peptides obtained by subtracting the amount of long p217+ tau peptides from the amount of p217+ tau peptides, and ratios thereof, wherein the numbering of the amino acid is with reference to the amino acid sequence set forth in SEQ ID NO: 1. In one embodiment, the method further comprises administering to the subject a therapeutic agent for treating or preventing the tauopathy.

According to a particular aspect, a method of the invention comprises (i) contacting a biological sample, preferably a CSF sample, from a subject, with a capture antibody directed against a p217+ tau epitope to capture p217+ tau peptides in the sample, and contacting the sample with a phosphorylation-independent capture antibody directed against an epitope between amino acids 150 and 250 of tau protein, preferably an epitope comprising amino acids 159-163 of tau protein, to capture total tau peptides in the sample, (ii) conducting at least one of (a) contacting the captured p217+ tau peptides with a first detection antibody directed against an epitope comprising amino acid residues 119 to 126 of tau protein to thereby measure an amount of p217+ tau peptides, and contacting the capture total tau peptides with the first detection antibody to thereby measure an amount of total tau peptides; and (b) contacting the captured p217+ tau peptides with a second detection antibody directed against an epitope comprising amino acid residues 7 to 20 of tau protein to thereby measure an amount of long p217+ tau peptides, and contacting the capture total tau peptides with the second detection antibody to thereby measure an amount of total long tau peptides, and (iii) determining whether or not the subject suffers from a tauopathy or is at risk of developing a tauopathy based on at least one of (a) a ratio of the amount of p217+ tau peptides to the amount of total tau peptides, (b) a ratio of the amount of long p217+ tau peptides to the amount of total long tau peptides, and (c) a ratio of an amount of short p217+ tau peptides to the amount of total short tau peptides, wherein the amount of short p217+ tau peptides is obtained by subtracting the amount of long p217+ tau peptides from the amount of p217+ tau peptides, and the amount of total short tau peptides is obtained by subtracting the amount of total short tau peptides from the amount of total tau peptides, wherein the numbering of the amino acid is with reference to the amino acid sequence set forth in SEQ ID NO: 1. In one embodiment, the method further comprises administering to the subject a therapeutic agent for treating or preventing the tauopathy.

According to another particular aspect, a method of the invention comprises (i) contacting a biological sample, preferably a CSF sample, from a subject under a treatment, with a capture antibody directed against a p217+ tau epitope to capture p217+ tau peptides in the sample, (ii) conducting at least one of (a) contacting the captured p217+ tau peptides with a first detection antibody directed against an epitope comprising amino acid residues 119 to 126 of tau protein to thereby measure an amount of p217+ tau peptides, and (b) contacting the captured p217+ tau peptides with a second detection antibody directed against an epitope comprising amino acid residues 7 to 20 of tau protein to thereby measure an amount of long p217+ tau peptides, and (iii) determining the effectiveness of the treatment in the subject based on at least one of the amount of p217+ tau peptides, the amount of long p217+ tau peptides, an amount of short p217+ tau peptides obtained by subtracting the amount of long p217+ tau peptides from the amount of p217+ tau peptides, and ratios thereof, wherein the numbering of the amino acid is with reference to the amino acid sequence set forth in SEQ ID NO: 1. In one embodiment, the method further comprises administering to the subject a therapeutic agent for treating or preventing the tauopathy.

According to another particular aspect, a method of the invention comprises (i) contacting a biological sample, preferably a CSF sample, from a subject under a treatment, with a capture antibody directed against a p217+ tau epitope to capture p217+ tau peptides in the sample, and contacting the sample with a phosphorylation-independent capture antibody directed against an epitope between amino acids 150 and 250 of tau protein, preferably an epitope comprising amino acids 159-163 of tau protein, to capture total tau peptides in the sample, (ii) conducting at least one of (a) contacting the captured p217+ tau peptides with a first detection antibody directed against an epitope comprising amino acid residues 119 to 126 of tau protein to thereby measure an amount of p217+ tau peptides, and contacting the capture total tau peptides with the first detection antibody to thereby measure an amount of total tau peptides; and (b) contacting the captured p217+ tau peptides with a second detection antibody directed against an epitope comprising amino acid residues 7 to 20 of tau protein to thereby measure an amount of long p217+ tau peptides, and contacting the capture total tau peptides with the second detection antibody to thereby measure an amount of total long tau peptides, and (iii) determining the effectiveness of the treatment in the subject based on at least one of (a) a ratio of the amount of p217+ tau peptides to the amount of total tau peptides, (b) a ratio of the amount of long p217+ tau peptides to the amount of total long tau peptides, and (c) a ratio of an amount of short p217+ tau peptides to the amount of total short tau peptides, wherein the amount of short p217+ tau peptides is obtained by subtracting the amount of long p217+ tau peptides from the amount of p217+ tau peptides, and the amount of total short tau peptides is obtained by subtracting the amount of total short tau peptides from the amount of total tau peptides, wherein the numbering of the amino acid is with reference to the amino acid sequence set forth in SEQ ID NO: 1. In one embodiment, the method further comprises administering to the subject a therapeutic agent for treating or preventing the tauopathy.

According to another particular aspect, a method of the invention comprises (i) contacting a biological sample, preferably a CSF sample, from a subject, with a capture antibody directed against a p217+ tau epitope to capture p217+ tau peptides in the sample, (ii) conducting at least one of (a) contacting the captured p217+ tau peptides with a first detection antibody directed against an epitope comprising amino acid residues 119 to 126 of tau protein to thereby measure an amount of p217+ tau peptides, and (b) contacting the captured p217+ tau peptides with a second detection antibody directed against an epitope comprising amino acid residues 7 to 20 of tau protein to thereby measure an amount of long p217+ tau peptides, and (iii) determining whether or not the subject is suitable for an anti-p217+ tau antibody based on at least one of the amount of p217+ tau peptides, the amount of long p217+ tau peptides, an amount of short p217+ tau peptides obtained by subtracting the amount of long p217+ tau peptides from the amount of p217+ tau peptides, and ratios thereof, wherein the numbering of the amino acid is with reference to the amino acid sequence set forth in SEQ ID NO: 1. In one embodiment, the method further comprises administering to the subject an anti-p217+ tau antibody for treating or preventing the tauopathy.

According to another particular aspect, a method of the invention comprises (i) contacting a biological sample, preferably a CSF sample, from a subject, with a capture antibody directed against a p217+ tau epitope to capture p217+ tau peptides in the sample, and contacting the sample with a phosphorylation-independent capture antibody directed against an epitope between amino acids 150 and 250 of tau protein, preferably an epitope comprising amino acids 159-163 of tau protein, to capture total tau peptides in the sample, (ii) conducting at least one of (a) contacting the captured p217+ tau peptides with a first detection antibody directed against an epitope comprising amino acid residues 119 to 126 of tau protein to thereby measure an amount of p217+ tau peptides, and contacting the capture total tau peptides with the first detection antibody to thereby measure an amount of total tau peptides; and (b) contacting the captured p217+ tau peptides with a second detection antibody directed against an epitope comprising amino acid residues 7 to 20 of tau protein to thereby measure an amount of long p217+ tau peptides, and contacting the capture total tau peptides with the second detection antibody to thereby measure an amount of total long tau peptides, and (iii) determining whether or not the subject is suitable for an anti-p217+ tau antibody therapy based on at least one of (a) a ratio of the amount of p217+ tau peptides to the amount of total tau peptides, (b) a ratio of the amount of long p217+ tau peptides to the amount of total long tau peptides, and (c) a ratio of an amount of short p217+ tau peptides to the amount of total short tau peptides, wherein the amount of short p217+ tau peptides is obtained by subtracting the amount of long p217+ tau peptides from the amount of p217+ tau peptides, and the amount of total short tau peptides is obtained by subtracting the amount of total short tau peptides from the amount of total tau peptides, wherein the numbering of the amino acid is with reference to the amino acid sequence set forth in SEQ ID NO: 1. In one embodiment, the method further comprises administering to the subject an anti-p217+ tau antibody for treating or preventing the tauopathy.

In another particular aspect, the invention relates to a method of monitoring a treatment with an anti-p217+ tau antibody in a subject, the method comprising: (i) obtaining a biological sample from the subject, (ii) separating the biological sample into a first sample containing p217+ tau free of the anti-p217+ tau antibody, preferably from of IgG, and a second sample containing p217+ tau peptides bound to the anti-p217+ tau antibody, (iii) obtaining a third sample containing p217+ tau free of anti-p217+ tau antibody from the second sample, preferably via rpHPLC, (iv) contacting each of the first sample and the third sample with a capture antibody directed against a p217+ tau epitope to capture p217+ tau peptides in each of the samples, (v) conducting at least one of (a) contacting the captured p217+ tau peptides with a first detection antibody directed against an epitope comprising amino acid residues 119 to 126 of tau protein to thereby measure an amount of p217+ tau peptides in each of the samples, and (b) contacting the captured p217+ tau peptides with a second detection antibody directed against an epitope comprising amino acid residues 7 to 20 of tau protein to thereby measure an amount of long p217+ tau peptides in each of the samples, (vi) monitoring the treatment with the anti-p217+ tau antibody based on at least one of the amount of the p217+ tau peptides and the amount of the long p217+ tau peptides in each of the samples, wherein the numbering of the amino acid is with reference to the amino acid sequence set forth in SEQ ID NO: 1. For example, the treatment with the anti-p217+ tau antibody can be monitored based on a ratio of the amount of long p217+ tau peptides in the first sample to that in the third sample, a ratio of the amount of the p217+ tau peptides in the first sample to that in the third sample, or a ratio of the amount of short p217+ tau peptides (which can be calculated by subtracting the amount of the long p217+ tau peptides from the amount of the p217+ tau peptides) in the first sample to that in the third sample. In one embodiment, the method further comprises administering to the subject an anti-p217+ tau antibody for treating or preventing the tauopathy.

In another general aspect, the invention relates to a method of monitoring a treatment with an anti-p217+ tau antibody in a subject, the method comprising: (i) obtaining a biological sample from the subject, (ii) obtaining a semi-denatured sample from the biological sample containing total p217+ tau, wherein the semi-denatured sample is heated to denature the antibodies in the sample, and obtaining a non-denatured sample from the biological sample containing p217+ tau free of the anti-p217+ tau antibody, (iii) contacting each of the semi-denatured sample and the non-denatured sample with a capture antibody directed against a p217+ tau epitope to capture p217+ tau peptides in each of the samples, (v) conducting at least one of (a) contacting the captured p217+ tau peptides with a first detection antibody directed against an epitope comprising amino acid residues 119 to 126 of tau protein to thereby measure an amount of p217+ tau peptides in each of the samples, and (b) contacting the captured p217+ tau peptides with a second detection antibody directed against an epitope comprising amino acid residues 7 to 20 of tau protein to thereby measure an amount of long p217+ tau peptides in each of the samples, and vi) monitoring the treatment with the anti-p217+ tau antibody based on at least one of the amount of the p217+ tau peptides and the amount of the long p217+ tau peptides in each of the samples, wherein the numbering of the amino acid is with reference to the amino acid sequence set forth in SEQ ID NO: 1. For example, the treatment with the anti-p217+ tau antibody can be monitored based on a ratio of the amount of the long p217+ tau peptides in the semi-denatured sample to that in the non-denatured sample, a ratio of the amount of the p217+ tau peptides in the semi-denatured sample to that in the non-denatured sample, or a ratio of the amount of the short p217+ tau peptides (which can be calculated by subtracting the amount of the long p217+ tau peptides from the amount of the p217+ tau peptides) in the semi-denatured sample to that in the non-denatured sample. In one embodiment, the method further comprises administering to the subject an anti-p217+ tau antibody for treating or preventing the tauopathy.

According to a particular aspect, the tauopathy includes, but is not limited to, one or more selected from the group consisting of Alzheimer's disease (including familial Alzheimer's disease and sporadic Alzheimer's disease), frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Preferably, the tauopathy is Alzheimer's disease (including familial Alzheimer's disease and sporadic Alzheimer's disease), FTDP-17 or progressive supranuclear palsy.

Most preferably, the tauopathy is Alzheimer's disease (including familial Alzheimer's disease and sporadic Alzheimer's disease).

According to a particular aspect, the lower limit of quantification of a method of the invention is about 40 fg/ml p217+ tau peptides and the lower limit of detection of a method of the invention is about 2 fg/ml p217+ tau peptides.

According to a particular aspect, the sample is a biological sample, such as a blood, brain homogenate, or cerebral spinal fluid (CSF) sample, from a subject in need thereof. Preferably, the biological sample is a CSF sample from a subject in need of a diagnosis of tauopathy, monitoring the effectiveness of a tauopathy treatment, or determination on the suitability for an anti-p217+ tau antibody therapy.

According to a particular aspect, a capture antibody useful for methods of the invention is directed against a p217+ tau epitope, preferably a p217+ tau epitope containing the amino acid sequence of SEQ ID NO: 25, 26 or 27. In one embodiment, a capture antibody useful for methods of the invention comprises immunoglobulin heavy chain HCDR1, HCDR2 and HCDR3 having the polypeptide sequences of SEQ ID NOs: 32, 33 and 34, respectively, and immunoglobulin light chain LCDR1, LCDR2 and LCDR3 having the polypeptide sequences of SEQ ID NOs: 35, 36 and 37, respectively. Preferably, the capture antibody has a heavy chain variable region comprising polypeptide sequence of SEQ ID NO: 28 or 30 and a light chain variable region having the polypeptide sequence of SEQ ID NO: 29 or 31.

According to a particular aspect, a detection antibody useful for methods of the invention is directed against an epitope comprising amino acid residues 119 to 126 of tau protein, preferably an epitope comprising the amino acid sequence of SEQ ID NO: 10, such as the amino acid sequence of SEQ ID NO:11. In one embodiment, a detection antibody useful for methods of the invention comprises immunoglobulin heavy chain HCDR1, HCDR2 and HCDR3 having the polypeptide sequences of SEQ ID NOs: 2, 3 and 4, respectively; and immunoglobulin light chain LCDR1, LCDR2 and LCDR3 having the polypeptide sequences of SEQ ID NOs: 5, 6 and 7, respectively. Preferably, the detection antibody is a pT82 antibody comprising a heavy chain variable region having the polypeptide sequence of SEQ ID NO: 8 and a light chain variable region having the polypeptide sequence of SEQ ID NO: 9.

According to another particular aspect, a detection antibody useful for methods of the invention is directed against an epitope containing amino acid residues 7 to 20 of tau protein, preferably an epitope having the amino acid sequence of SEQ ID NO: 20. In one embodiment, a detection antibody useful for methods of the invention comprises immunoglobulin heavy chain HCDR1, HCDR2 and HCDR3 having the polypeptide sequences of SEQ ID NOs: 12, 13 and 14, respectively; and immunoglobulin light chain LCDR1, LCDR2 and LCDR3 having the polypeptide sequences of SEQ ID NOs: 15, 16 and 17, respectively. Preferably, the detection antibody is a hT43 antibody comprising a heavy chain variable region having the polypeptide sequence of SEQ ID NO: 18 and a light chain variable region having the polypeptide sequence of SEQ ID NO: 19.

According to another particular aspect, a phosphorylation-independent capture antibody useful for the invention is directed against an epitope between amino acids 150 and 250 of tau protein, preferably an epitope comprising amino acids 211 to 221 of tau protein, or an epitope comprising amino acids 159 to 163 of tau protein, more preferably an epitope having the amino acid sequence of SEQ ID NO:21. In one embodiment, a phosphorylation-independent capture antibody useful for the invention is a hT7 antibody.

According to another particular aspect, the sample used in methods of the invention is obtained after fractionating a biological sample using reverse phase high-performance liquid chromatography (rpHPLC).

In another general aspect, the invention relates to an isolated detection antibody or antigen-binding fragment thereof that binds to a tau protein at an epitope comprising amino acid residues 7 to 20 of tau protein, comprising (a) immunoglobulin heavy chain HCDR1, HCDR2 and HCDR3 having the polypeptide sequences of SEQ ID NOs: 12, 13 and 14, respectively; and (b) immunoglobulin light chain LCDR1, LCDR2 and LCDR3 having the polypeptide sequences of SEQ ID NOs: 15, 16 and 17, respectively. According to a particular aspect, the isolated detection antibody or antigen-binding fragment thereof comprises a heavy chain variable region having a polypeptide sequence of SEQ ID NO: 18, and a light chain variable region having a polypeptide sequence of SEQ ID NO: 19. Preferably, the isolated detection antibody or antigen-binding fragment thereof that binds to a tau protein at an epitope comprising amino acid residues 7 to 20 of tau protein is a hT43 antibody.

In another general aspect, the invention relates to a kit comprising (a) a capture antibody directed against a p217+ tau epitope, and (b) a detection antibody directed against a tau protein epitope comprising amino acid residues 7 to 20 or 116 to 127 of tau protein. Optionally, the kit further comprises a phosphorylation-independent capture antibody directed against a tau epitope between amino acids 150 and 250 of tau protein. The kit can be used, for example, to measure the amount of p217+ tau peptides, the amount of long p217+ tau peptides, the amount of short p217+ tau peptides, the ratio of the amount of short p217+ tau peptides to the amount of long p217+ tau peptides, the ratio of the amount of short p217+ tau peptides to the amount of total short tau peptides in a sample, etc. The kit can also be for various diagnostic or monitoring purposes, e.g., to determine whether or not a subject suffers from a tauopathy or is at risk of developing a tauopathy, monitoring the efficacy of a treatment against a tauopathy, such as a treatment with an anti-p217+ tau antibody, to determine whether or not the subject is suitable for an anti-p217+ tau antibody, etc.

According to a particular aspect, a kit of the invention comprises a capture antibody, which has immunoglobulin heavy chain HCDR1, HCDR2 and HCDR3 having the polypeptide sequences of SEQ ID NOs: 32, 33 and 34, respectively, and immunoglobulin light chain LCDR1, LCDR2 and LCDR3 having the polypeptide sequences of SEQ ID NOs: 35, 36 and 37, respectively. Preferably, the capture antibody has a heavy chain variable region comprising polypeptide sequence of SEQ ID NO: 28 and a light chain variable region having the polypeptide sequence of SEQ ID NO: 29.

According to another particular aspect, a kit of the invention comprises a detection antibody, which comprises immunoglobulin heavy chain HCDR1, HCDR2 and HCDR3 having the polypeptide sequences of SEQ ID NOs: 2, 3 and 4, respectively; and immunoglobulin light chain LCDR1, LCDR2 and LCDR3 having the polypeptide sequences of SEQ ID NOs: 5, 6 and 7, respectively. Preferably, the detection antibody is a pT82 antibody comprising a heavy chain variable region having the polypeptide sequence of SEQ ID NO: 8 and a light chain variable region having the polypeptide sequence of SEQ ID NO: 9.

According to another particular aspect, a kit of the invention comprises a detection antibody, which comprises immunoglobulin heavy chain HCDR1, HCDR2 and HCDR3 having the polypeptide sequences of SEQ ID NOs: 12, 13 and 14, respectively; and immunoglobulin light chain LCDR1, LCDR2 and LCDR3 having the polypeptide sequences of SEQ ID NOs: 15, 16 and 17, respectively. Preferably, the detection antibody is a hT43 antibody comprising a heavy chain variable region having the polypeptide sequence of SEQ ID NO: 18 and a light chain variable region having the polypeptide sequence of SEQ ID NO: 19.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows a representative standard curve for the pT3xhT43 and pT3xpT82 assays generated using calibrant peptides with mean+/−SD of duplicate measurements shown at each point.

FIGS. 2A-2E show the dilution linearity of pT3xhT43 and pT3xpT82 assays in CSF samples with measurements shown (A, C and E) in dilution corrected pg/ml or (B and D) as dilution corrected % of 1:4 measurement, with dashed lines indicating +/−20% of 1:4 measurements.

FIG. 3 shows the intra- and inter-test precision of the (A) pT3xhT43 and (B) pT3xpT82 assays.

FIGS. 4A-4B show the precision between testing sites of the pT3xhT43 and pT3xpT82 assays with the data graphed as Signal/Noise (S/N).

FIGS. 5A-5B show the competition of pT3-based assay signal by soluble p217+ tau-targeted antibodies on (A) pT3xhT43 and (B) pT3xpT82 assays.

FIG. 6 shows the phosphorylation dependency of the pT3xhT43 and pT3xpT82 assays.

FIG. 7 shows a p217+ tau fragment profile of AD CSF, measured using the pT3xhT43 and pT3xpT82 assays, with data graphed as signal minus noise.

FIGS. 8A-8B show the temperature and freeze-thaw stability of p217+ tau signal in an AD CSF sample using the (A) pT3xhT43 and (B) hT7xpT82 assays.

FIG. 9 shows the long term stability of p217+ tau signal in CSF samples after storage at −70° C. No change in signal was detected.

FIGS. 10A-10F show a correlation between p217+ tau and the classical AD biomarkers Aβ42, tTau (total Tau), and pTau181, as measured by (A-C) pT3xhT43 and (D-F) pT3xpT82 assay.

FIGS. 11A-11B show a correlation between brain biopsy IHC analysis and p217+ tau, as measured by (A) pT3xhT43 and (B) pT3xpT82 assays.

FIGS. 12A-12D show the results of (A) pT3xhT43, (B) pT3xpT82, (C) hT7xpT82, and (D) ratio of pT3xpT82 vs. hT7xpT82 analysis of crude CSF from AD and HV patients.

FIG. 13 shows the predictive power of the pT3xhT43 (“343”), pT3xpT82 (“382”), and hT7xpT82 (“782”) assays in differentiating AD from HV subjects.

FIGS. 14A-14F show the signal from (A, B) pT3xhT43 (“343”), (C, D) pT3xpT82 (“382”), and (E, F) hT7xpT82 (“782”) assays carried out on rp-HPLC fractions of CSF from (A, C, E) AD and (B, D, F) HV subjects.

FIGS. 15A-15O show the signal from (A-E) pT3xhT43, (F-J) pT3xpT82, and (K-O) hT7xpT82 assays carried out on rp-HPLC fractions of CSF from CDR 0 and CDR 0.5 subjects.

FIGS. 16A-16B show the results of (A) ratio of pT3xpT82 vs hT7xpT82 analysis (pTau short) or ratio of pT3xhT43 vs hT7xpT82 analysis (pTau long) on crude CSF and (B) ratio of pT3xpT82 vs. hT7xpT82 analysis of rp-HPLC fractions of CSF, compared to MMSE score; all from a blinded cohort of CDR 0 and CDR1 subjects.

FIGS. 17A-17T show the results of (A) pT3xhT43, (B) pT3xpT82, and (C) hT7xpT82 analysis on crude CSF, (D) correlation of the two pT3 assays on crude CSF, (E) correlation of pT3xpT82 vs hT7xpT82 on crude CSF, (F) correlation of pT3xhT43 vs Innotest tTau on crude CSF, (G) correlation of pT3xhT43 vs Innotest pTau181 on crude CSF, (H) correlation of pT3xhT43 vs Innotest AB42 on crude CSF, (I) correlation of pT3xhT43 vs Innotest AB42/40 ratio on crude CSF; (J-I) pT3xhT43 signal, (M-P) pT3xpT82 signal, or (Q-T) hT7xpT82 signal in (J,M,Q) all rp-HPLC fractions as well as (K, N, R) sums of all fractions, (O,S) sums of early peak fractions (short tau fragments), or (L,P,T) sums of late peak fractions (larger tau fragments); all from a cohort of HV, MCI, and AD subjects.

FIGS. 18A-18P show the results of (A) pT3xpT82 (p217+ short) vs pT3xhT43 (p217+ long), (B) pT3xpT82 vs hT7xpT82 (tTau short), (C) pT3xpT82 vs. NFL, and (D) pT3xhT43 or (E) pT3xpT82 vs amyloid status, as well as (F-I, N-P) pT3xhT43 or (J-M) pT3xpT82 correlation with (F-M) various cognition scores or (N-P) change in these scores over 78 weeks; all from cohort of 235 subjects (90 of which had the 78 week follow up) from Janssen study ELN115727301/302 on mild-moderate AD subjects. The subjects were initially enrolled (and classified as AD) based on cognition, however upon biochemical evaluation (AB40 and AB42) it was determined that 27 of the subjects were amyloid negative, and thus likely represent dementia of non-AD causes. These subjects are analyzed as a separate cohort in the figures above and designated as amyloid negative=0, while the amyloid positive subjects=1.

FIG. 19 shows the signal from a pT3xhT43 assay carried out on rp-HPLC fractions of AD CSF samples spiked with IgG, pT3 mAb, humanized pT3 mAb or mock control, followed by immunoprecipitation to collect antibody-bound p217+ tau.

FIGS. 20A-20B show the antibody dose dependency of the immunocapture/rpHPLC method for quantifying (A) antibody-free and (B) antibody-bound p217+ tau, with data graphed as the sum of signal in rpHPLC fractions 12-16.

FIGS. 21A-21C show the differential kinetics of antibody vs. p217+ tau damage either (A, C) with or (B) without heat-mediated denaturation. (A) humanized PT3 mAb/CSF mix; (B) untreated CSF; (C) humanized PT3 mAb.

FIGS. 22A-22C show (A) heat-mediated denaturation and (B) immunocapture/rpHPLC methods for quantifying antibody-free vs. antibody-bound p217+ tau.; (C) shows a comparison of the methods.

FIG. 23 shows a lack of pT3-based assay recognition of p217+ tau in Cynomolgus Macaque CSF.

FIGS. 24A-24C show measurements of p217+ tau in Marmoset CSF as determined using the (A) pT3xhT43, (B) pT3xpT82 and (C) hT7xpT82 assays.

FIGS. 25A-25D show measurements of (A,B) hT7xpT82 (tTau) or (C,D) pT3xpT82 (p217+ tau short) in crude serum from 4 AD and 4HV subjects. Measurements were performed at (A,C) 1:4 or (B,D) 1:16 dilution, note lack of dilution linearity and sensitivity

FIGS. 26A-26B show measurements of (A) hT7xpT82 (tTau) or (B) pT3xpT82 (p217+ tau short) in serums pretreated with NaOAc and heat denaturation, from the same 4 AD and 4HV subjects evaluated in FIGS. 25A-25D.

FIG. 27 shows measurements of pT3xpT82 (p217+ tau short) in pT3-immunoprecipitations (IP) of serums from the same 4 AD and 4HV subjects evaluated in FIGS. 25A-25D and FIGS. 26A-26B.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

### Definitions

Unless defined otherwise, all technical and scientific terms used herein have the same meaning commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

As used herein, the term “antibody” or “immunoglobulin” is used in a broad sense and includes immunoglobulin or antibody molecules including polyclonal antibodies, monoclonal antibodies including murine, human, human-adapted, humanized and chimeric monoclonal antibodies and antibody fragments.

In general, antibodies are proteins or peptide chains that exhibit binding specificity to a specific antigen. Antibody structures are well known. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of any vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from mouse antibodies or human antibodies.

In addition to the heavy and light constant domains, antibodies contain light and heavy chain variable regions. An immunoglobulin light or heavy chain variable region consists of a “framework” region interrupted by “antigen-binding sites.” The antigen-binding sites are defined using various terms and numbering schemes as follows:
(i) Kabat: “Complementarity Determining Regions” or “CDRs” are based on sequence variability (Wu and Kabat, J Exp Med. 132:211-50, 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL));
(ii) Chothia: The term “hypervariable region,” “HVR” refers to the regions of an antibody variable domain which are hypervariable in structure as defined by Chothia and Lesk (Chothia and Lesk, J Mol Biol. 196:901-17, 1987). Generally, the antigen-binding site has three hypervariable regions in each VH (H1, H2, H3) and VL (L1, L2, L3). Numbering systems as well as annotation of CDRs and HVRs have been revised by Abhinandan and Martin (Abhinandan and Martin, Mol Immunol. 45:3832-9, 2008);
(iii) IMGT: Another definition of the regions that form the antigen-binding site has been proposed by Lefranc (Lefranc et al., Dev Comp Immunol. 27:55-77, 2003) based on the comparison of V domains from immunoglobulins and T-cell receptors. The International ImMunoGeneTics (IMGT) database (http:_//www_imgt_org) provides a standardized numbering and definition of these regions. The correspondence between CDRs, HVRs and IMGT delineations is described in Lefranc et al., 2003, Id.;
(iv) The antigen-binding site can also be delineated based on “Specificity Determining Residue Usage” (SDRU) (Almagro, Mol Recognit. 17:132-43, 2004), where SDR, refers to amino acid residues of an immunoglobulin that are directly involved in antigen contact.

“Framework” or “framework sequence” is the remaining sequences within the variable region of an antibody other than those defined to be antigen-binding site sequences. Because the exact definition of an antigen-binding site can be determined by various delineations as described above, the exact framework sequence depends on the definition of the antigen-binding site. The framework regions (FRs) are the more highly conserved portions of variable domains. The variable domains of native heavy and light chains each comprise four FRs (FR1, FR2, FR3 and FR4, respectively) which generally adopt a beta-sheet configuration, connected by the three hypervariable loops. The hypervariable loops in each chain are held together in close proximity by the FRs and, with the hypervariable loops from the other chain, contribute to the formation of the antigen-binding site of antibodies. Structural analysis of antibodies revealed the relationship between the sequence and the shape of the binding site formed by the complementarity determining regions (Chothia et al., J. Mol. Biol. 227: 799-817, 1992; Tramontano et al., J. Mol. Biol. 215:175-182, 1990). Despite their high sequence variability, five of the six loops adopt just a small repertoire of main-chain conformations, called “canonical structures.” These conformations are first of all determined by the length of the loops and secondly by the presence of key residues at certain positions in the loops and in the framework regions that determine the conformation through their packing, hydrogen bonding or the ability to assume unusual main-chain conformations.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the constant region of the heavy chain. According to other particular embodiments, the antigen-binding fragment comprises Fab and F(ab′).

As used herein, the term “epitope” refers to a site on an antigen to which an immunoglobulin, antibody, or antigen-binding fragment thereof, specifically binds. Epitopes can be formed both from contiguous amino acids or from noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

As used herein, the term “tau” or “tau protein” refers to an abundant central and peripheral nervous system protein having multiple isoforms. In the human central nervous system (CNS), six major tau isoforms ranging in size from 352 to 441 amino acids in length exist due to alternative splicing (Hanger et al., Trends Mol Med. 15:112-9, 2009). The isoforms differ from each other by the regulated inclusion of 0-2 N-terminal inserts, and 3 or 4 tandemly arranged microtubule-binding repeats, and are referred to as 0N3R, 1N3R, 2N3R, 0N4R, 1N4R and 2N4R. As used herein, the term “control tau” refers to the tau isoform of SEQ ID NO: 1 that is devoid of phosphorylation and other post-translational modifications. As used herein, the term “tau” includes proteins comprising mutations, e.g., point mutations, fragments, insertions, deletions and splice variants of full length wild type tau. The term “tau” also encompasses post-translational modifications of the tau amino acid sequence. Post-translational modifications include, but are not limited to, phosphorylation.

Unless otherwise indicated, as used herein, the numbering of the amino acid in a tau protein or fragment thereof is with reference to the amino acid sequence set forth in SEQ ID NO: 1.

As used herein, the term “p217+ tau peptides,” “p217+ tau,” or “p217+ tau protein” means a human tau protein or tau fragment that is phosphorylated at one or both of residue 217 (pT217) and residue 212 (pT212) of tau protein, wherein the numbering of the positions is according to the numbering in SEQ ID NO: 1.

As used herein, the term “p217+ tau epitope” refers to a tau epitope containing at least one of phosphorylated T217 and phosphorylated T212, wherein the numbering of the positions is according to the numbering in SEQ ID NO: 1. Examples of p217+ tau epitope include, e.g., a pT3 epitope. As used herein, the term “pT3 epitope” refers to an epitope containing amino acids 210-220 of human tau protein that is phosphorylated at least one reside of T217 and T212 of human tau, wherein the numbering of the positions is according to the numbering in SEQ ID NO: 1. Examples of pT3 epitope include, e.g., SEQ ID NO: 25, 26 and 27.

As used herein, each of the terms “long p217+ tau peptides,” “long p217+ tau,” “long form of p217+ tau peptides,” or “long p217+ tau peptides fragment” has the same meaning, referring to a p217+ tau peptides that comprises the p217+ tau epitope and an epitope comprising amino acid residues 7 to 20 of tau protein. The “long p217+ tau peptides” according to embodiments of the invention can have different lengths. For example, the amino-terminus of a “long p217+ tau peptides fragment” can be the amino acid residue 1, 2, 4, 5, 6, or 7 of tau protein.

As used herein, each of the terms “short p217+ tau peptides,” “short p217+ tau,” “short form of p217+ tau peptides,” or “short p217+ tau peptides fragment” has the same meaning, referring to a p217+ tau peptides that comprises the p217+ tau epitope and an epitope comprising amino acid residues 119 to 126 of tau protein, but does not contain an epitope comprising amino acid residues 7 to 20 of tau protein. The “short p217+ tau peptides” according to embodiments of the invention can have different lengths. For example, the amino-terminus of a “short p217+ tau peptides” can be any of the amino acid residues between the epitope comprising amino acid residues 7 to 20 of tau protein and the epitope comprising amino acid residues 119 to 126 of tau protein.

As used herein, each of the terms “long tau peptide,” “long tau,” “long form of tau peptide,” or “long tau peptide fragment” has the same meaning, referring to a tau peptide that comprises the tau epitope recognized by a phosphorylation-independent capture antibody and an epitope comprising amino acid residues 7 to 20 of tau protein. The “long tau peptide fragments” according to embodiments of the invention can have different lengths. For example, the amino-terminus of a “long tau peptide fragment” can be the amino acid residue 1, 2, 4, 5, 6, or 7 of tau protein.

As used herein, each of the terms “short tau peptide,” “short tau,” “short form of tau peptide,” or “short tau peptide fragment” has the same meaning, referring to a tau peptide that comprises the tau epitope recognized by a phosphorylation-independent capture antibody and an epitope comprising amino acid residues 119 to 126 of tau protein, but does not contain an epitope comprising amino acid residues 7 to 20 of tau protein. The “short tau peptide fragments” according to embodiments of the invention can have different lengths. For example, the amino-terminus of a “short tau peptide” can be any of the amino acid residues between the epitope comprising amino acid residues 7 to 20 of tau protein and the epitope comprising amino acid residues 119 to 126 of tau protein.

As used herein, the term “capture antibody” refers to an antibody that binds to an antigen of interest and is directly or indirectly linked to a solid support. Examples of solid supports include, but are not limited to, microparticles or beads, such as a magnetic beads. Examples of capture antibodies include, but are not limited to, a monoclonal antibody that binds to a p217+ tau epitope. According to embodiments of the invention, the capture antibody can be a monoclonal antibody comprising immunoglobulin heavy chain HCDR1, HCDR2 and HCDR3 having the polypeptide sequences of SEQ ID NOs: 32, 33 and 34, respectively, and immunoglobulin light chain LCDR1, LCDR2 and LCDR3 having the polypeptide sequences of SEQ ID NOs: 35, 36 and 37. In a particular embodiment, the capture antibody is pT3. As used herein, the term “pT3” refers to an antibody that binds to p217+ tau peptides and has a heavy chain variable region amino acid sequence of SEQ ID NO: 28 and a light chain variable region amino acid sequence of SEQ ID NO: 29. In one embodiment, the pT3 monoclonal antibody is expressed by a mouse-hybridoma. In another embodiment, the capture antibody is a humanized antibody having a heavy chain variable region amino acid sequence of SEQ ID NO: 30 and a light chain variable region amino acid sequence of SEQ ID NO: 31.

According to other embodiments of the invention, the capture antibody can be a monoclonal antibody that binds to an epitope between amino acids 150 and 250 of tau protein, preferably amino acids 211-221 or amino acids 159-163 of human tau protein, in a phosphorylation-independent manner, and the numbering of the positions is according to the numbering in SEQ ID NO: 1. In a particular embodiment, the capture antibody is hT7. As used herein, the term “hT7” refers to a publicly available monoclonal antibody that binds to an epitope comprising amino acids 159-163 of human tau protein, wherein the numbering of the positions is according to the numbering in SEQ ID NO: 1. A hT7 monoclonal antibody is commercially available, e.g., from ThermoFisher (e.g., Catalog #: MN1000).

As used herein, the term “detection antibody” refers to an antibody that binds to an antigen of interest and has a detectible label or is linked to a secondary detection system. Examples of detectable labels include, but are not limited to, various enzymes, prosthetic groups, fluorescent materials, luminescent materials, bioluminescent materials, and radioactive materials. Examples of detection antibodies include, but are not limited to, a monoclonal antibody that binds to tau protein, preferably an epitope comprising amino acids 7-20 or 116-127 of human tau protein, wherein the numbering of the positions is according to the numbering in SEQ ID NO: 1. When a monoclonal antibody that binds to a tau protein at an epitope comprising amino acids 7-20 is used as a detection antibody for captured p217+ tau peptides, long tau fragments are detected. When a monoclonal antibody that binds to a tau protein at an epitope comprising amino acids 116-127 is used as a detection antibody for captured p217+ tau peptides, both short and long tau fragments are detected.

In a particular embodiment, the detection antibody is hT43. As used herein, the term “hT43” refers to a monoclonal antibody that binds to an epitope comprising amino acids 7-20 of human tau protein, wherein the numbering of the positions is according to the numbering in SEQ ID NO: 1, and the antibody has a heavy chain variable region amino acid sequence of SEQ ID NO: 8 and a light chain variable region amino acid sequence of SEQ ID NO: 9. In another particular embodiment, the detection antibody is pT82. As used herein, the term “pT82” refers to a monoclonal antibody that binds to an epitope comprising amino acids 119-126, preferably 116-127, of human tau protein, wherein the numbering of the positions is according to the numbering in SEQ ID NO: 1, and the antibody has a heavy chain variable region amino acid sequence of SEQ ID NO: 18 and a light chain variable region amino acid sequence of SEQ ID NO: 19.

As used herein, the term “pT3-based assay” refers to an assay according to an embodiment of the invention wherein the pT3 antibody is used as the capture antibody. As used herein, the term “pT3xhT43” refers to an assay according to an embodiment of the invention wherein the pT3 antibody is used as the capture antibody and the hT43 antibody is used as the detection antibody. As used herein, the term “pT3xpT82” refers to an assay according to an embodiment of the invention wherein the pT3 antibody is used as the capture antibody and the pT82 antibody is used as the detection antibody.

As used herein, the term “hT7-based assay” refers to assays according to embodiments of the invention wherein the hT7 antibody is used as the capture antibody. As used herein, the term “hT7xpT82” refers to assays according to embodiments of the invention wherein the hT7 antibody is used as the capture antibody and the pT82 antibody is used as the detection antibody.

As used herein, the term “subject” refers to an animal, and preferably a mammal. According to particular embodiments, the subject is a mammal including a non-primate (e.g., a camel, donkey, zebra, cow, pig, horse, goat, sheep, cat, dog, rat, rabbit, guinea pig, marmoset or mouse) or a primate (e.g., a monkey, chimpanzee, or human). In particular embodiments, the subject is a human.

As used herein a “tauopathy” encompasses any neurodegenerative disease that involves the pathological aggregation of tau within the brain. In addition to familial and sporadic AD, other exemplary tauopathies are frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, and chronic traumatic encephalopathy, such as dementia pugulistica (boxing disease) (Morris et al., Neuron, 70:410-26, 2011).

As used herein, the terms “determining,” “measuring,” “assessing,” and “assaying” are used interchangeably and include both quantitative and qualitative determinations. These terms refer to any form of measurement, and include determining if a characteristic, trait, or feature is present or not. Assessing may be relative or absolute. “Assessing the presence of” includes determining the amount of something present, as well as determining whether it is present or absent.

As used herein, the term “diagnosis” means detecting a disease or disorder or determining the stage or degree of a disease or disorder, such as a tauopathy. Usually, a diagnosis of a disease or disorder is based on the evaluation of one or more factors and/or symptoms that are indicative of the disease. A diagnosis can be made based on the presence, absence or amount of a factor which is indicative of presence or absence of the disease or condition, e.g. p217+ tau. Each factor or symptom that is considered to be indicative for the diagnosis of a particular disease does not need be exclusively related to the particular disease, i.e. there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, e.g. an anti-p217+ tau antibody therapy, or predicting the pattern of response to a drug therapy, e.g. an anti-p217+ tau antibody therapy. The diagnostic methods may be used independently, or in combination with other diagnosing and/or staging methods known in the medical arts for a particular disease or disorder, e.g., Alzheimer's disease.

As used herein, the terms “increase” and “decrease” refer to differences in the quantity of a particular biomarker in a sample as compared to a control or reference level. For example, the quantity of particular peptide, may be present at an elevated amount or at a decreased amount in samples of patients with a disease compared to a reference level. In one embodiment, an “increase of a level” or “decrease of a level” may be a difference between the level of biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, an “increase of a level” or “decrease of a level” may be a statistically significant difference between the level of the biomarker present in a sample as compared to a control. For example, a difference may be statistically significant if the measured level of the biomarker falls outside of about 1.0 standard deviation, about 1.5 standard deviations, about 2.0 standard deviations, or about 2.5 stand deviations of the mean of any control or reference group. The reference or control can be, for example, a sample from a healthy individual, or a sample taken from the same individual at an earlier time point, such as a time point prior to administration of a therapeutic or an earlier time point during a therapeutic regimen.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids.

An “isolated antibody that binds to a tau protein” or an “isolated anti-tau antibody”, as used herein, is intended to refer to an antibody that specifically binds tau protein and which is substantially free of other antibodies having different antigenic specificities (for instance, an isolated anti-tau detection antibody is substantially free of antibodies that specifically bind antigens other than tau). An isolated anti-tau detection antibody can, however, have cross-reactivity to other related antigens, for instance from other species (such as tau species homologs).

As used herein, the term “specifically binds” or “specific binding” refers to the ability of an anti-tau antibody of the invention to bind to a predetermined target with a dissociation constant (K_{D}) of about 1×10⁻⁶ M or tighter, for example, about 1×10⁻⁷ M or less, about 1×10⁻⁸ M or less, about 1×10⁻⁹ M or less, about 1×10⁻¹⁰ M or less, about 1×10⁻¹¹ M or less, about 1×10⁻¹² M or less, or about 1×10⁻¹³ M or less. The KD is obtained from the ratio of Kd to Ka (i.e., Kd/Ka) and is expressed as a molar concentration (M). KD values for antibodies can be determined using methods in the art in view of the present disclosure. For example, the KD value of an anti-tau antibody can be determined by using surface plasmon resonance, such as by using a biosensor system, e.g., a Biacore® system, a Proteon instrument (BioRad), a KinExA instrument (Sapidyne), ELISA or competitive binding assays known to those skilled in the art. Typically, an anti-tau antibody binds to a predetermined target (i.e. tau) with a K_{D} that is at least ten fold less than its K_{D} for a nonspecific target as measured by surface plasmon resonance using, for example, a Proteon Instrument (BioRad). The anti-tau antibodies that specifically bind to tau can, however, have cross-reactivity to other related targets, for example, to the same predetermined target from other species (homologs), such as from mouse, rat, marmoset, dog or pig.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed detection antibody or antigen-binding fragment thereof that binds tau can be within the cytoplasm of a host cell, into the extracellular milieu such as the growth medium of a cell culture, or anchored to the cell membrane.

### Anti-Tau Antibodies

In one general aspect, the invention relates to isolated detection antibodies or antigen-binding fragments thereof that bind tau protein that has been immobilized by a capture antibody. Such anti-tau antibodies can have the properties of binding a phosphorylated epitope on tau or binding to a non-phosphorylated epitope on tau. Anti-tau detection antibodies can be useful as research or diagnostic reagents to detect tau in biological samples.

According to a particular aspect, the invention relates to an isolated detection antibody or antigen-binding fragment thereof that binds to a tau protein at an epitope comprising amino acid residues 119-126, preferably amino acid residues 116 to 127, of tau protein.

According to a particular aspect, the isolated detection antibody or antigen-binding fragment thereof that binds to tau protein at an epitope comprising amino acid residues 116 to 127 of tau protein comprises (a) immunoglobulin heavy chain HCDR1, HCDR2 and HCDR3 having the polypeptide sequences of SEQ ID NOs: 2, 3 and 4, respectively; and (b) immunoglobulin light chain LCDR1, LCDR2 and LCDR3 having the polypeptide sequences of SEQ ID NOs: 5, 6 and 7, respectively.

According to a particular aspect, the isolated detection antibody or antigen-binding fragment thereof that binds to tau protein at an epitope comprising amino acid residues 116 to 127 of tau protein comprises a heavy chain variable region having a polypeptide sequence at least 80%, preferably at least 85% or 90%, more preferably at least 95%, and most preferably 100%, identical to SEQ ID NO: 8 and a light chain variable region having a polypeptide sequence at least 80%, preferably at least 85% or 90%, more preferably at least 95%, and most preferably 100%, identical to SEQ ID NO: 9.

Preferably, the isolated detection antibody or antigen-binding fragment thereof that binds to tau protein at an epitope comprising amino acid residues 116 to 127 of tau protein is a pT82 antibody.

According to a particular aspect, the invention relates to an isolated detection antibody or antigen-binding fragment thereof that binds to a tau protein at an epitope comprising amino acid residues 7 to 20 of tau protein.

According to a particular aspect, the isolated detection antibody or antigen-binding fragment thereof that binds to tau protein at an epitope comprising amino acid residues 7 to 20 of tau protein comprises (a) immunoglobulin heavy chain HCDR1, HCDR2 and HCDR3 having the polypeptide sequences of SEQ ID NOs: 12, 13 and 14, respectively; and (b) immunoglobulin light chain LCDR1, LCDR2 and LCDR3 having the polypeptide sequences of SEQ ID NOs: 15, 16 and 17, respectively.

According to a particular aspect, the isolated detection antibody or antigen-binding fragment thereof that binds to tau protein at an epitope comprising amino acid residues 7 to 20 of tau protein comprises a heavy chain variable region having a polypeptide sequence at least 80%, preferably at least 85% or 90%, more preferably at least 95%, and most preferably 100%, identical to SEQ ID NO: 18 and a light chain variable region having a polypeptide sequence at least 80%, preferably at least 85% or 90%, more preferably at least 95%, and most preferably 100%, identical to SEQ ID NO: 19.

Preferably, the isolated detection antibody or antigen-binding fragment thereof that binds to tau protein at an epitope comprising amino acid residues 7 to 20 of tau protein is a hT43 antibody.

Antibodies of the present invention can be produced by a variety of techniques, for example by the hybridoma method (Kohler and Milstein, Nature. 256:495-7, 1975). Chimeric mAbs containing a light chain and heavy chain variable region derived from a donor antibody (typically murine) in association with light and heavy chain constant regions derived from an acceptor antibody (typically another mammalian species such as human) can be prepared by a method disclosed in U.S. Pat. No. 4,816,567. CDR-grafted mAbs having CDRs derived from a non-human donor immunoglobulin (typically murine) and the remaining immunoglobulin-derived parts of the molecule being derived from one or more human immunoglobulins can be prepared by techniques known to those skilled in the art such as that disclosed in U.S. Pat. No. 5,225,539. Fully human mAbs lacking any non-human sequences can be prepared from human immunoglobulin transgenic mice by techniques referenced in (Lonberg et al., Nature. 368:856-9, 1994; Fishwild et al., Nat Biotechnol. 14:845-51, 1996; Mendez et al., Nat Genet. 15:146-56, 1997). Human mAbs can also be prepared and optimized from phage display libraries (Knappik et al., J Mol Biol. 296:57-86, 2000; Krebs et al., J Immunol Methods. 254:67-84, 2001; Shi et al., J Mol Biol. 397:385-96, 2010).

The functional activity of detection antibodies and antigen-binding fragments thereof that bind tau can be characterized by methods known in the art. Methods for characterizing antibodies and antigen-binding fragments thereof that bind tau include, but are not limited to, affinity and specificity assays including Biacore, ELISA, and FACS analysis, immunohistochemistry analysis, etc.

Several well known methodologies can be employed to determine the binding epitope of the antibodies of the invention. For example, when the structures of both individual components are known, in silico protein-protein docking can be carried out to identify compatible sites of interaction. Hydrogen-deuterium (H/D) exchange can be carried out with the antigen and antibody complex to map regions on the antigen that are bound by the antibody. Segment and point mutagenesis of the antigen can be used to locate amino acids important for antibody binding. Co-crystal structure of antibody-antigen complex is used to identify residues contributing to the epitope and paratope.

In another general aspect, the invention relates to an isolated polynucleotide encoding a detection antibody or antigen-binding fragment thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding detection antibodies or antigen-binding fragments thereof of the invention can be altered without changing the amino acid sequences of the proteins. Exemplary isolated polynucleotides are polynucleotides encoding polypeptides comprising immunoglobulin heavy chain CDRs HCDR1, HCDR2 and HCDR3 shown in SEQ ID NOs: 2, 3 and 4, respectively, or polypeptides comprising immunoglobulin light chain CDRs LCDR1, LCDR2 and LCDR3 shown in SEQ ID NOs: 5, 6 and 7, respectively. Other exemplary isolated polynucleotides are polynucleotides encoding polypeptides comprising immunoglobulin heavy chain CDRs HCDR1, HCDR2 and HCDR3 shown in SEQ ID NOs: 12, 13 and 14, respectively, or polypeptides comprising immunoglobulin light chain CDRs LCDR1, LCDR2 and LCDR3 shown in SEQ ID NOs: 15, 16 and 17, respectively. Other exemplary isolated polynucleotides are polynucleotides encoding antibody variable regions of the invention. Other polynucleotides which, given the degeneracy of the genetic code or codon preferences in a given expression system, encode the antibodies of the invention are also within the scope of the invention. The isolated nucleic acids of the present invention can be made using well known recombinant or synthetic techniques. DNA encoding the monoclonal antibodies is readily isolated and sequenced using methods known in the art. Where a hybridoma is produced, such cells can serve as a source of such DNA. Alternatively, display techniques wherein the coding sequence and the translation product are linked, such as phage or ribosomal display libraries, can be used.

In another general aspect, the invention relates to a vector comprising an isolated polynucleotide encoding a detection antibody or antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention.

In another general aspect, the invention relates to a host cell comprising an isolated polynucleotide encoding a detection antibody or antigen-binding fragment thereof of the invention. Any host cell known to those skilled in the art in view of the present disclosure can be used for recombinant expression of antibodies or antigen-binding fragments thereof of the invention. Such host cells can be eukaryotic cells, bacterial cells, plant cells or archaeal cells. Exemplary eukaryotic cells can be of mammalian, insect, avian or other animal origins. Mammalian eukaryotic cells include immortalized cell lines such as hybridomas or myeloma cell lines such as SP2/0 (American Type Culture Collection (ATCC), Manassas, Va., CRL-1581), NS0 (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHO-K1 SV (Lonza Biologics), CHO-K1 (ATCC CRL-61, Invitrogen) or DG44.

In another general aspect, the invention relates to a method of producing a detection antibody or antigen-binding fragment thereof of the invention, comprising culturing a cell comprising a polynucleotide encoding the detection antibody or antigen-binding fragment thereof under conditions to produce a detection antibody or antigen-binding fragment thereof of the invention, and recovering the antibody or antigen-binding fragment thereof from the cell or cell culture (e.g., from the supernatant). Expressed antibodies or antigen-binding fragments thereof can be harvested from the cells and purified according to conventional techniques known in the art.

### Diagnostic Methods

The invention relates to measurement of p217+ tau species that are enriched in AD, e.g., by using a capture antibody, such as a pT3, which selectively immobilizes the p217+ tau species, in combination with an anti-tau detection antibody, which is labeled with a reporter element that allows detection of the captured p217+ tau species. Methods of the invention can be used for various diagnostic purposes, e.g., for diagnosing AD or other tauopathies in a subject, monitoring the effectiveness of a treatment, identifying a subject suitable for an anti-p217+ tau treatment, etc.

According to an embodiment of the invention, p217+ tau peptides in a sample of interest are captured with a capture antibody directed against a p217+ tau epitope, such as an epitope having the amino acid sequence of SEQ ID NO: 25, 26 or 27. The captured p217+ tau peptides, while all contain the p217+ tau epitope, may have different length, which can be detected by detection antibodies binding to different epitopes. For example, a detection antibody directed against an epitope comprising amino acid residues 7 to 20 of tau protein can only detect captured p217+ tau peptides or fragments thereof that still contain amino acid residues 7 to 20 of tau protein (“long p217+ tau peptides”), while a detection antibody directed against an epitope comprising amino acid residues 119 to 126 of tau protein can detect not only the long p217+ tau peptides, but also the short p217+ tau peptides. The captured p217+ tau peptides can be contacted with a detection antibody directed against an epitope comprising amino acid residues 7 to 20 or 116 to 127 of tau protein to thereby detect and measure the amount of the long p217+ tau peptides or the p217+ tau peptides (long and short p217+ tau peptides) in the sample. An amount of short p217+ tau peptides in a sample is calculated by subtracting the amount of long p217+ tau peptides from the amount of p217+ tau peptides.

According to another embodiment of the invention, in addition to capturing and measuring the amount of p217+ tau peptides in a sample, total tau peptides in the sample are captured with a phosphorylation-independent capture antibody, such as an antibody directed against an epitope between amino acids 150 and 250 of tau protein, preferably an epitope comprising amino acids 159-163 of tau protein. The captured total tau peptides can be contacted with a detection antibody directed against an epitope comprising amino acid residues 7 to 20 or 116 to 127 of tau protein to thereby detect and measure the amount of the total long tau peptides or the total tau peptides (long and short tau peptide fragments) in the sample. An amount of short total tau peptides in a sample is calculated by subtracting the amount of long total tau peptides from the amount of total tau peptides.

According to embodiments of the invention, a value related to p217+ tau peptides in a sample, such as the amount of p217+ tau peptides and the amount of long p217+ tau peptides, optionally the amount of total tau peptides and the amount of total long tau fragments, in a sample, as well as information based on the measure amounts, such as the calculated short p217+ tau peptides and short total tau peptides, or a ratio related to p217+ tau peptides, such as a ratio of the amount of short tau peptide fragments to the amount of long tau peptide fragments, a ratio of the amount of short p217+ tau peptides to the total amount of short tau fragments, a ratio of amount of long p217+ tau peptides to the total amount of long tau fragments, etc., can be used for one or more diagnostic purposes.

Diagnosis is performed by comparing a value related to p217+ tau peptides in a sample from a subject to corresponding baseline values. The baseline values can represent the mean levels in a population of healthy individuals. Baseline values can also represent previous levels determined in the same subject. In one embodiment, it is determined that a subject is suffering from a tauopathy if a value related to p217+ tau peptides in the biological sample from the subject, such as the amount of the long or short p217 tau peptides, or a ratio related to p217+ tau peptides, e.g., a ratio of the amount of short p217+ tau peptides to the amount of long p217+ tau peptides, is significantly higher than a corresponding baseline value. As used herein, “significant higher” refers to a higher value that is statistically significant, not due to chance alone, which has a p-value of 0.05 or less. A “significant higher” can be at least about 1%, 2%, 5%, or 10% higher than that found in healthy volunteers, at a p-value of less than 0.05, 0.04, 0.03, 0.01, 0.005, 0.001, etc.

In one embodiment, a method of the invention comprises (i) contacting a biological sample, preferably a CSF sample, with a capture antibody directed against an epitope comprising phosphorylated p217+ tau to capture p217+ tau peptides in the sample, (ii) contacting the captured p217+ tau peptides with a detection antibody directed against an epitope comprising amino acid residues 7 to 20 to thereby measure the amount of long p217+ tau peptides, and/or with a detection antibody directed against an epitope comprising amino acid residues 119 to 126 of tau protein to thereby measure the amount of long and short p217+ tau peptides in the sample, and (iii) determining whether or not the subject suffers from a tauopathy or is at risk of developing a tauopathy based on the amount of the p217+ tau peptides or the ratio of the amount of short p217+ tau peptides to the amount of long p217+ tau peptides. Diagnosis can be performed by comparing the amount or concentration of p217+ tau peptides in a sample from the subject to corresponding baseline values. Diagnosis can also be performed by comparing the ratio of the amount of short p217+ tau peptides to the amount of long p217+ tau peptides in a sample from the subject to corresponding baseline values.

In another embodiment, a method of the invention comprises (i) contacting a biological sample, preferably a CSF sample, with a capture antibody directed against a p217+ tau epitope to capture p217+ tau peptides in the sample, or with a phosphorylation-independent capture antibody directed against a tau epitope between amino acids 150 and 250 of tau protein to capture total tau peptides in the sample, (ii) contacting the captured p217+ tau peptides, or the captured total tau peptides, with a detection antibody directed against an epitope comprising amino acid residues 116 to 127 of tau protein to thereby measure the amount of long and short p217+ tau peptides, or the amount of total short tau peptides, in the sample, and (iii) determining whether or not the subject suffers from a tauopathy or is at risk of developing a tauopathy based on the amount of the ratio of the amount of short p217+ tau peptides to the amount of total short tau peptides in the biological sample. Diagnosis can be performed by comparing the ratio of the amount of short p217+ tau peptides to the amount of total short tau peptides comprising the same region of tau protein as that recognized by the pT3 antibody, i.e. amino acids 211-221 of tau, in a sample from the subject to corresponding baseline values.

In another embodiment, a method of the invention comprises (i) contacting a biological sample, preferably a CSF sample, with a capture antibody directed against a p217+ tau epitope to capture p217+ tau peptides in the sample, (ii) contacting the captured p217+ tau peptides with a detection antibody directed against an epitope comprising amino acid residues 7 to 20 to thereby measure the amount of long p217+ tau peptides, and/or with a detection antibody directed against an epitope comprising amino acid residues 116 to 127 of tau protein to thereby measure the amount of long and short p217+ tau peptides in the sample, and (iii) determining the effectiveness of the treatment in the subject based on the amount of the p217+ tau peptides or the ratio of the amount of short p217+ tau peptides to the amount of long p217+ tau peptides.

In yet another embodiment, a method of the invention comprises (i) contacting a biological sample, preferably a CSF sample, with a capture antibody directed against a p217+ tau epitope to capture p217+ tau peptides in the sample, or with a phosphorylation-independent capture antibody directed against a tau epitope between amino acids 150 and 250 of tau protein to capture total tau peptides in the sample, (ii) contacting the captured p217+ tau peptides, or the captured total tau peptides, with a detection antibody directed against an epitope comprising amino acid residues 116 to 127 of tau protein to thereby measure the amount of long and short p217+ tau peptides, or the amount of total short tau peptides, in the sample, and (iii) determining the effectiveness of the treatment in the subject based on the amount of the ratio of the amount of short p217+ tau peptides to the amount of total short tau peptides in the biological sample.

In yet another embodiment, the effectiveness of the treatment in the subject is determined by monitoring the amount of the p217+ tau peptides, the ratio of the amount of short p217+ tau peptides to the amount of long p217+ tau peptides, or the ratio of the amount of short p217+ tau peptides to the amount of total short tau peptides, before, during, or after the treatment. A decrease in values relative to baseline signals a positive response to treatment. Values can also increase temporarily in biological fluids as pathological tau is being cleared from the brain.

According to a particular aspect, the tauopathy includes, but is not limited to, one or more selected from the group consisting of Alzheimer's disease (including familial Alzheimer's disease and sporadic Alzheimer's disease), frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Preferably, the tauopathy is Alzheimer's disease (including familial Alzheimer's disease and sporadic Alzheimer's disease), FTDP-17 or progressive supranuclear palsy.

Most preferably, the tauopathy is Alzheimer's disease (including familial Alzheimer's disease and sporadic Alzheimer's disease).

According to one embodiment, a method of the invention comprises (i) contacting a biological sample, preferably a CSF sample, with a capture antibody directed against a p217+ tau epitope to capture p217+ tau peptides in the sample, (ii) contacting the captured p217+ tau peptides with a detection antibody directed against an epitope comprising amino acid residues 7 to 20 to thereby measure the amount of long p217+ tau peptides, and/or with a detection antibody directed against an epitope comprising amino acid residues 116 to 127 of tau protein to thereby measure the amount of long and short p217+ tau peptides in the sample, and (iii) determining whether or not the subject is suitable for an anti-p217+ tau antibody therapy based on the amount of the p217+ tau peptides or the ratio of the amount of short p217+ tau peptides to the amount of long p217+ tau peptides.

According to a particular aspect, it is determined that a subject is suitable for an anti-p217+ tau antibody therapy if the amount of p217+ tau peptides in the biological sample, or the ratio of the amount of short p217+ tau peptides to the amount of long p217+ tau peptides in the biological sample is significantly higher than a corresponding baseline value.

According to another particular aspect, a method of the invention comprises (i) contacting a biological sample, preferably a CSF sample, with a capture antibody directed against a p217+ tau epitope to capture p217+ tau peptides in the sample, or with a phosphorylation-independent capture antibody directed against a tau epitope between amino acids 150 and 250 of tau protein to capture total tau peptides in the sample, (ii) contacting the captured p217+ tau peptides, or the captured total tau peptides, with a detection antibody directed against an epitope comprising amino acid residues 116 to 127 of tau protein to thereby measure the amount of long and short p217+ tau peptides, or the amount of total short tau peptides, in the sample, and (iii) determining whether or not the subject is suitable for an anti-p217+ tau antibody therapy based on the amount of the ratio of the amount of short p217+ tau peptides to the amount of total short tau peptides in the biological sample.

According to one embodiment, it is determined that a subject is suitable for an anti-p217+ tau antibody therapy if the ratio of the amount of short p217+ tau peptides to the amount of total short tau peptides is significantly higher than a corresponding baseline value.

The invention also relates to measuring p217+ tau that is in complex with antibody in a biological sample as well as free p217+ tau in the sample that is not antibody-bound. In one embodiment, total antibody is captured using affinity techniques, followed by denaturing conditions including chaotrophs, heat-inactivation, or other protein disruption techniques. The p217+ tau is separated from antibody using rpHPLC, and is measured using methods of the invention, allowing for quantification of antibody-bound p217+ tau.

According to a general aspect, the invention relates to a method of monitoring a treatment with an anti-p217+ tau antibody in a subject, the method comprising: (i) obtaining a biological sample from the subject, (ii) separating the biological sample into an IgG enriched sample containing antibody-bound p217+ tau, and an IgG depleted sample containing antibody-free p217+ tau, (iii) purifying the p217+ tau away from IgGs by rpHPLC to obtain an antibody-free p217+ tau sample, (iv) contacting each of the IgG enriched sample and the antibody-free p217+ tau sample with a capture antibody directed against a p217+ tau epitope to capture p217+ tau peptides in each of the samples, (v) contacting the captured p217+ tau peptides in each of the samples with a detection antibody directed against an epitope comprising amino acid residues 7 to 20 to thereby measure the amount of long p217+ tau peptides, or with a detection antibody directed against an epitope comprising amino acid residues 116 to 127 of tau protein to thereby measure the amount of long and short p217+ tau peptides in each of the samples, (vi) calculating the ratio of the amount of antibody-bound p217+ tau to the amount of antibody-free p217+ tau, and (vii) monitoring the treatment with the anti-p217+ tau antibody in the subject based on the calculated ratio.

According to another general aspect, the invention relates to a method of monitoring a treatment with an anti-p217+ tau antibody in a subject, the method comprising: (i) obtaining a biological sample from the subject, (ii) obtaining a semi-denatured sample from the biological sample containing total p217+ tau, and obtaining a non-denatured sample from the biological sample containing antibody-free p217+ tau, wherein the semi-denatured sample is heated to denature the antibodies in the sample, (iii) contacting each of the semi-denatured sample and the non-denatured sample with a capture antibody directed against a p217+ tau epitope to capture p217+ tau peptides in each of the samples, (iv) contacting the captured p217+ tau peptides in each of the samples with a detection antibody directed against an epitope comprising amino acid residues 7 to 20 to thereby measure the amount of long p217+ tau peptides, or with a detection antibody directed against an epitope comprising amino acid residues 116 to 127 of tau protein to thereby measure the amount of long and short p217+ tau peptides in each of the samples, (v) calculating the amount of the antibody-bound p217+ tau in the sample by subtracting the amount of the antibody-free p217+ tau from the amount of the total p217+ tau, (vi) calculating the ratio of the antibody-bound p217+ tau to the antibody-free p217+ tau, and (vii) monitoring the treatment with the anti-p217+ tau antibody in the subject based on the calculated ratio.

According to a particular aspect, the effectiveness of the treatment in the subject is determined by monitoring the amount of the antibody-bound and antibody-free p217+ tau peptides before, during, or after the treatment. A decrease in values of antibody-free p217+ tau relative to baseline, or an increase in values of antibody-bound p217+ tau relative to baseline, and therefore an increase in the ratio of the antibody-bound p217+ tau to the antibody-free p217+ tau relative to baseline, signals a positive response to treatment. Values of antibody-free p217+ tau can also increase temporarily in biological fluids as pathological tau is being cleared from the brain.

According to particular aspects, the capture antibody of methods of the invention is conjugated to a bead, such as a magnetic bead. According to other particular aspects, the detection antibody is biotinylated.

According to particular aspects, the amount of p217+ tau peptides measured in methods of the invention can be determined using any suitable techniques known in the art, including ELISA and single molecule array platform. According to particular aspects, methods of the invention use a high sensitivity array platform, such as Quanterix Simoa or MSD S-plex, to measure the amount of p217+ tau peptides in a sample. According to a particular aspect, the lower limit of quantification of methods of the invention is about 40 fg/ml and the lower limit of detection of the method is about 2 fg/ml.

According to a particular aspect, the sample used in methods of the invention is a biological sample, such as a blood, brain homogenate, or cerebral spinal fluid (CSF) sample. Preferably, the sample is a CSF sample. According to a particular aspect, the sample is a crude CSF sample. According to another particular aspect, the sample is obtained after fractionating a biological sample, such as CSF, using reverse phase high-performance liquid chromatography (rpHPLC), which separates full-length tau protein and differentially-sized tau fragments.

According to a particular aspect, the capture antibody of methods of the invention comprises immunoglobulin heavy chain HCDR1, HCDR2 and HCDR3 having the polypeptide sequences of SEQ ID NOs: 32, 33 and 34, respectively, and immunoglobulin light chain LCDR1, LCDR2 and LCDR3 having the polypeptide sequences of SEQ ID NOs: 35, 36 and 37, respectively. Preferably, the capture antibody is a pT3 antibody comprising heavy chain variable region comprising polypeptide sequence of SEQ ID NO: 28 and a light chain variable region having the polypeptide sequence of SEQ ID NO: 29.

According to a particular aspect, the detection antibody of methods of the invention comprises immunoglobulin heavy chain HCDR1, HCDR2 and HCDR3 having the polypeptide sequences of SEQ ID NOs: 2, 3 and 4, respectively; and immunoglobulin light chain LCDR1, LCDR2 and LCDR3 having the polypeptide sequences of SEQ ID NOs: 5, 6 and 7, respectively. Preferably, the detection antibody is a pT82 antibody comprising a heavy chain variable region having the polypeptide sequence of SEQ ID NO: 8 and a light chain variable region having the polypeptide sequence of SEQ ID NO: 9.

According to another particular aspect, the detection antibody of methods of the invention comprises immunoglobulin heavy chain HCDR1, HCDR2 and HCDR3 having the polypeptide sequences of SEQ ID NOs: 12, 13 and 14, respectively; and immunoglobulin light chain LCDR1, LCDR2 and LCDR3 having the polypeptide sequences of SEQ ID NOs: 15, 16 and 17, respectively. Preferably, the detection antibody is a hT43 antibody comprising a heavy chain variable region having the polypeptide sequence of SEQ ID NO: 18 and a light chain variable region having the polypeptide sequence of SEQ ID NO: 19.

### Kits

In another general aspect, the invention relates to a kit comprising (a) a capture antibody directed against a p217+ tau epitope, optionally a phosphorylation-independent capture antibody directed against a tau epitope between amino acids 150 and 250 of tau protein, and (b) at least one detection antibody directed against a tau protein epitope comprising amino acid residues 7 to 20 or 116 to 127 of tau protein. The kit is used to measure the amount of p217+ tau peptides, which is used the ratio of the amount of short p217+ tau peptides to the amount of long p217+ tau peptides, and/or the ratio of the amount of short p217+ tau peptides to the amount of total short tau peptides in a sample.

The detection antibody can contain any detectable label (e.g., fluorescent molecule, biotin, etc.) which is directly detectable or detectable via a secondary reaction (e.g., reaction with streptavidin). Alternatively, a second reagent containing the detectable label can be used, where the second reagent has binding specificity for the primary antibody. In a diagnostic kit suitable for measuring p217+ tau in a biological sample, the antibodies of the kit can be supplied pre-bound to a solid phase, such as to the wells of a microtiter dish or to beads.

According to a particular aspect, the capture antibody of a kit of the invention comprises immunoglobulin heavy chain HCDR1, HCDR2 and HCDR3 having the polypeptide sequences of SEQ ID NOs: 32, 33 and 34, respectively, and immunoglobulin light chain LCDR1, LCDR2 and LCDR3 having the polypeptide sequences of SEQ ID NOs: 35, 36 and 37, respectively. Preferably, the capture antibody is a pT3 antibody comprising heavy chain variable region comprising polypeptide sequence of SEQ ID NO: 28 and a light chain variable region having the polypeptide sequence of SEQ ID NO: 29.

According to a particular aspect, the detection antibody of a kit of the invention comprises immunoglobulin heavy chain HCDR1, HCDR2 and HCDR3 having the polypeptide sequences of SEQ ID NOs: 2, 3 and 4, respectively; and immunoglobulin light chain LCDR1, LCDR2 and LCDR3 having the polypeptide sequences of SEQ ID NOs: 5, 6 and 7, respectively. Preferably, the detection antibody is a pT82 antibody comprising a heavy chain variable region having the polypeptide sequence of SEQ ID NO: 8 and a light chain variable region having the polypeptide sequence of SEQ ID NO: 9.

According to another particular aspect, the detection antibody of a kit of the invention comprises immunoglobulin heavy chain HCDR1, HCDR2 and HCDR3 having the polypeptide sequences of SEQ ID NOs: 12, 13 and 14, respectively; and immunoglobulin light chain LCDR1, LCDR2 and LCDR3 having the polypeptide sequences of SEQ ID NOs: 15, 16 and 17, respectively. Preferably, the detection antibody is a hT43 antibody comprising a heavy chain variable region having the polypeptide sequence of SEQ ID NO: 18 and a light chain variable region having the polypeptide sequence of SEQ ID NO: 19.

According to another particular aspect, a kit of the invention is used to measure the amount of p217+ tau peptides, the ratio of the amount of short p217+ tau peptides to the amount of long p217+ tau peptides, and/or the ratio of the amount of short p217+ tau peptides to the amount of total short tau peptides in a sample using a method of the invention.

The contents of all cited references (including literature references, issued patents, published patent applications, and co-pending patent applications) cited throughout this application are hereby expressly incorporated by reference.

### EMBODIMENTS

The invention provides also the following non-limiting embodiments.

Embodiment 1 is a method of measuring the amount of p217+ tau peptides in a sample, comprising:
(i) contacting the sample with a capture antibody directed against a p217+ tau epitope to capture p217+ tau peptides in the sample, and
(ii) contacting the captured p217+ tau peptides with a detection antibody directed against an epitope comprising amino acid residues 119 to 126, such as amino acid residues 116-127, of tau protein, or an epitope containing amino acid residues 7 to 20 of tau protein to thereby measure an amount of p217+ tau peptides or an amount of long p217+ tau peptides, respectively.

Embodiment 2 is a method of determining a relative amount of long p217+ tau peptides or short p217 tau peptide fragments in a sample, comprises
(i) contacting the sample with a capture antibody directed against a p217+ tau epitope to capture p217+ tau peptides in the sample,
(ii) contacting the captured p217+ tau peptides with a first detection antibody directed against an epitope comprising amino acid residues 119 to 126 of tau protein to thereby measure an amount of p217+ tau peptides,
(iii) contacting the captured p217+ tau peptides with a second detection antibody directed against an epitope comprising amino acid residues 7 to 20 of tau protein to thereby measure an amount of long p217+ tau peptides, and
(iv) determining a relative amount of long p217+ tau peptides or short p217+ tau peptides based on the amount of p217+ tau peptides and the amount of long p217+ tau peptides.

Embodiment 3 is the method of Embodiment 1 or 2, wherein the capture antibody is conjugated to a bead, and wherein the detection antibody is biotinylated.

Embodiment 4 is the method of any of Embodiments 1-3, wherein the amount of p217+ tau peptides in the sample is measured using a high sensitivity platform.

Embodiment 5 is the method of any of Embodiments 1-4, wherein the lower limit of quantification of the method is about 40 fg/ml of the p217+ tau peptides and the lower limit of detection of the method is about 2 fg/ml of the p217+ tau peptides.

Embodiment 6 is the method of any of Embodiments 1-5, wherein the sample is a biological sample, preferably a CSF sample, from a subject, and the method further comprises determining whether or not the subject suffers from a tauopathy or is at risk of developing a tauopathy based on the amount of the p217+ tau peptides, the ratio of the amount of short p217+ tau peptides to the amount of long p217+ tau peptides, or the ratio of the amount of short p217+ tau peptides to the amount of total short tau peptides in the biological sample.

Embodiment 7 is the method of Embodiment 6, wherein the subject is determined to suffer from a tauopathy or to be at risk of developing a tauopathy if the amount of the p217+ tau peptides in the biological sample, the ratio of the amount of short p217+ tau peptides to the amount of long p217+ tau peptides, or the ratio of the amount of short p217+ tau peptides to the amount of total short tau peptides is significantly higher than a corresponding baseline values, such as the mean corresponding value of Healthy Volunteers.

Embodiment 8 is the method of any of Embodiments 1-5, wherein the sample is a biological sample, preferably a CSF sample, from a subject under a treatment of a tauopathy, and the method further comprises determining the effectiveness of the treatment in the subject based on the amount of the p217+ tau peptides, the ratio of the amount of short p217+ tau peptides to the amount of long p217+ tau peptides, or the ratio of the amount of short p217+ tau peptides to the amount of total short tau peptides in the biological sample.

Embodiment 9 is the method of Embodiment 8, wherein the treatment is determined to be effective if the amount of the p217+ tau peptides in the biological sample decreases over the course of treatment.

Embodiment 10 is the method of any of Embodiments 6-9, wherein the tauopathy is selected from the group consisting of Alzheimer's disease (including familial Alzheimer's disease and sporadic Alzheimer's disease), frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

Embodiment 11 is the method of Embodiment 10, wherein the tauopathy is Alzheimer's disease.

Embodiment 12 is the method of any of Embodiments 1-5, wherein the sample is a biological sample, preferably a CSF sample, from a human subject, and the method further comprises determining whether or not the subject is suitable for an anti-p217+ tau antibody therapy based on the amount of the p217+ tau peptides, the ratio of the amount of short p217+ tau peptides to the amount of long p217+ tau peptides, or the ratio of the amount of short p217+ tau peptides to the amount of total short tau peptides in the biological sample.

Embodiment 13 is the method of Embodiment 12, wherein the subject is determined to be suitable for anti-p217+ tau antibody therapy if the amount of the p217+ tau peptides in the biological sample, the ratio of the amount of short p217+ tau peptides to the amount of long p217+ tau peptides, or the ratio of the amount of short p217+ tau peptides to the amount of total short tau peptides is significantly higher than a corresponding baseline values, such as the mean corresponding value of Healthy Volunteers.

Embodiment 14 is a method of monitoring a treatment with an anti-p217+ tau antibody in a subject, the method comprising:
i. obtaining a biological sample from the subject,
ii. separating the biological sample into an IgG enriched sample containing antibody-bound p217+ tau, and an IgG depleted sample containing antibody-free p217+ tau,
iii. contacting each of the IgG enriched sample and the IgG depleted sample with a capture antibody directed against an epitope comprising phosphorylated T212 and/or phosphorylated T217 of the tau protein to capture the p217+ tau peptides in each of the samples,
iv. contacting the captured p217+ tau peptides with a detection antibody directed against an epitope comprising amino acid residues 7 to 20 or 116 to 127 of tau protein to thereby measure the amount of the antibody-bound p217+ tau and the amount of the antibody-free p217+ tau in the biological sample,
v. calculating the ratio of the antibody-bound p217+ tau to the antibody-free p217+ tau, and
vi. monitoring the treatment with the anti-p217+ tau antibody in the subject based on the calculated ratio.

Embodiment 15 is a method of monitoring a treatment with an anti-p217+ tau antibody in a subject, the method comprising:
i. obtaining a biological sample from the subject,
ii. obtaining a semi-denatured sample from the biological sample containing total p217+ tau, and obtaining a non-denatured sample from the biological sample containing antibody-free p217+ tau, wherein the semi-denatured sample is heated to denature the antibodies in the sample,
iii. contacting each of the semi-denatured sample and the non-denatured sample with a capture antibody directed against an epitope comprising phosphorylated T212 and/or phosphorylated T217 of the tau protein to capture the p217+ tau peptides in each of the samples,
iv. contacting the captured p217+ tau peptides with a detection antibody directed against an epitope comprising amino acid residues 7 to 20 or 116 to 127 of tau protein to thereby measure the amount of the total p217+ tau and the amount of the antibody-free p217+ tau in the biological sample,
v. calculating the amount of the antibody-bound p217+ tau in the sample by subtracting the amount of the antibody-free p217+ tau from the amount of the total p217+ tau,
vi. calculating the ratio of the antibody-bound p217+ tau to the antibody-free p217+ tau, and
vii. monitoring the treatment with the anti-p217+ tau antibody in the subject based on the calculated ratio.

Embodiment 16 is the method of any one of Embodiments 1 to 15, wherein the capture antibody comprises immunoglobulin heavy chain HCDR1, HCDR2 and HCDR3 having the polypeptide sequences of SEQ ID NOs: 32, 33 and 34, respectively, and immunoglobulin light chain LCDR1, LCDR2 and LCDR3 having the polypeptide sequences of SEQ ID NOs: 35, 36 and 37, respectively; preferably, the capture antibody has a heavy chain variable region comprising polypeptide sequence of SEQ ID NO: 28 and a light chain variable region having the polypeptide sequence of SEQ ID NO: 29.

Embodiment 17 is the method of any one of Embodiments 1 to 16, wherein the detection antibody comprises immunoglobulin heavy chain HCDR1, HCDR2 and HCDR3 having the polypeptide sequences of SEQ ID NOs: 2, 3 and 4, respectively; and immunoglobulin light chain LCDR1, LCDR2 and LCDR3 having the polypeptide sequences of SEQ ID NOs: 5, 6 and 7, respectively; preferably, the detection antibody comprises a heavy chain variable region having the polypeptide sequence of SEQ ID NO: 8 and a light chain variable region having the polypeptide sequence of SEQ ID NO: 9.

Embodiment 18 is the method of any one of Embodiments 1 to 16, wherein the detection antibody comprises immunoglobulin heavy chain HCDR1, HCDR2 and HCDR3 having the polypeptide sequences of SEQ ID NOs: 12, 13 and 14, respectively; and immunoglobulin light chain LCDR1, LCDR2 and LCDR3 having the polypeptide sequences of SEQ ID NOs: 15, 16 and 17, respectively; preferably, the detection antibody comprises a heavy chain variable region having the polypeptide sequence of SEQ ID NO: 18 and a light chain variable region having the polypeptide sequence of SEQ ID NO: 19.

Embodiment 19 is the method of any one of Embodiments 1 to 18, wherein the sample is a blood, brain homogenate, or cerebral spinal fluid (CSF) sample.

Embodiment 20 is the method of any one of Embodiments 1 to 19, wherein the sample is obtained after fractionating a biological sample using reverse phase high-performance liquid chromatography (rpHPLC).

Embodiment 21 is an isolated detection antibody or antigen-binding fragment thereof that binds to a tau protein at an epitope comprising amino acid residues 116 to 127 of tau protein, comprising:
a. immunoglobulin heavy chain HCDR1, HCDR2 and HCDR3 having the polypeptide sequences of SEQ ID NOs: 2, 3 and 4, respectively; and
b. immunoglobulin light chain LCDR1, LCDR2 and LCDR3 having the polypeptide sequences of SEQ ID NOs: 5, 6 and 7, respectively.

Embodiment 22 is the isolated detection antibody or antigen-binding fragment of Embodiment 21 comprising a heavy chain variable region having a polypeptide sequence of SEQ ID NO: 8, and a light chain variable region having a polypeptide sequence of SEQ ID NO: 9, preferably.

Embodiment 23 is an isolated detection antibody or antigen-binding fragment thereof that binds to a tau protein at an epitope comprising amino acid residues 7 to 20 of tau protein, comprising:
a. immunoglobulin heavy chain HCDR1, HCDR2 and HCDR3 having the polypeptide sequences of SEQ ID NOs: 12, 13 and 14, respectively; and
b. immunoglobulin light chain LCDR1, LCDR2 and LCDR3 having the polypeptide sequences of SEQ ID NOs: 15, 16 and 17, respectively.

Embodiment 24 is the isolated detection antibody or antigen-binding fragment of Embodiment 23 comprising a heavy chain variable region having a polypeptide sequence of SEQ ID NO: 18, and a light chain variable region having a polypeptide sequence of SEQ ID NO: 19, preferably.

Embodiment 25 is an isolated nucleic acid encoding the detection antibody or antigen-binding fragment thereof of any of Embodiments 21-24.

Embodiment 26 is a vector comprising the nucleic acid of Embodiment 25.

Embodiment 27 is a host cell comprising the nucleic acid of Embodiment 25.

Embodiment 28 is a method of producing the detection antibody or antigen-binding fragment thereof of any one of Embodiments 21-24, comprising culturing a cell comprising a nucleic acid encoding the antibody or antigen-binding fragment under conditions to produce the antibody or antigen-binding fragment, and recovering the antibody or antigen-binding fragment from the cell or cell culture.

Embodiment 29 is a kit comprising:
a. a capture antibody directed against a singly- or multiply-phosphorylated tau protein epitope comprising phosphorylated T212 and/or phosphorylated T217 of the tau protein, and
b. a detection antibody directed against a tau protein epitope comprising amino acid residues 7 to 20 or 116 to 127 of tau protein;
wherein the kit is used to measure the amount of p217+ tau peptides in a sample.

Embodiment 30 is the kit of Embodiment 29, wherein the capture antibody comprises immunoglobulin heavy chain HCDR1, HCDR2 and HCDR3 having the polypeptide sequences of SEQ ID NOs: 32, 33 and 34, respectively, and immunoglobulin light chain LCDR1, LCDR2 and LCDR3 having the polypeptide sequences of SEQ ID NOs: 35, 36 and 37, respectively; preferably, the capture antibody has a heavy chain variable region comprising polypeptide sequence of SEQ ID NO: 28 and a light chain variable region having the polypeptide sequence of SEQ ID NO: 29.

Embodiment 31 is the kit of Embodiment 29 or 30, wherein the detection antibody is the isolated detection antibody of any one of Embodiments 20-23.

### EXAMPLES

The following examples of the invention are to further illustrate the nature of the invention. It should be understood that the following examples do not limit the invention and that the scope of the invention is to be determined by the appended claims.

### Example 1. High Sensitivity Assay for Detecting p217+ Tau

Assay-specific reagents were as follows: Simoa Homebrew kit (Quanterix, cat #101351), Helper beads (Quanterix, cat #101732), pT3 mouse monoclonal antibody (mAb), hT43 mAb, pT82 mAb and hT7 mAb. pT3 is the parental antibody developed at Janssen that recognizes p217+ tau, and the humanized version thereof is referred to herein as humanized pT3 mAb.

The samples were diluted in 50 mM Tris, 50 mM NaCl, 5 mM EDTA, 2% Bovine Serum Albumin, 0.1% Tween 20, 0.05% ProClin 300, pH7.8.

Three custom peptides made by New England Peptide were used to calibrate the assay (calibrant peptides).

Peptide pT3xhT43 contains hT43, PT51 and pT3 epitopes connected by PEG4 linkers and has a molecular weight of 6893 g/mol. The amino acid sequence of peptide pT3xhT43 is PRQEFEVMEDHAGTYGLGDR(dPEG4)GKTKIATPRGAAPPGQKG(dPEG4)GSRSR(pT)PS LP(pT)PPTREPKKV-amide (SEQ ID NO: 22).

Peptide pT3xpT82 contains pT82 and pT3 epitopes connected by a PEG4 linker and has a molecular weight of 4551 g/mol. The amino acid sequence of peptide pT3xpT82 is Ac-SLEDEAAGHVTQARMVSK(dPEG4)GSRSR(pT)PSLP(pT)PPTREPKKV-amide (SEQ ID NO: 23).

Peptide hT7xpT82 contains pT82 and hT7 epitopes connected by a PEG4 linker and has a molecular weight of 3619 g/mol. The amino acid sequence of peptide hT7xpT82 is Ac-SLEDEAAGHVTQARMVSK(dPEG4)PRGAAPPGQKGQANA-amide (SEQ ID NO: 24).

### Reagent Prep

The capture beads were coated with 0.3 mg/ml capture Ab following the protocol provided in the Quanterix manual. The coated capture beads were diluted in Bead Diluent Buffer to 200,000 beads/ml, and 200,000 beads/ml Helper Beads were added so that the total concentration of beads was 400,000 beads/ml.

The detection antibodies were biotinylated at 60× following the protocol provided in the Quanterix manual and were diluted in Homebrew Detector/Sample Diluent to 1.8 ug/ml.

The calibrant peptides were reconstituted to 5 mg/ml in 0.1% phosphoric acid/water, aliquoted to 20 ul and frozen. When ready for use, the calibrant peptide aliquots were thawed and diluted 1:1000 (e.g. 1.5 ul into 1498.5 ul), and the dilutions were diluted 1:1000 so that the final concentration of the peptides was 5000 pg/ml. A standard curve with 3× jumps was made, starting at 30 pg/ml.

CSF samples were diluted at least 1:4 in Sample Diluent. Healthy volunteer (HV) samples were diluted 1:5 or 1:10, and AD samples were diluted at least 1:20.

### Simoa Assay

A custom Simoa assay was created comprising a two step protocol comprising 35 minutes with capture Ab, sample, and detection Ab, and washing, followed by 5 minutes with streptavidin β-galactosidase (SBG). Each reaction comprised 25 ul beads solution, 100 ul sample or calibrant, 20 ul detection solution, 100 ul SBG. The antibodies were assigned names, and up to five capture antibodies and five detection antibodies could be loaded at a time. The reactions were performed in the Simoa cuvettes by the instrument, washed one last time, and loaded into measurement discs with β-galactosidase substrate (RGP) before measurements were taken by the instrument.

### Example 2. Separation of Native Tau Fragments on rpHPLC

Reagents were as follows: Trifluoracetic acid (HPLC grade), Water (HPLC grade), Acetonitrile (HPLC grade), Phosphoric acid (analytical grade), and HPLC binary gradient system, Immunoassay Buffer (100 mM TrisHCl, 100 mM NaCl, 0.05% Tween, & BSA, pH7.8).

The protocol was as follows: 500 ul of frozen CSF was thawed on ice for 30 min. The thawed CSF was added to 1.5 ml of 100 mM sodium phosphate pH 2.5 containing 100 mM sodium chloride and mixed. 1.8 ml of the resulting mixture was applied onto a C18 or similar reverse phase chromatography column equilibrated in 0.1% trifluoracetic acid in water. The HPLC column was then developed in an increasing gradient of acetonitrile. Fractions were collected across the elution. The fractions were adjusted to 10 mM in guanidine HCl and then dried in a vacuum concentrator. The dried fractions were resuspended in immunoassay buffer and subjected to measurement of the tau peptide in the fraction based on an anti-tau capture and detection antibody pair of the invention.

### Example 3. Quantification of p217+ Tau that is Free or Bound by Antibodies

With additional upstream sample manipulation, the high sensitivity pT3-based assays can be used to measure the binding of p217+ tau by antibodies that are either produced within a patient or are administered exogenously, e.g. humanized pT3 mAb. This technique can be used as a pharmacodynamic assay to study therapeutic anti-p217+ tau antibodies such as humanized pT3 mAb. For example, the following methods can be used to measure p217+ tau that is antibody-free vs. antibody-bound.

Assay 1: Quantification of Free Vs. Bound p217+ Tau in Biological Fluids Using Immunocapture Depletion Followed by rpHPLC

Biological fluid (e.g. CSF) was incubated with protein A/G-coated magnetic beads (15 μl bead slurry per 0.5 mL CSF) for 2 hrs with rocking at room temperature to capture immunoglobulins in the sample. Beads were precipitated by magnet and the supernatant was transferred to second tube (sample=“IgG depleted supernatant”). The beads were washed 4× with 1 mL cold Phosphate Buffered Saline (PBS). 0.5 mL of 6M GuHCl was then added to the tubes containing (a) washed beads and (b) IgG depleted supernatant, and the tubes were incubated for 20 minutes with rocking at room temperature. Beads were then precipitated by magnet, and the resulting supernatant was transferred to a third tube (sample=“IgG concentrated supernatant”). Finally, 0.1 M phosphoric acid (pH 2) was added to the two solutions (1.0 mL phosphoric acid was added to the denatured IgG depleted supernatant, and 1.5 mL phosphoric acid was added to the IgG concentrated supernatant, to make samples up to a final volume of 2.0 mL) prior to separation by rpHPLC, carried out as in Example 2. The resulting rpHPLC fractions were reconstituted as described in Example 2 and measured using the Simoa p217+ tau assays of Example 1. Signal from the IgG depleted supernatant represents free p217+ tau (i.e., that which is not bound by antibodies), while signal from the IgG concentrated supernatant represents bound p217+ tau (i.e., that which is bound by antibodies, such as humanized pT3 mAb). rpHPLC separation and Simoa p217+ measurement of the same parental biological fluid (e.g. CSF) that was not subjected to the immunocapture/depletion process was analyzed simultaneously for assessment of total p217+ tau signal, as a control or as a normalizer for the free and bound measurements.

Assay 2: Quantification of Free Vs. Bound p217+ Tau in Biological Fluids Using Heat Denaturation of Antibodies

An aliquot of biological fluid of interest (e.g. CSF) was heated at 95° C. for 4 minutes, followed by chilling on wet ice for 4 minutes (sample=“semi-denatured fluid”). In parallel, a second aliquot of the same fluid was chilled on wet ice for 8 minutes (sample=“non-denatured fluid”). Both samples were then measured using the Simoa p217+ tau assays of Example 1. Semi-denatured fluid signal represents total p217+ tau, while non-denatured fluid represents free p217+ tau. Subtracting the latter from the former yields measurement of bound tau. The precise heating time and temperature was determined to irreversibly modify any antibodies in the fluid such that they could no longer interfere with the Simoa p217+ tau assays, while the p217+ tau signal itself was spared any impact. This assay is not a direct measure of whether the antibodies are bound to p217+ tau, rather it demonstrates that assay-competing antibodies are present. However, the assay yielded similar results to those of the more laborious Assay 1.

### Example 4. Biological Samples

### Samples Used for Assay Development and Technical Qualification

The assays of Examples 1-3 were developed using CSF pooled from human subjects with high tau levels. Some experiments were also performed with CSF pooled from human subjects with low tau levels to ensure assay sensitivity that would be required for testing of healthy volunteers in Phase 1 trials. CSF from Cynomolgus Macaque (Macaca fascicularis) and Common Marmoset (Callithrix jacchus), obtained from Neu Encepharm GmbH (animal testing CRO), was also measured using the assays of Examples 1-3. Additionally, flash frozen brain samples from cognitively normal human subjects and Common Marmoset were homogenized and measured using the assays of Examples 1 and 3. Some experiments were performed with individual serums from clinically defined HV and AD subjects.

### Samples Used for Preliminary Clinical Qualification

Cohort 1 (“Interassay correlation cohort”): Ventricular fluid (VF) and lumbar fluid (LF) CSF samples were obtained from subjects with Normal Pressure Hydrocephalus (NPH) (n=11) (University of Kuopio, Professor Ville Lenoinen). These samples were separated by CSF Aβ42, total tau (tTau), and pTau181 measurements as determined by Innotest assays performed at the University of Sahlgrenska (Professor Kaj Blennow), and by brain biopsy amyloid and tau immunohistochemistry (IHC) measurements. rpHPLC and Simoa p217+ tau measurements were performed at Janssen Neuroscience Biomarkers, La Jolla.

Cohort 2 (“clear HV vs. clear AD cohort”): LF CSF samples from biochemically defined Alzheimers Disease (AD) vs. Healthy Volunteer (HV) subjects (n=20 per group) were obtained from University of Sahlgrenska (Professor Kaj Blennow). CSF Aβ42, tTau, and pTau181 measurements by Innotest assays were performed at University of Sahlgrenska. Samples were selected from a large panel of samples based on segregation into predetermined AD vs. HV cutoff measures (AD=CSF Aβ42<400 pg/ml AND CSF tTau>600 pg/ml, HV=CSF Aβ42>400 pg/ml AND CSF tTau<600 pg/ml). rpHPLC and Simoa p217+ tau measurements were performed at Janssen Neuroscience Biomarkers, La Jolla.

Cohort 3 (“HV vs. ARAD vs. early stage AD cohort”): LF CSF samples from clinically defined normal (Clinical Dementia Rating 0; CDR 0) vs. mild memory complaint (CDR 0.5) subjects (n=20 per group) were obtained from Janssen study ALZ1005/1002. CSF Aβ42, tTau, and pTau181 measurements by Innotest assays were performed at University of Sahlgrenska. Based on the CDR and CSF Aβ42 scores, the subjects were classified into (a) HV=CDR 0 and Aβ42>600 pg/ml, (b) At Risk of AD (ARAD)=CDR 0 and Aβ42<600 pg/ml, (c) potentially non-AD dementia=CDR 0.5 and Aβ42>600 pg/ml, and (d) early stage AD=CDR 0.5 and Aβ42<600 pg/ml. rpHPLC and Simoa p217+ tau measurements were performed at Janssen Neuroscience Biomarkers, La Jolla.

Cohort 4 (“CDR 0 vs CDR1 cohort”): LF CSF samples from clinically defined normal (Clinical Dementia Rating 0; CDR 0) vs. mild memory complaint (CDR 1) subjects (n=5 per group) were obtained from Washington University. CDR & MMSE, as well as CSF Aβ42, tTau, and pTau181 measurements by Innotest assays were obtained at Washington University. Prior to shipment samples were coded so that Janssen was blind to sample identity or characterization. rpHPLC and Simoa tTau & p217+ tau measurements were performed at Janssen Neuroscience Biomarkers, La Jolla and sent to Washington University for analysis.

Cohort 5 (“HV vs. MCI vs. AD cohort”): LF CSF samples from clinically and biochemically (Innotest AB42>600 pg/ml) defined HV (n=7) were obtained from Precision Medicine, San Diego. LF CSF samples from clinically and biochemically (Innotest AB42<600 pg/ml) defined MCI (n=28) and AD (n=12) were obtained from University of Antwerp. rpHPLC and Simoa p217+ tau measurements were performed at Janssen Neuroscience Biomarkers, La Jolla.

Cohort 6 (“Disease severity and progression cohort”): LF CSF samples from clinically defined AD (Clinical Dementia Rating 1+) subjects (n=235) were obtained from Janssen study ELN115727301/302. These samples were baseline (pre-dose) samples from all subjects in the trial. In addition, CSF samples from 78 week follow up on the placebo subjects (n=90) was included to evaluate biomarkers of disease progression. Cognitive assessment (ADAS-COG, MMSE, NTB, CDR.SOB), ApoE genotype, gender, & age were obtained from the trial. Innotest AB42, Innotest AB40, Simoa NFL, pT3xpT82, pT3xhT43, and hT7xpT82 assays were performed at Janssen Neuroscience Biomarkers, La Jolla. Subjects were confirmed amyloid positive or negative based on the AB42/40 ratio cutoff of 0.09 (e.g. subjects with ratio<0.09=amyloid positive=AD, while those >0.09=amyloid negative=dementia from non-AD cause. 27 of the 235 subjects were determined to be amyloid negative, both groups were analyzed separately.

Samples Used for Evaluation of Target Engagement after Treatment with Anti-p217+ Agents

LF CSF from HV subjects (n=40) treated with placebo or JNJ63733657 (single IV injection) were obtained from Janssen trial JNJ63733657EDI1001. pT3xpT82 assays were performed at Janssen Neuroscience Biomarkers, La Jolla. pT3xhT43 assays were performed at Quanterix Corporation, Lexington MA.

### Example 5. Screening of Capture and Detection Antibody Pairs Using the Simoa Platform

Prior reports from Janssen Neuroscience Discovery and the literature (e.g., Meredith et al. PLoS One. 8(10):e76523, 2013; Barthelemy et al., J Alzheimers Dis. 51(4):1033-43, 2016; Russell et al., J Alzheimers Dis. 55(1):303-313, 2017; Hanger et al. J Biol Chem. 282(32):23645-54, 2007) have indicated that tau fragments containing amino acids 200-220, and especially some combination of phosphorylation at amino acids 212, 214, 217, are enriched in AD. Developing an assay to measure this particular tau species (“p217+ tau”) could thus yield an improved biomarker for AD diagnosis and/or staging, as well as a potential predictive and/or pharmacodynamic assay for new drugs targeting this tau moiety. However, tau can be present at low levels (<200 pg/ml) in healthy volunteers, and p217+ tau is a minority component of total tau, so p217+ tau assays require optimal antibody pairs and high sensitivity.

To achieve this goal, a set of anti-tau mAbs discovered at Janssen, as well as some high-affinity commercial anti-tau mAbs, were evaluated for their ability to yield signal in a sandwich ELISA (sELISA) format when paired with pT3. Antibody pairs were screened on the Simoa HD-1 Analyzer platform (Quanterix Corporation) to provide the sensitivity required, using a serial dilution of a pool of CSF from AD subjects. Assay performance was based on Signal/Noise=average enzymes per bead (AEB) of sample diluted in sample diluent/AEB of assay diluent alone. Optimal detection antibodies to pair with pT3 were hT43, pT82, the Quanterix tau 2.0 detector reagent, and BT2 in that order of sensitivity (Table 1). hT43 and the Quanterix tau 2.0 detector reagent recognize the N-terminal region of tau, while pT82 and BT2 recognize sequences closer to the mid-region of tau. The best N-terminal (hT43) and mid-region (pT82) mAbs were selected for further optimization. Screening was performed in parallel at Janssen Neuroscience Biomarkers and at Quanterix Corporation, yielding similar results.

TABLE 1
Screening of specificity in AD CSF of tau detection antibodies paired with pT3.

| | | S/N @ | S/N @ | S/N @ | S/N @ | S/N @ | S/N @ |
|---|---|---|---|---|---|---|---|
| Detection | Epitope | 1:2 | 1:4 | 1:8 | 1:16 | 1:32 | 1:64 |
| Antibody | (tau aa) | dilution | dilution | dilution | dilution | dilution | dilution |
| hT43 | 7-20 | 955 | 587 | 313 | 183 | 102 | 55 |
| Quanterix tau | 16-24 | 525 | 262 | 147 | 75 | 35 | 19 |
| 2.0 assay | | | | | | | |
| detector | | | | | | | |
| pT82 | 116-127 | 733 | 400 | 211 | 105 | 48 | 23 |
| PT51 | 151-158 | 165 | 52 | 17 | 6 | 3 | 1 |
| PT98 | 159-163 | NT | NT | 17 | 7 | 3 | 2 |
| pT89 | 166-182 | 87 | 24 | 9 | 4 | 2 | 1 |
| BT2 | 193-198 | 732 | 245 | 78 | 28 | 10 | 4 |
| HT52 | 393-398 | NT | NT | 1 | 1 | 1 | 1 |
| HT60 | 423-440 | NT | NT | 1 | 1 | 1 | 1 |

Antibody epitope on tau and Signal/Noise (S/N) ratios in measuring pooled CSF from AD subjects is shown; NT = not tested.

### Example 6. Optimization of pT3xhT43 and pT3xpT82 Assays

A series of optimization experiments were performed based on general Quanterix experience with optimizing assays on the Simoa platform. 10% mouse serum or 500 μg/ml mouse IgG were added to the detector diluents but did not improve assay sensitivity. Titrations of Detector mAb concentration (0.15, 0.3, 0.6, 1.2, and 1.8 μg/ml), SOG concentration (100, 200, or 300 pM), and Capture mAb bead concentrations (300K/well, 150K+200K helper beads) were evaluated. Protocol incubation times (65 minute vs. 35 minute), and sample volume (100 vs. 150 μl) were also evaluated. The ideal reagent concentrations for both assays were 150K capture beads+200K helper beads, 1.8 pg/ml detector, and 200 pM SBG, respectively. Sample volume and incubation time had minimal impact on the assay, so the lower conditions of 100 ul sample and 35 minute incubation were chosen.

### Example 7. Technical Qualification of pT3xhT43 and pT3xpT82 Assays

Linear Range with Calibrant Material

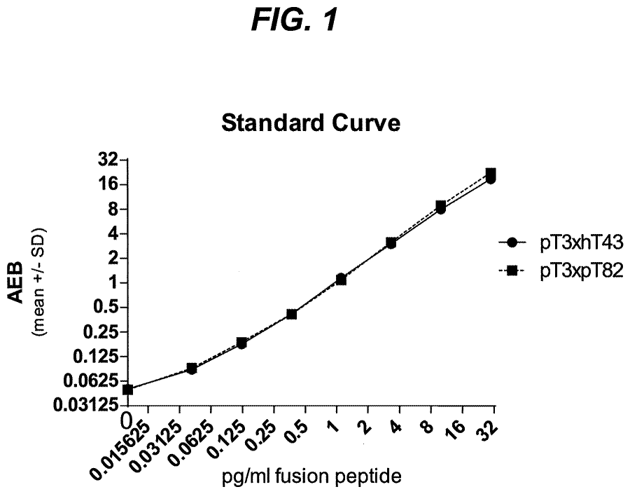

Calibrant peptides described in Example 1 were produced. The calibrant peptides contained the core epitopes of pT3 and hT43, or pT3 and pT82 separated by PEG4 linkers, and they were used to generate standard curves. A representative standard curve is shown in FIG. 1. Calibrant peptides were titrated from 30 pg/ml to 0.041 pg/ml in 1:3 jumps in assay buffer and measured with the pT3xhT43 and pT3xpT82 assays. A 4-parameter curve fit data reduction method (4PL, 1/y2 weighted) was used to generate the calibration curve. The lower limit of detection (LLOD) was defined as the calculated calibrant level yielding an AEB equal to the average of the zero calibrator+2.5 standard deviations (SD), including 10% coefficient of variation (CV). With these criteria, the representative data yielded an LLOD of ˜0.002 pg/ml. The Linear Range of the assay, lower limit of quantification (LLOQ) and upper limit of quantification (ULOQ), was defined as the lowest and highest standard curve points achieving CV<20% and recovery 80-120% of expected. With these criteria, the linear range for both the pT3xhT43 and pT3xpT82 assays was 0.041 to 30 pg/ml (FIG. 1, Table 2).

TABLE 2
Representative calibrant curve for optimized
pT3xhT43 assay, with LLOD calculations

| pg/ml | Avg | | CV | |
|---|---|---|---|---|
| calibrant | AEB | SD | (%) | S/B |
| 0 | .0185 | .0019 | 10 | |
| .034 | .1369 | .0260 | 19 | 7 |
| .103 | .1952 | .0039 | 2 | 11 |
| .309 | .5221 | .0626 | 12 | 28 |
| .926 | 1.4545 | .0145 | 1 | 78 |
| 2.78 | 5.4789 | .1096 | 2 | 295 |
| 8.33 | 11.4588 | 1.0313 | 9 | 617 |
| 25 | 24.0576 | .2406 | 1 | 1297 |

LLOD = .0185 + (2.5 × 0.0019) = .0231 AEB, which calculates to a theoretical concentration of 0.002 pg/ml

Dilution Linearity with CSF

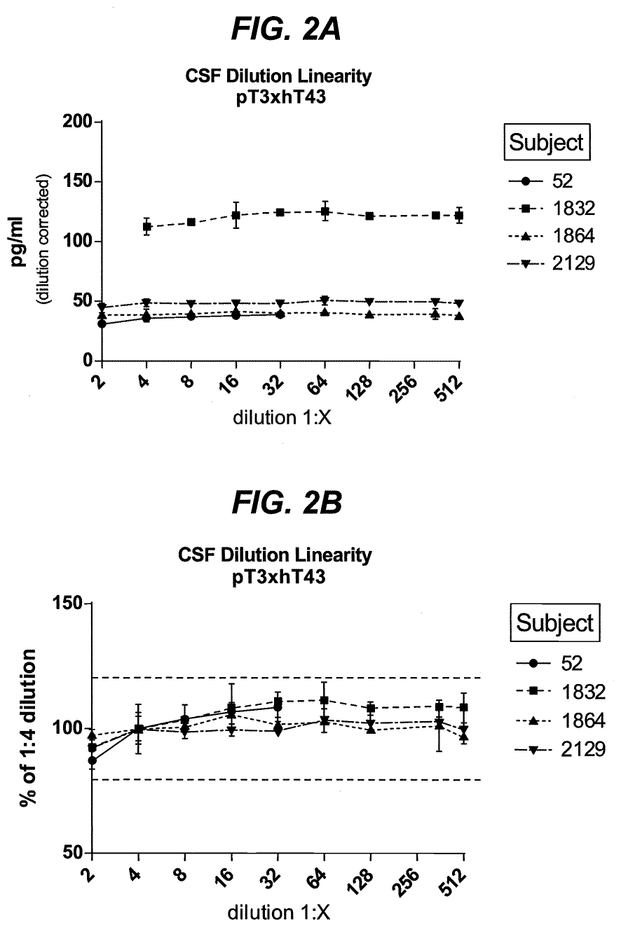

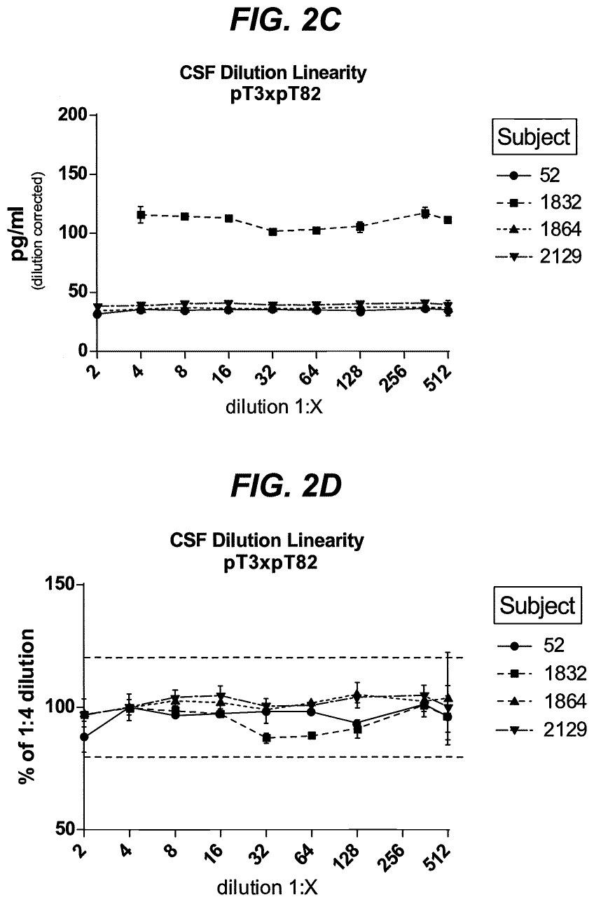

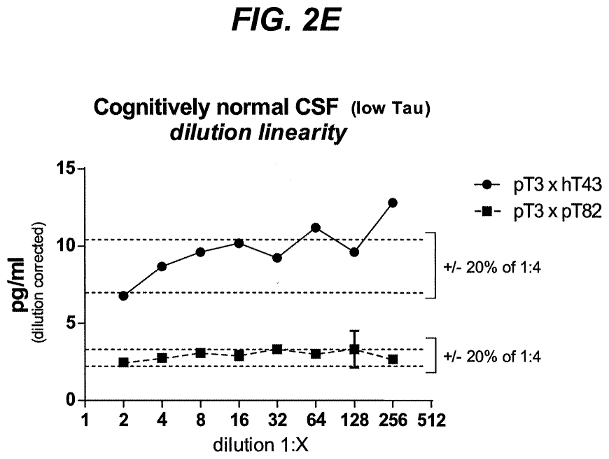

To assess dilution linearity and determine ideal dilution for testing CSF samples, a panel of 4 CSF samples from AD subjects (high tau, low AB42) was titrated from 1:2 to 1:4096 dilution in assay buffer and measured in the p217+ tau assays. Samples diluted beyond 1:512 typically measured below LLOQ. Sample measurements from 1:4 through 1:512 were dilution linear, so that was the defined range for measuring CSF samples. To confirm in Cognitively Normal subjects, a pool of CSF from subjects with low tau and high AB42 was measured similarly. Dilution linearity was again observed for 1:4 through 1:256 dilution, and beyond this range the measurements fell below LLOQ (FIG. 2).

### Precision

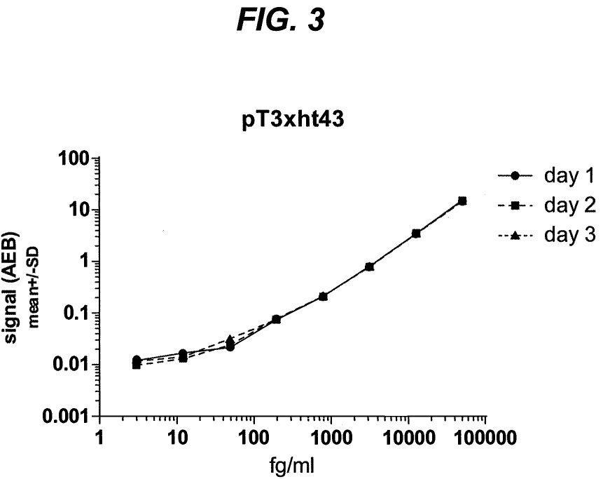

To assess precision of the measurements, the standard curve for pT3xhT43 was prepared and measured on 3 separate days (FIG. 3 and Table 3). Calibrant peptide was diluted from 30 to 0.041 pg/ml in serial 1:3 jumps and measured in duplicate on the pT3xhT43 assay. The procedure was repeated over 3 successive days at the same site and by the same technician. Analysis of the 4 points in middle of the curve (where CSF samples are measured) indicated that the precision within a run (intra-test CV %) was always <10% and averaged from 2.46-5.18% CV, and the inter-test precision averaged 6.46% CV. These are well within the accepted limits of 20% CV for a research use only (RUO) assay and are in part attributed to the automated nature of all the ELISA steps in the Simoa HD-1 Analyzer.

TABLE 3
Intra-and inter-test precision of the pT3xhT43 assay

| | | | | | | | | | | Sum | | |
|---|---|---|---|---|---|---|---|---|---|---|---|---|
| | | | | | | | | | | of | | |
| | Run1 | | | Run2 | | | Run 3 | | | runs | | |
| fg/ml | Mean | | CV | Mean | | CV | Mean | | CV | Mean | | CV |
| calibrant | AEB | SD | % | AEB | SD | % | AEB | SD | % | AEB | SD | % |
| 49 | .0228 | .0005 | 2.03 | .0248 | .0024 | 9.64 | .0319 | .0005 | 1.41 | .0265 | .0048 | 18.04 |
| 195 | .0762 | .0019 | 2.55 | .0777 | .0011 | 1.39 | .0785 | .0028 | 3.52 | .0775 | .0012 | 1.54 |
| 781 | .2130 | .0084 | 3.95 | .2161 | .0200 | 9.26 | .0210 | .0129 | 6.14 | .2130 | .0030 | 1.43 |
| 3125 | .8012 | .0105 | 1.31 | .8193 | .0036 | 0.44 | .7460 | .0289 | 3.88 | .7888 | .0382 | 4.84 |
| Mean | | | 2.46 | | | 5.18 | | | 3.74 | | | 6.46 |

### Transferability Between Labs

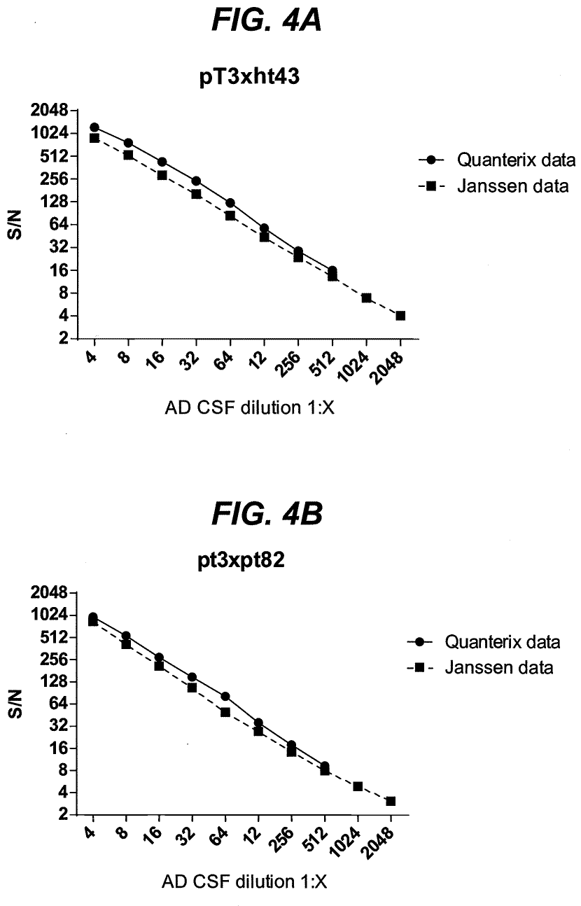

To evaluate precision of the p217+ tau assays between testing sites, the same AD CSF pool was measured, in titration, using the same lot of reagents at Janssen Neuroscience Biomarkers and at Quanterix Corporation. FIG. 4 shows that the measurements are very similar for the pT3xhT43 and pT3xpT82 assays at the two testing sites.

### Accuracy

To assess accuracy of the assays, two different pools of HV CSF were spiked with known concentrations of the calibrant peptides (0, 2, or 20 pg/ml), diluted to the recommended 1:4 dilution, and then measured in the pT3xhT43 and pT3xpT82 assays. This is one measure of potential interference presented by components of the sample matrix. Levels of endogenous signal were subtracted from the 2 and 20 pg/ml spike measurements, and then observed concentration of the calibrant material was compared to expected concentration to calculate percent recovery. The measured concentrations were compared to the expected concentrations to calculate spike recovery, yielding average recovery of 114% (Table 4). This is well within the accepted limits of 80-120% recovery for an RUO assay, indicating no significant interference in CSF when tested at ≥1:4 dilution.

TABLE 4
Spike recovery of the pT3xhT43 assay

| | Calibrant | | | | | Dilution | |
|---|---|---|---|---|---|---|---|
| CSF | spike | | Calculated | pg/ml | Average | corrected | % |
| sample | (pg/ml) | AEB | pg/ml | CV % | pg/ml | pg/ml | recovery |
| 1 | 0 | 1.4863 | 0.945 | 3 | 0.924 | 3.69 | NA |
| | | 1.4209 | 0.902 | | | | |
| | 2 | 2.2451 | 1.44 | 2 | 1.42 | 5.69 | 100 |
| | | 2.1776 | 1.40 | | | | |
| | 20 | 8.9800 | 6.33 | 2 | 6.40 | 25.6 | 110 |
| | | 9.1679 | 6.48 | | | | |
| 2 | 0 | 1.9960 | 1.28 | 6 | 1.23 | 4.91 | NA |
| | | 1.8397 | 1.18 | | | | |
| | 2 | 2.8076 | 1.82 | 2 | 1.85 | 7.39 | 124 |
| | | 2.8886 | 1.88 | | | | |
| | 20 | 10.1636 | 7.28 | 1 | 7.34 | 29.4 | 122 |
| | | 10.3026 | 7.40 | | | | |

| | |
|---|---|
| AVERAGE RECOVERY | 114% |

Competition of Signal by p217+ Directed Antibodies in CSF

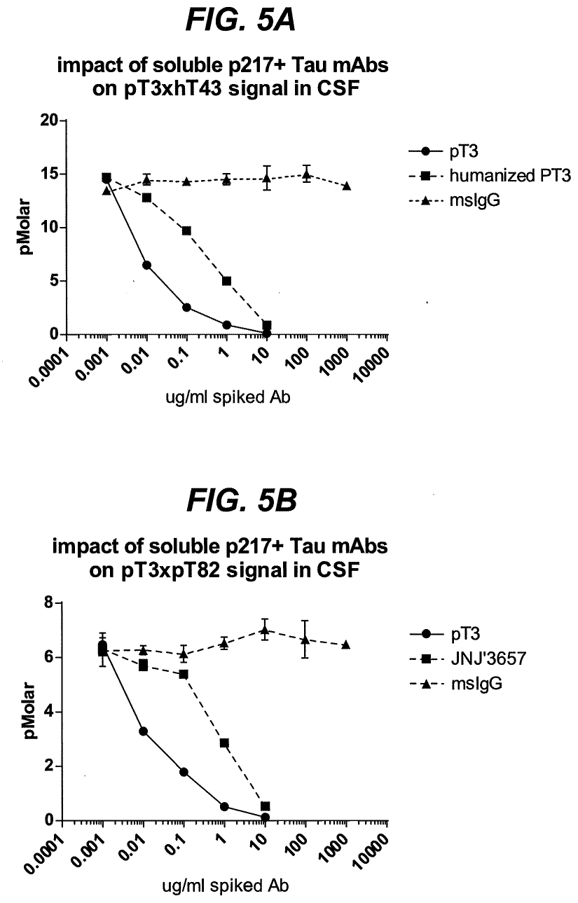

To confirm accuracy of the pT3xhT43 and pT3xpT82 assay signals in CSF, and to assess its potential utility as a pharmacodynamic assay in clinical studies of p217+ tau-directed antibodies, a pool of AD CSF was spiked with titrations of pT3 mAb or humanized pT3 mAb and measured in the pT3xhT43 and pT3xpT82 assays after a 2 hour incubation at room temperature (FIG. 5). Administration of the soluble pT3 and humanized pT3 antibodies reduced signal in the pT3-based assays in a dose dependent manner. Spiking msIgG (negative control) at comparable concentrations did not impact any of the measures. The lower competition ability of humanized pT3 mAb vs. pT3 can be attributed to the higher affinity of pT3 for p217+ tau.

### Phosphorylation Dependency

To confirm the signal in CSF obtained with the pT3xhT43 and pT3xpT82 assays was indeed based on a phosphorylated epitope, AD CSF was treated with alkaline phosphatase to dephosphorylate all residues. The samples were then analyzed in the pT3 assays and in two hT7-based assays, hT7xpT82 or hT7xBT2. hT7 is known to be not dependent on phosphorylation, so it was used as a negative control.

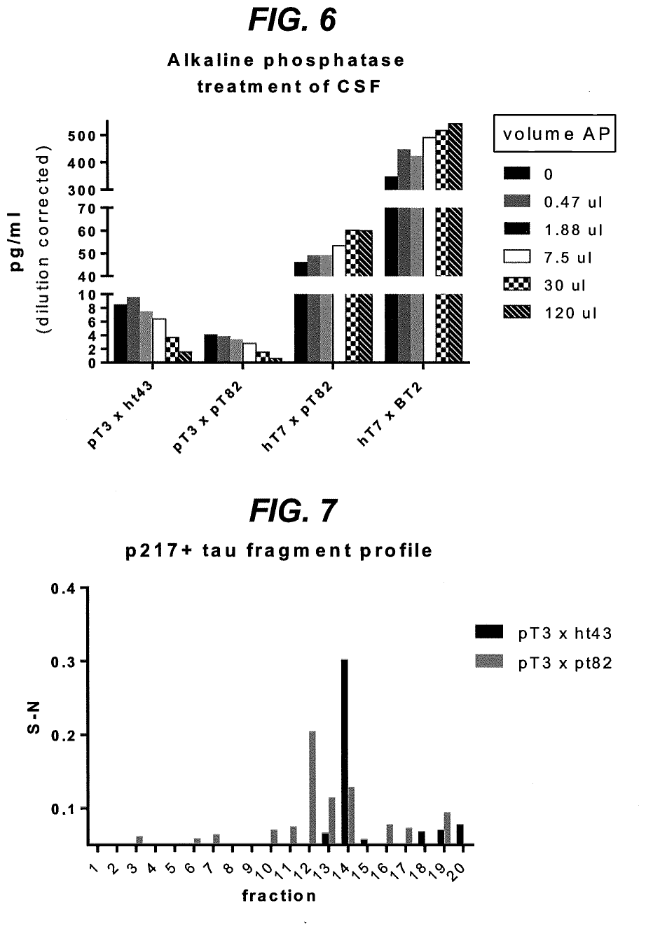

Pooled CSF from AD patients was treated with increasing amounts of alkaline phosphatase (AP) at 37° C. for 4 hrs in a zinc and magnesium chloride-containing buffer. The effect on the pT3-directed epitope was measured using the pT3xhT43 and pT3xpT82 assays. pT3xhT43 and pT3xpT82 signal was reduced by alkaline phosphatase treatment in a dose dependent manner. However, the non-phosphorylation dependent assays hT7xpT82 or hT7xBT2 did not show a signal decrease, they in fact showed an increase, as expected since pT7 binding is reduced by phosphorylation (FIG. 6).

p217+ Tau Fragment Profile

To explore the nature of the p217+ tau signal derived from measuring crude CSF, a sample of AD CSF was fractionated by rpHPLC via a method similar to that described in Meredith et al. PLoS One. 8(10):e76523, 2013. Fractions were collected and measured using the pT3xhT43 and pT3xpT82 assays (FIG. 7). In this chromatography format, smaller tau fractions elute sooner (smaller fraction number), while larger fractions elute later (larger fraction number). Full length tau elutes at fraction 19. The tau fragment profile indicated that there was very little full length tau detected by either of the assays, in line with prior reports (Meredith et al. PLoS One. 8(10):e76523, 2013, Barthelemy et al., J Alzheimers Dis. 51(4):1033-43, 2016). The pT3xpT82 assay detected two major peaks (tau species) that were smaller than full length tau (fractions 12 and 14), while pT3xhT43 assay detected only one of these major peaks (fraction 14). This indicated that p217+ tau in CSF exists in at least two fragments, a larger fragment encoding at least the region from hT43 to pT3 (aa 7-220 of tau) and a smaller fragment encoding at least the region from pT82 to pT3 (aa 116-220 of tau) but not reaching all the way to the hT43 epitope. That is, there is likely a proteolytic cleavage site between aa 20 and aa 116 that is only cleaved in a subset of tau molecules at any given time. The profile is not p217+ specific, as measurements with other tau assays that recognize a similar region of tau but are not phosphorylation-specific yield similar findings (data not shown).

### Analyte Stability

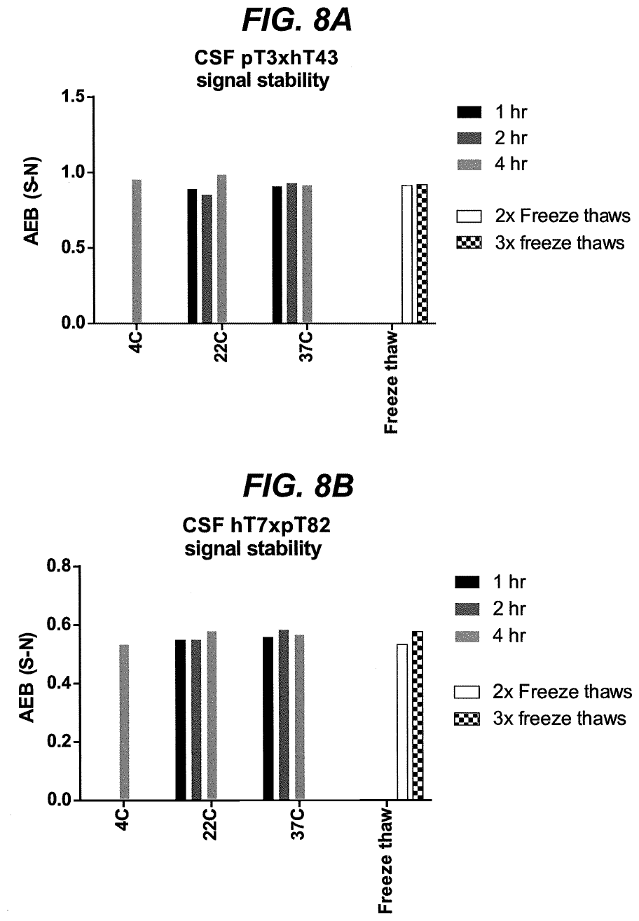

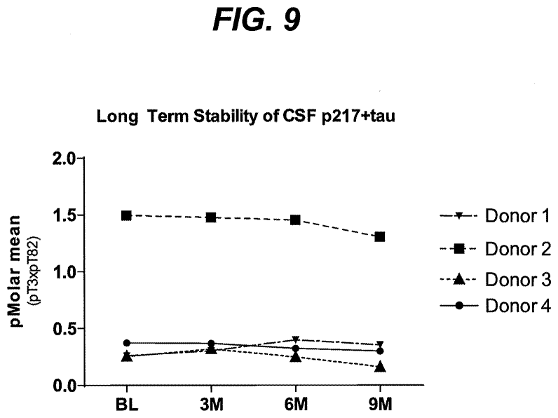

The stability of the endogenous p217+ tau epitope was assessed at various temperatures. A pool of AD CSF was aliquoted, and each aliquot subjected to storage at 4° C., 22° C., or 37° C. for 1, 2, or 4 hrs. Also, a subset of aliquots were freeze-thawed (−80° C. to 22° C.) 2 or 3 times. All samples were then diluted 1:20 and analyzed using pT3xhT43 and hT7xpT82 assays (FIG. 8). No significant change in signal was observed in any of the conditions tested, indicating that all 4 epitopes recognized by these assays are sufficiently stable to enable standard storage/testing procedures. Finally, CSF was collected prospectively from 4 donors then aliquoted and frozen at −70 C, samples were removed every 3 months for measurement with pT3xpT82 assay. No significant change in signal was observed at 3, 6, or 9 month timepoints (FIG. 9).

### Example 8. Clinical Qualification of pT3xhT43 and pT3xpT82 Assays

To assess the utility of the pT3xhT43 and pT3xpT82 assays in diagnosis and staging of AD, three cohorts of CSF samples were obtained for p217+ tau measurement. Measurements were analyzed for correlation with cognition scores and with other classical AD biomarkers.

### Cohort 1: “Interassay Correlation Cohort”

CSF samples, VF and LF, and brain biopsy (ventricle) were obtained from 10 subjects with the neurodegenerative disorder Normal Pressure Hydrocephalus (NPH), a condition characterized by excessive interstitial fluid production in the brain and presenting at a high incidence with AD. p217+ tau measurements were performed on crude CSF and analyzed for correlation with the traditional AD biomarkers.

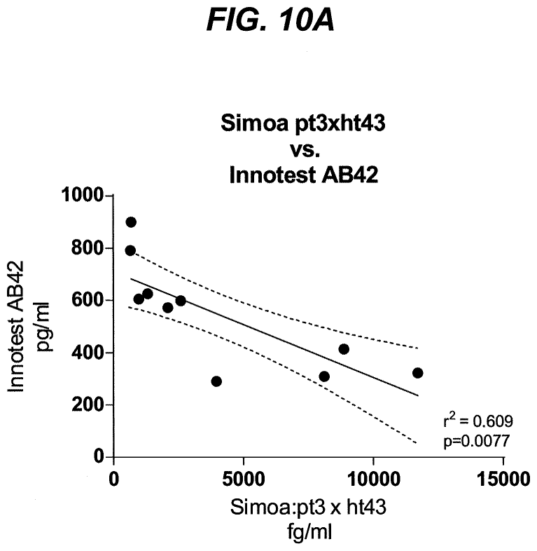

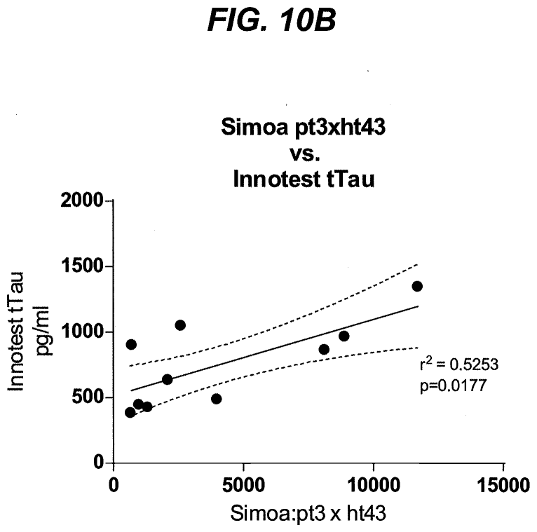

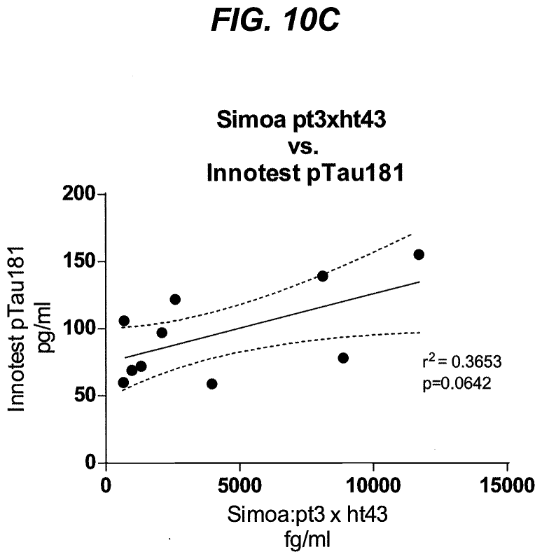

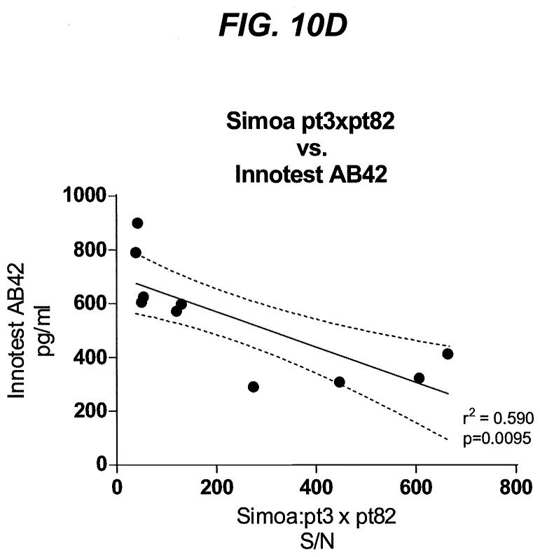

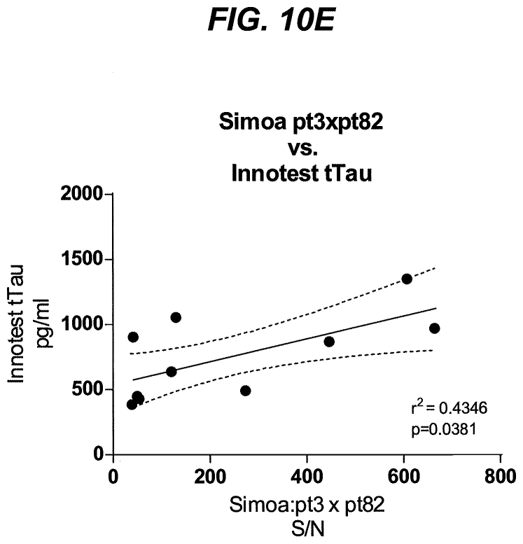

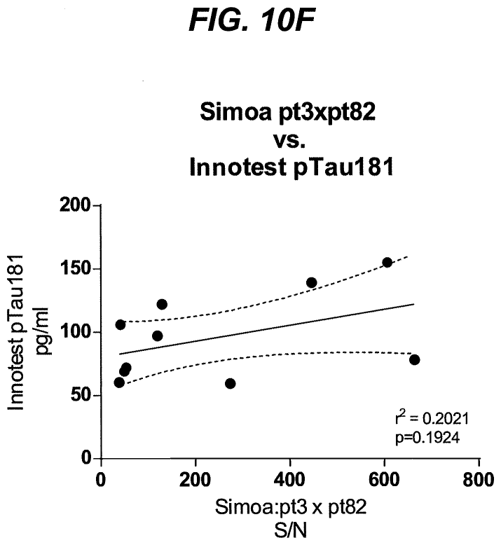

Levels of Aβ42 (FIGS. 10A, 10D), tTau (FIGS. 10B, 10E), pTau181 (FIGS. 10C, 10F) in VF were determined by Innotest ELISA (classical measurement). The same samples were measured with the pT3xhT43 (FIGS. 10A, 10B, 10C) and pT3xpT82 (FIGS. 10D, 10E, 10F) assays, and correlations were evaluated. Both pT3xhT43 and pT3xpT82 assays revealed a negative correlation with CSF Aβ42 (r²=0.609, p=0.0077 and r²=0.590, p=0.0095, respectively), and a positive correlation with CSF tTau (r²=0.525, p=0.0177 and r²=0.435, p=0.0381, respectively), but they did not significantly correlate with CSF pTau181 (FIG. 10).

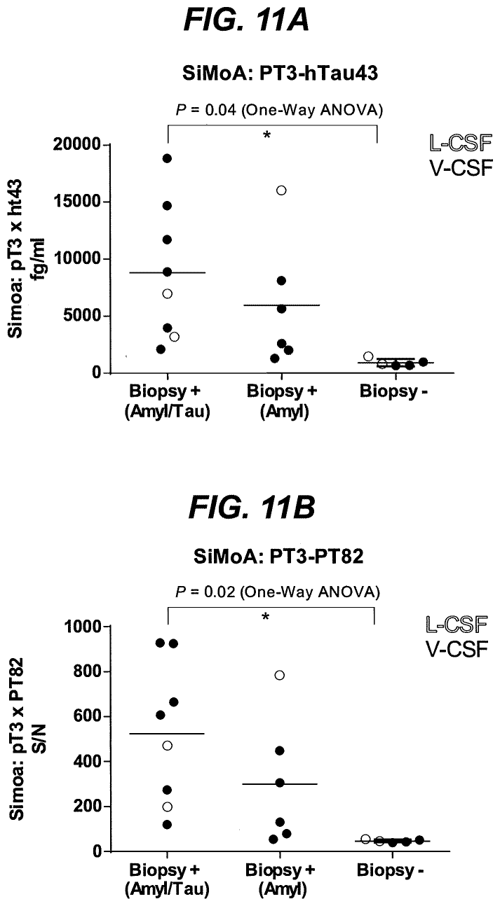

Brain biopsy from the same 10 NPH subjects was analyzed by IHC and pathologist-scored as amyloid positive/negative and tau positive/negative. When positive for both, the sample was designated “Biopsy +” and was a classical diagnosis for AD. When negative for both, the sample was designated “Biopsy −” and was a classical diagnosis for non-AD. Samples designated “Biopsy+(Amyl)” were positive for amyloid but negative for tau. CSF obtained from ventricular tap (VF=black dots) or lumbar tap (LF=red dots) was measured with the pT3xhT43 and pT3xpT82 assays, and correlations were evaluated (FIG. 11). Both pT3xhT43 and pT3xpT82 assays were able to separate brain biopsy negative (amyloid−/tau−) from positive samples (amyloid+/tau+) (p=0.04 and 0.02, respectively). Samples positive for amyloid, but not for tau, often measured between the biopsy+ and biopsy− samples. Amyloid plaques in the brain are believed to precede tau tangles, therefore amyloid+/tau− samples may represent early AD or another disease.

Cohort 2: “HV vs. AD Cohort”

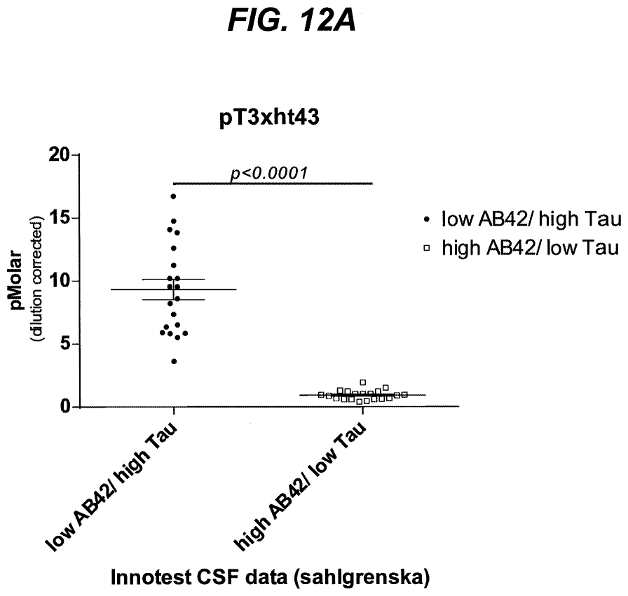

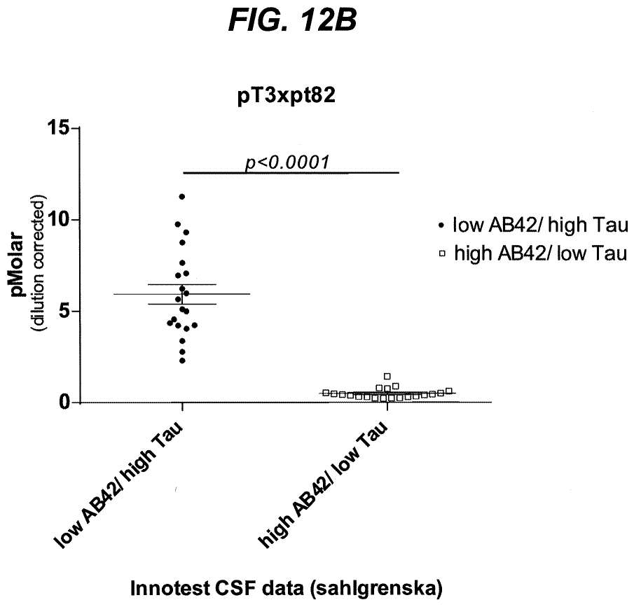

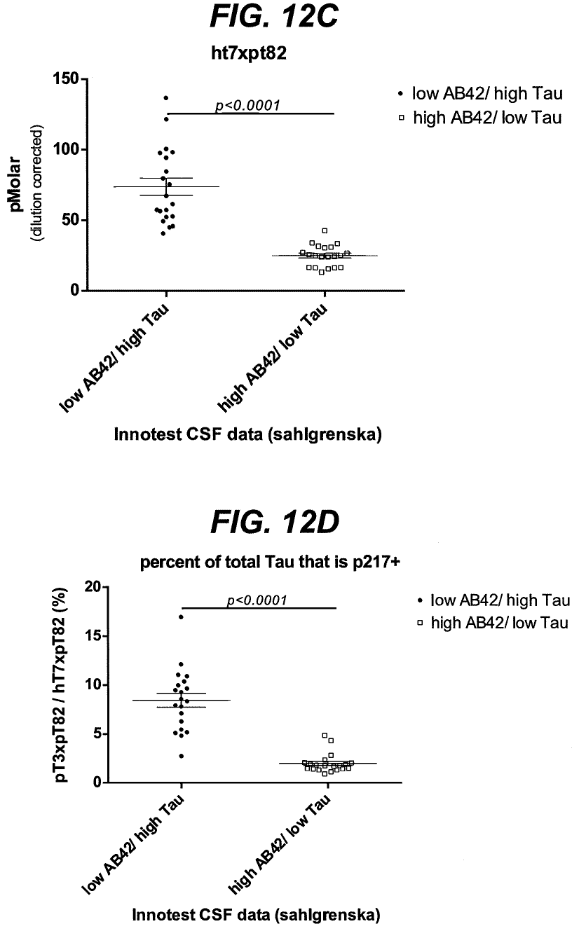

CSF samples (LF) from biochemically-defined AD vs. HV subjects (n=20 per group) were obtained from University of Sahlgrenska. Levels of Aβ42 and tTau were determined by Innotest ELISA (classical measurement) to subdivide the groups (AD=CSF Aβ42<400 pg/ml AND CSF tTau>600 pg/ml, HV=CSF Aβ42>400 pg/ml AND CSF tTau<600 pg/ml). Measurements were performed using pT3xhT43, pT3xpT82 and hT7xpT82 assays on crude CSF and a subset of rpHPLC-fractionated CSF. The results were analyzed for correlation with the traditional AD biomarkers (FIG. 12). The data in panels A and B of FIG. 12 demonstrated that the pT3 epitope is an indicator of patients at high risk of rapidly progressing to insipient AD. The pT3 epitope was highly elevated in patients demonstrating high total tau and low Aβ42. Conversely, the pT3 epitope was present at low levels in subjects with low total tau and high Aβ42. FIG. 11C confirmed that the elevated pT3 epitope-containing tau was driven at least in part by elevated levels of total tau, as demonstrated by the hT7xpT82 total tau assay, but not entirely (FIG. 12D). This indicates that both the amount of tau, and the extent to which it is phosphorylated at p217+ epitope, is elevated in AD.

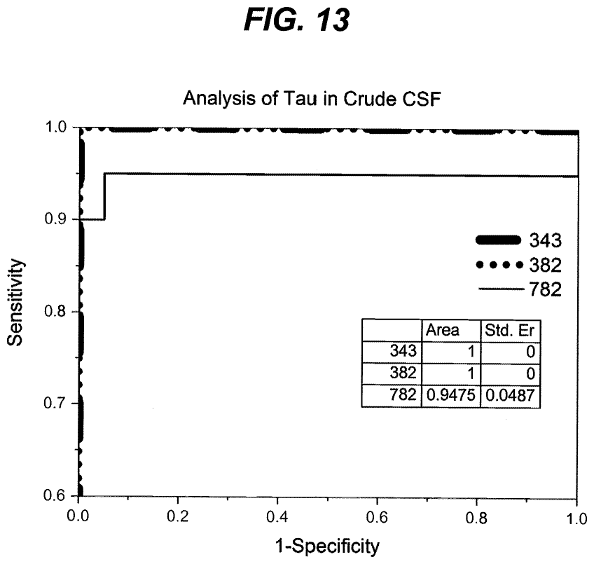

The data from FIG. 11 was used to create ROC curves for the ability of the pT3xhT43, pT3xpT82, and hT7xpT82 assays to differentiate the AD from HV samples. All three assays showed excellent specificity and sensitivity. However, the two pT3-based assays (pT3xhT43, pT3xpT82, which detect p217+ tau) had improved diagnostic power over the hT7-based assay (FIG. 13).

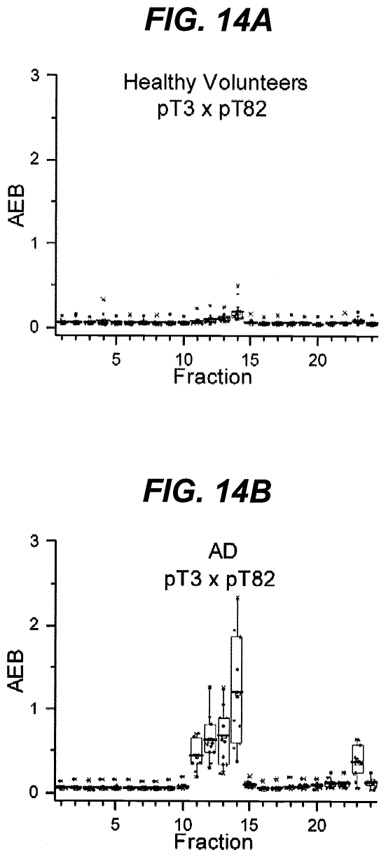

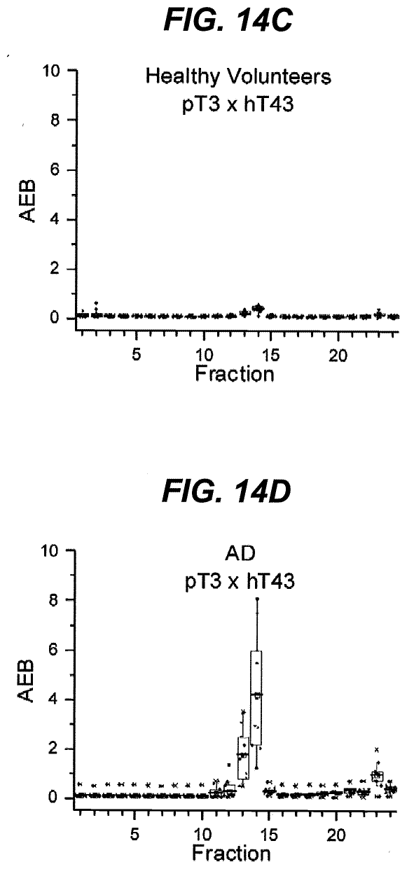

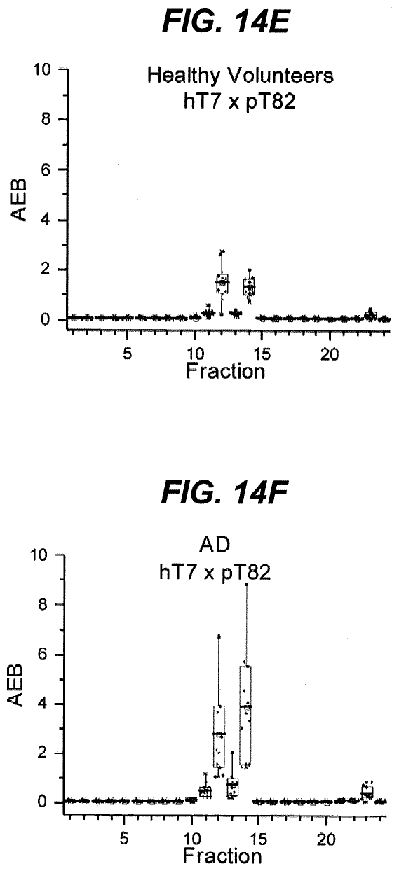

A subset of the same CSF samples measured in FIG. 12 (n=11 per group) were fractionated by rpHPLC and then measured with pT3xhT43, pT3xpT82, and hT7xpT82 assays (the latter was measure of the same tau fragments in a phosphorylation-independent manner) (FIG. 14). The profile of tau fragments observed was similar to that seen in Example 7 and FIG. 7. That is, two major species were seen with both pT82-based assays, pT3xpT82 and hT7xpT82, while only one of the peaks was seen with the pT3xhT43 assay. Both major species were present at higher concentrations in the AD group than the HV group. Further, the pT3-based assays (p217+ tau) showed a greater differential between the groups than the hT7-based assay (total tau), as detected in the crude CSF analysis. The larger p217+ tau species (Fractions 13-14) provided the largest AD vs. HV differential (FIG. 14).

The sum of all major tau fragments in FIG. 14 (fractions 11-14) was calculated, then compared between the AD and HV subgroups. The percent increase of pT3-epitope-containing tau (pT3xhT43 or pT3xpT82) in AD was greater than twice that seen with non-pT3-epitope containing tau (hT7xpT82) (Table 5).

TABLE 5
Total tau signal in pT3 vs. non-pT3 assays

| | Mean | Standard | Mean | Standard | % |
|---|---|---|---|---|---|
| | AEB in | Deviation | AEB in | Deviation | Increase |
| Assay | AD | in AD | HV | in HV | in AD |
| pT3/pT82 | 0.74 | 0.32 | 0.12 | 0.05 | 620 |
| pT3/hT43 | 1.65 | 1.88 | 0.22 | 0.14 | 750 |
| hT7/pT82 | 2.00 | 1.66 | 0.84 | 0.66 | 250 |

Analysis of the signal in each FIG. 14 fraction independently, vs. as a sum as done in Table 5, was performed to reveal which fraction yielded the greatest AD vs. HV signal. The most informative fragment pool was detected using the pT3 antibody on fragment pools 13 and 14 (Table 6).

TABLE 6
Tau signal in different tau fragment pools in pT3 vs. non-pT3 assays
pT3 Assay Identifies AD patients better than a
Non-pT3 Assay: Differences per Fragment
Pool

| | | Mean | Mean | % AD |
|---|---|---|---|---|
| | Fragment | AEB | AEB | over |
| Assay | Pool | in AD | in HV | HV |
| pT3xhT43 | 11 | 0.22 | 0.105 | 210 |
| | 12 | 0.333 | 0.113 | 295 |
| | 13 | 1.78 | 0.235 | 757 |
| | 14 | 4.25 | 0.407 | 1044 |
| pT3xpT82 | 11 | 0.447 | 0.079 | 566 |
| | 12 | 0.631 | 0.098 | 644 |
| | 13 | 0.681 | 0.121 | 563 |
| | 14 | 1.2 | 0.193 | 622 |
| hT7xpT82 | 11 | 0.51 | 0.29 | 176 |
| | 12 | 2.8 | 1.51 | 185 |
| | 13 | 0.73 | 0.26 | 281 |
| | 14 | 3.94 | 1.31 | 301 |

Cohort 3: “HV Vs. ARAD Vs. Early Stage AD Cohort”

CSF samples (LF) from clinically defined normal (CDR 0) vs. mild memory complaint (CDR 0.5) subjects (n=20 per group) were obtained from Janssen study ALZ1005/2002. Levels of Aβ42, tTau, and pTau181 were determined by Innotest ELISA. Based on the CDR and CSF Aβ42 scores, the subjects were classified into (a) HV=CDR 0 and Aβ42>600 pg/ml, (b) ARAD=CDR 0 and Aβ42<600 pg/ml, (c) potentially non-AD dementia=CDR 0.5 and Aβ42>600 pg/ml, and (d) early stage AD=CDR 0.5 and Aβ42<600 pg/ml.

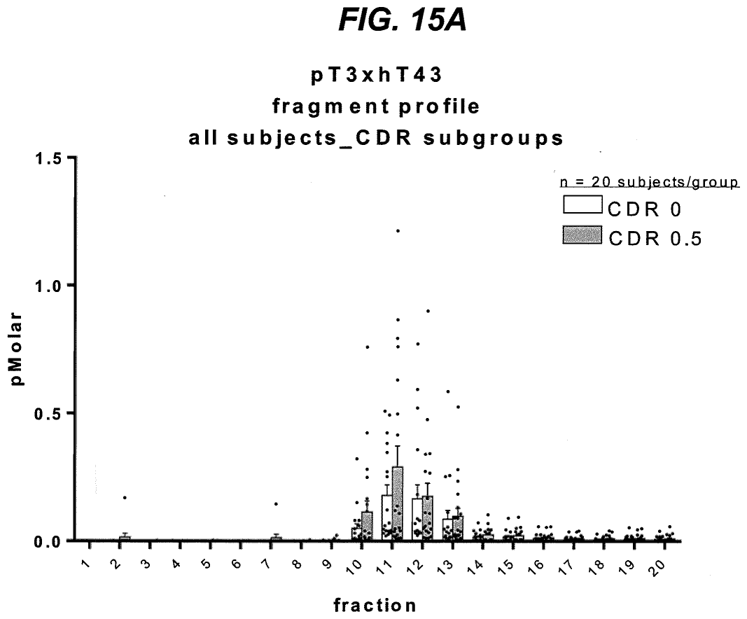

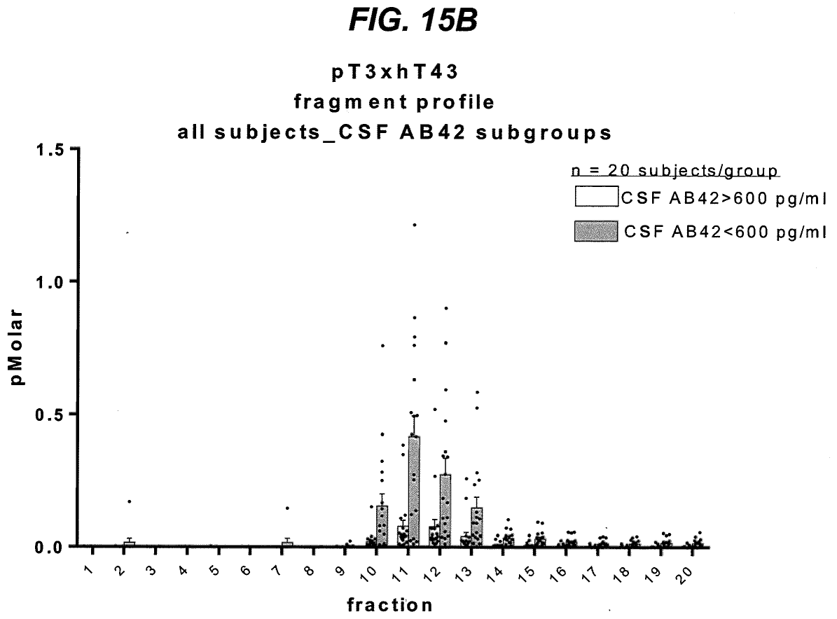

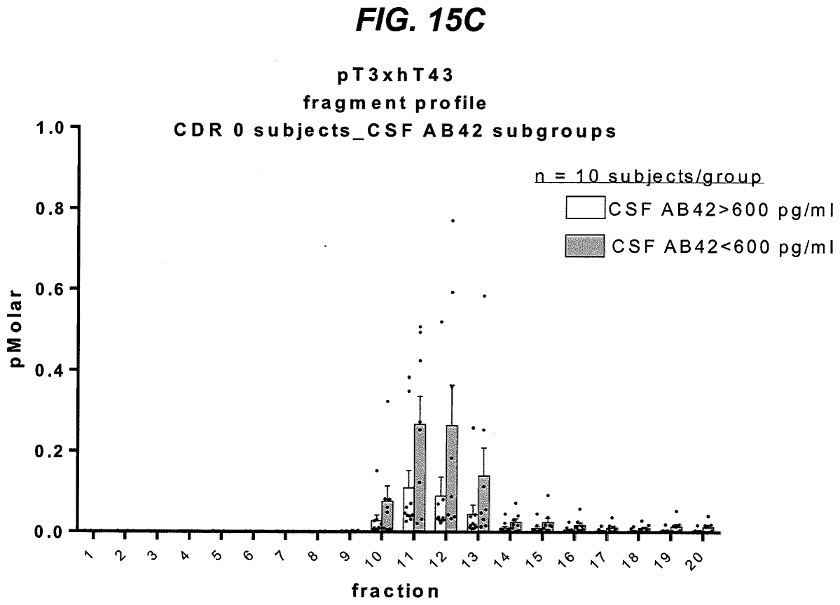

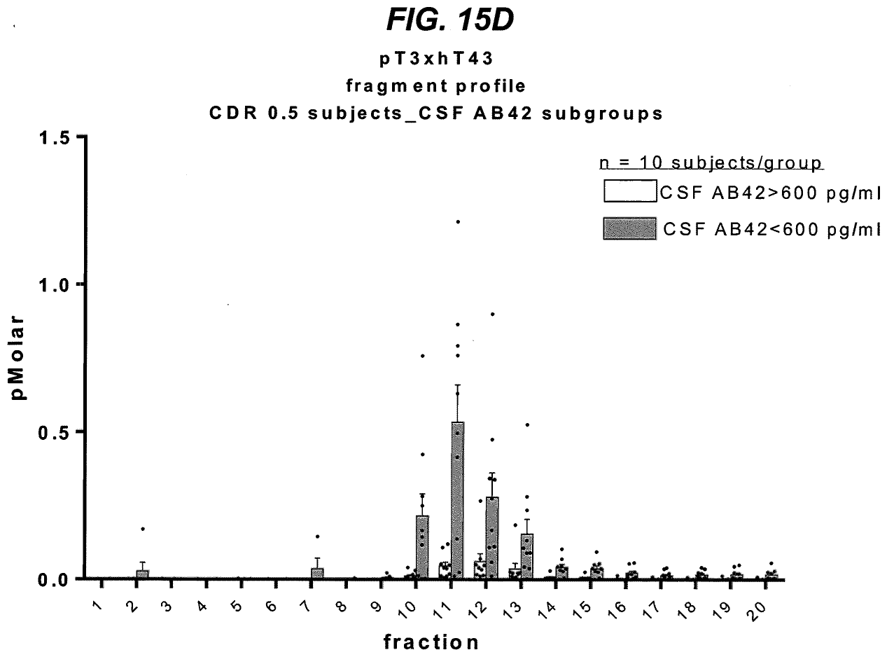

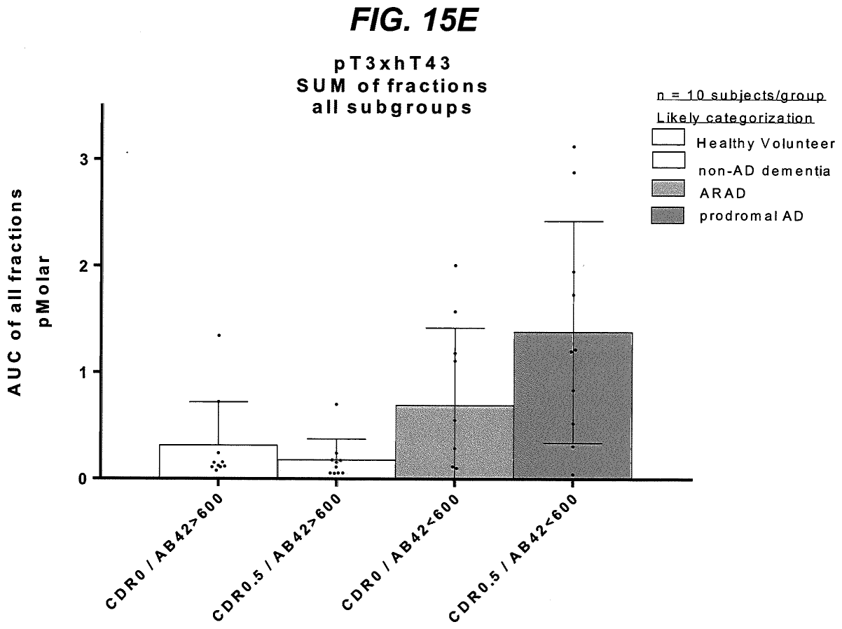

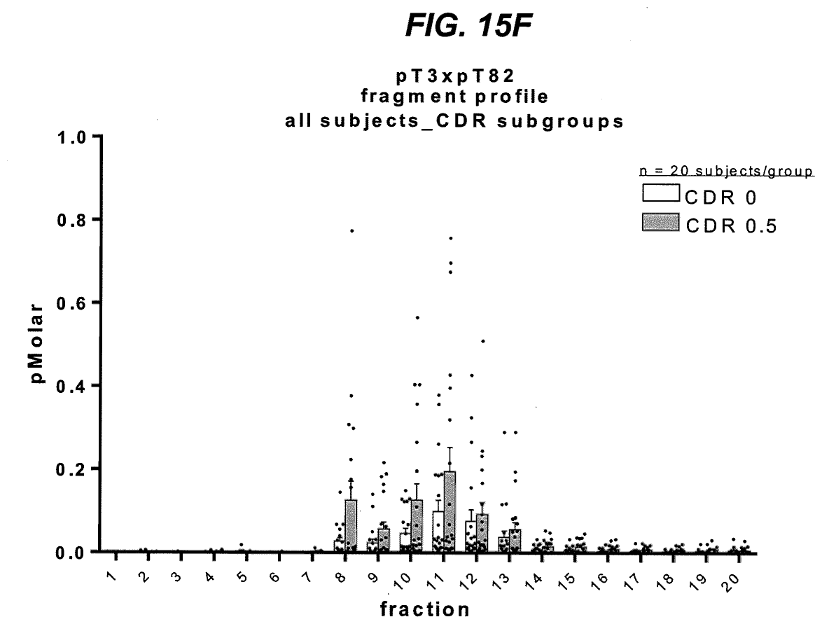

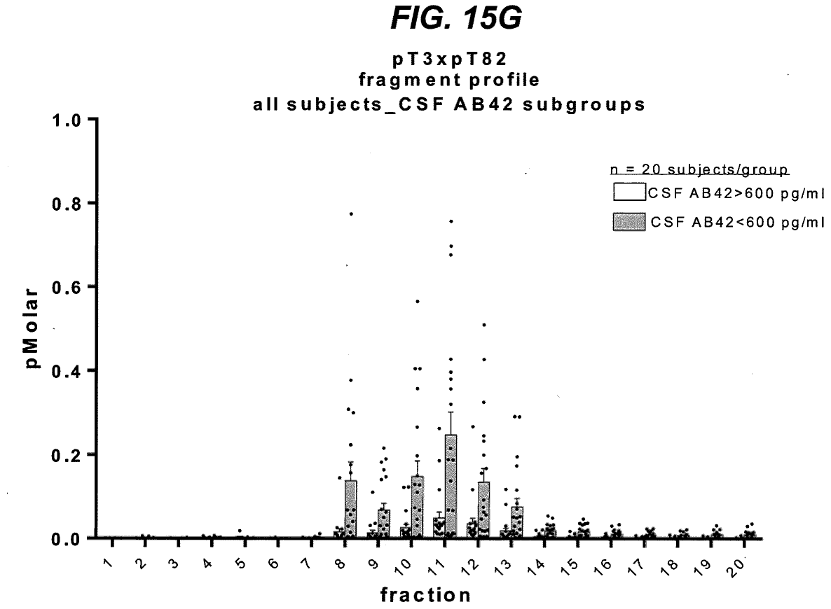

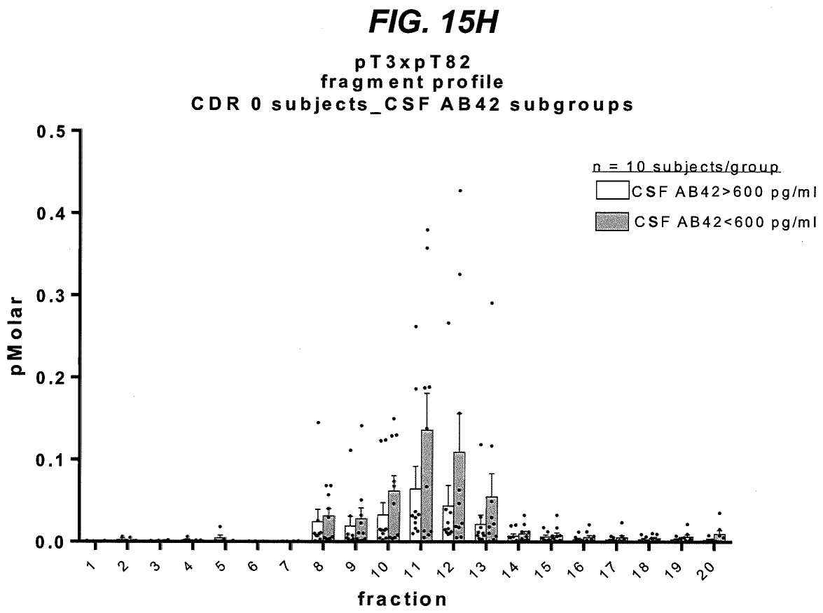

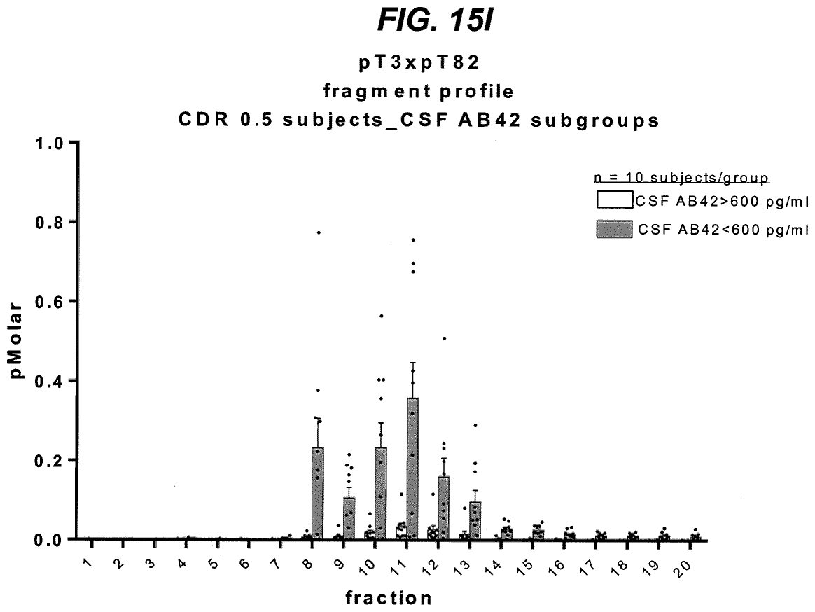

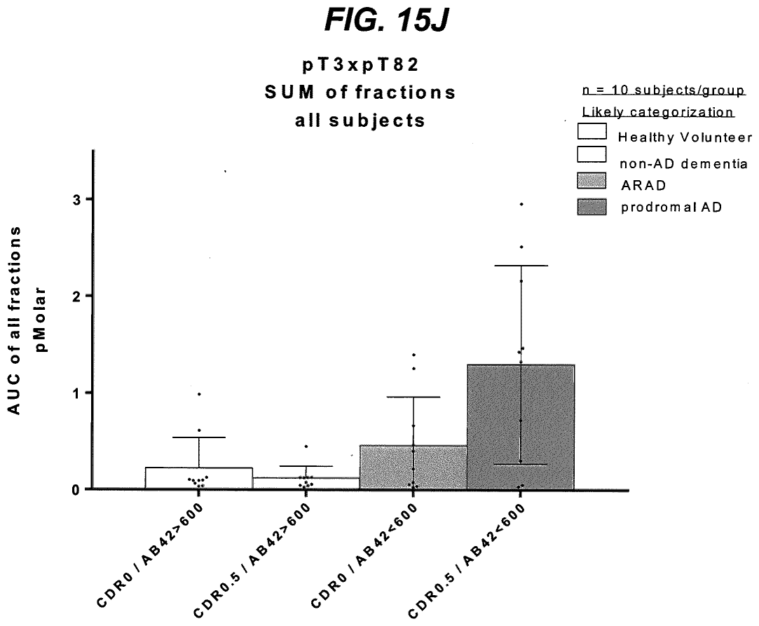

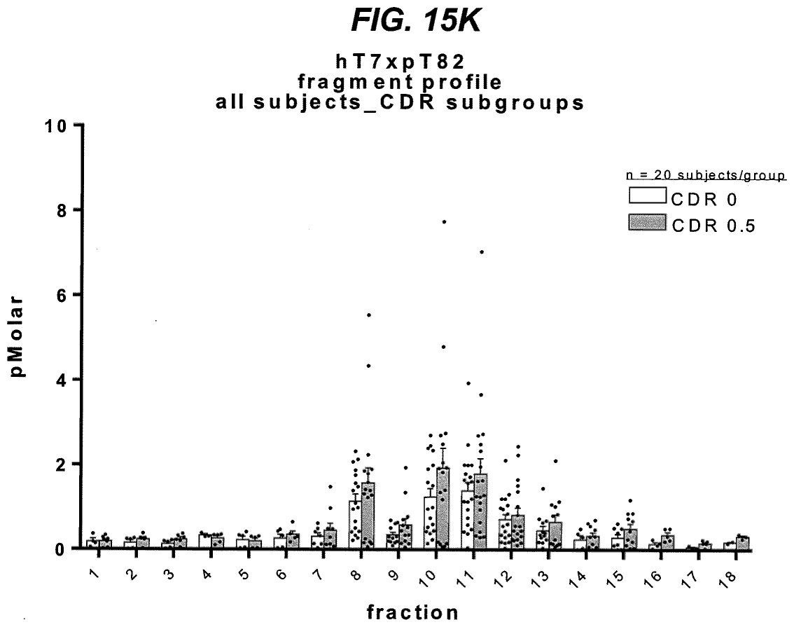

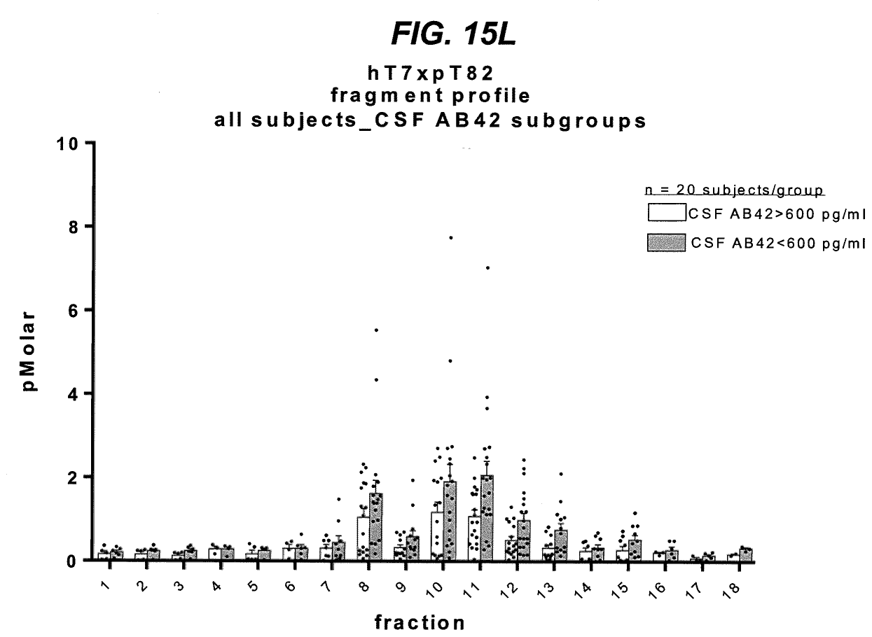

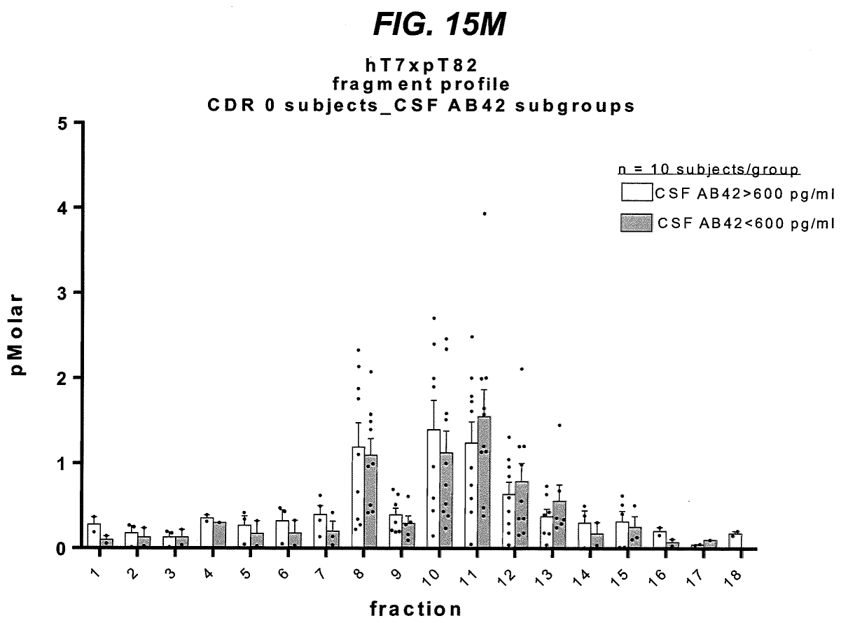

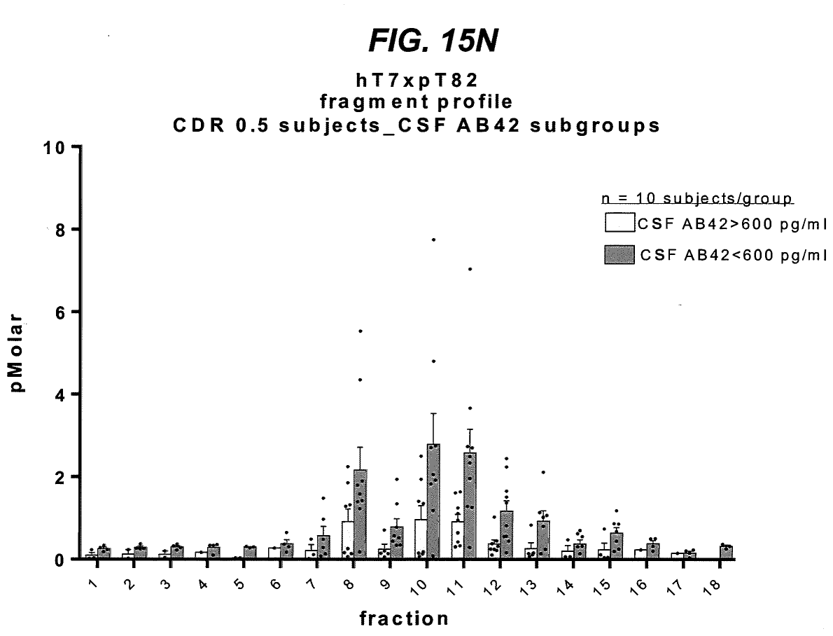

CSF samples were also fractionated by rpHPLC and measured with pT3-based (pT3xhT43, FIGS. 15A-15E and pT3xpT82, FIGS. 15F-15J) and total tau (hT7xpT82, FIGS. 15K-15O) assays. All the pT3-based and hT7-based assays showed elevated signal in CDR 0 vs. 0.5 (FIGS. 15A, 15F and 15K), and in samples with Aβ42<600 pg/ml vs. >600 pg/ml (FIGS. 15B, 15G and 15L). Breakdown by CDR×Aβ42 level is shown in FIGS. 15C, 15D, 15H, 15I, 15M and 15N, and summed signal across all fractions is illustrated in FIGS. 15E, 15J and 15O. The signal levels were highest in the Aβ42<600 pg/ml+CDR 0.5 subgroup, consistent with elevated p217+ tau signal in early stage AD vs. HV or ARAD. The separation between subgroups was superior in pT3-based assays vs. hT7-based assay, indicating that hyper-phosphorylation of the pT3 epitope is particularly enriched (above simple total tau elevation) in disease.

### Cohort 4 (“CDR 0 vs CDR1 Cohort”)

LF CSF samples from clinically defined normal (Clinical Dementia Rating 0; CDR 0) vs. mild memory complaint (CDR 1) subjects (n=5 per group) were obtained from Washington University. CDR & MMSE, as well as CSF Aβ42, tTau, and pTau181 measurements by Innotest assays were obtained at Washington University. Prior to shipment, samples were coded so that Janssen was blind to sample identity or characterization. rpHPLC and Simoa tTau & p217+ tau measurements were performed at Janssen Neuroscience Biomarkers, La Jolla and sent to Washington University for analysis.

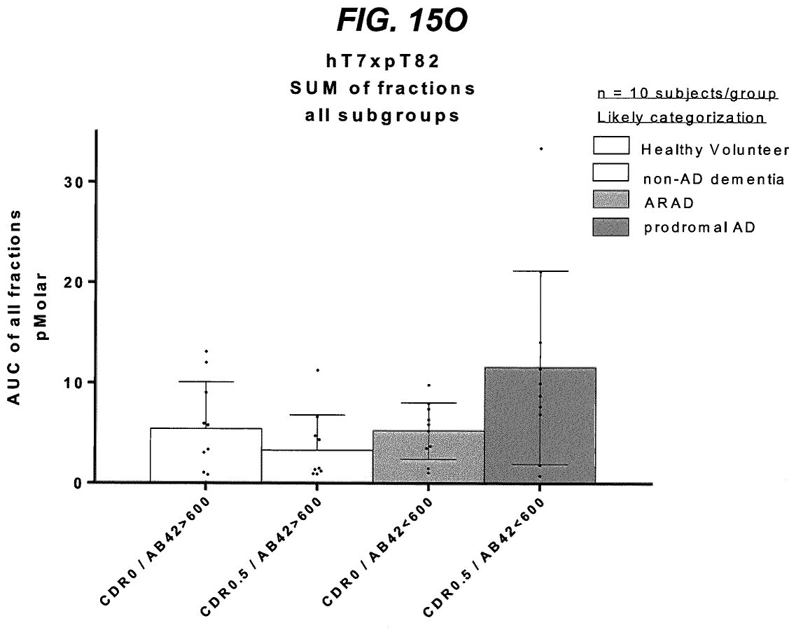

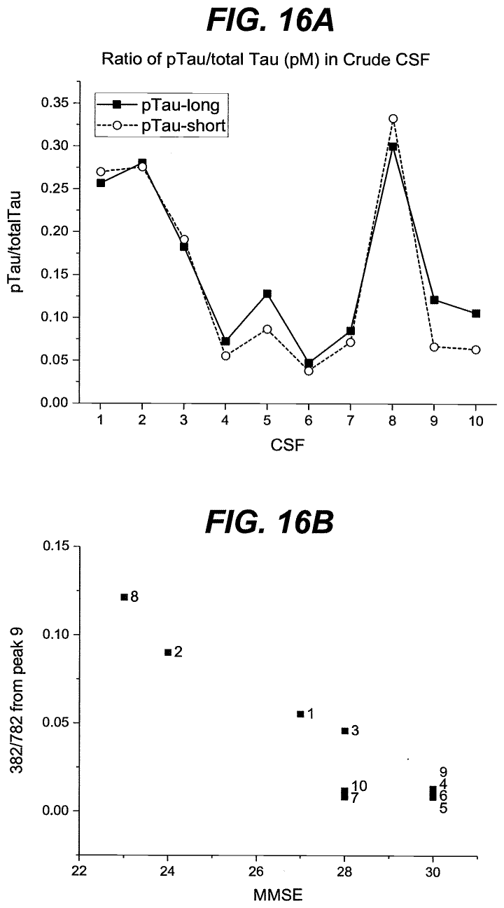

CSF samples were measured crude or after rpHPLC fractionation, using both pT3-based assays (pT3xhT43 & pT3xpT82) and tTau (hT7xpT82). The data was expressed as a ratio between the two pT3 assays (Table 7), to assess relative impact of the short tau species, or as (FIGS. 16A-16B) a ratio between either pT3 assay and tTau, to assess relative impact of this phosphorylation event. In both cases the result accurately predicted CDR status for 9 of the 10 subjects. The 1 outlier subject was determined to also have abnormally low Tau by Innotest, and so may represent dementia from a non-tauopathy. Intriguingly a correlation between p217+ Tau/tTau ratio and MMSE was observed, suggesting the signal detected by the pT3-assays may track with cognition.

TABLE 7
Ratio of pT3xpT82 (p217 + short) vs pT3xhT43 (p217 + long) analysis on crude CSF

| | | | | | | | | P217 + tau |
|---|---|---|---|---|---|---|---|---|
| | | | | | Innotest Aβ, tTau, pTau | | | P217 + short/ |
| ID | CDR | MMSE | Gender | age_at_LP | Innotest Aβ | Inno_Tau | Inno-pTau | P217 + long |
| 24064* | 1 | 28 | M | 77 | + | 119.44 | 30.154 | low |
| 24593 | 1 | 23 | F | 55 | + | 816.427 | 104.47 | 1.25 |
| 25711 | 1 | 28 | M | 79 | + | 450.005 | 65.486 | 1.78 |
| 62496 | 1 | 24 | M | 85 | + | 1126.919 | 153.024 | 1.34 |
| 64397 | 0 | 30 | F | 72 | − | 261.846 | 44.805 | 0.81 |
| 64722 | 0 | 30 | F | 77 | − | 247.99 | 55.951 | 0.99 |
| 64996 | 0 | 30 | M | 80 | − | 427.871 | 96.301 | 0.82 |
| 65839 | 0 | 30 | F | 68 | − | 180.452 | 39.263 | 0.99 |
| 65922 | 0 | 28 | M | 58 | − | 539.75 | 96.29 | low |
| 68031 | 1 | 27 | M | 68 | + | 1080.048 | 120.47 | 1.22 |

*CDR 1 & Aβ positive but has low Tau & pTau on Innotest and Simoa

Cohort 5 (“HV Vs. MCI Vs. AD Cohort”)

LF CSF samples from clinically and biochemically (Innotest AB42>600 pg/ml) defined HV (n=7) were obtained from Precision Medicine (San Diego, CA). LF CSF samples from clinically and biochemically (Innotest AB42<600 pg/ml) defined MCI (n=28) and AD (n=12) were obtained from University of Antwerp. rpHPLC and Simoa p217+ tau measurements were performed at Janssen Neuroscience Biomarkers, La Jolla.

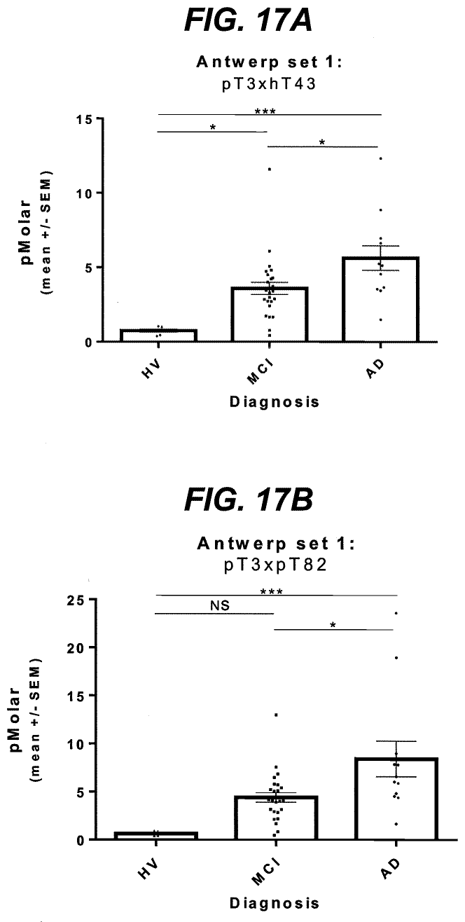

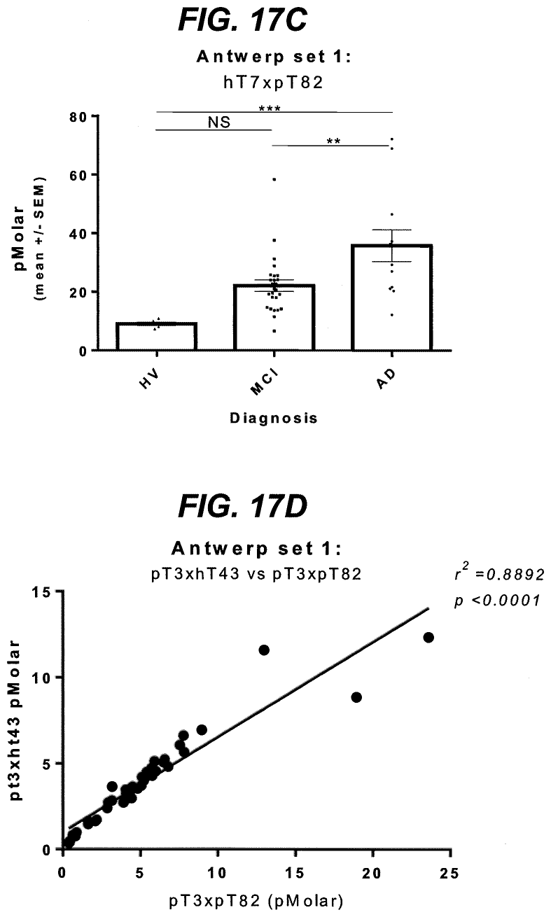

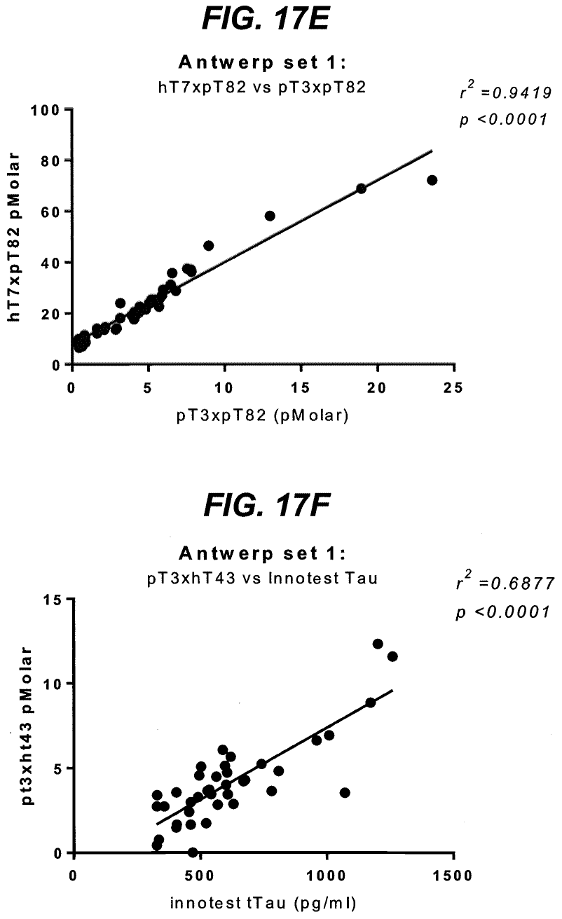

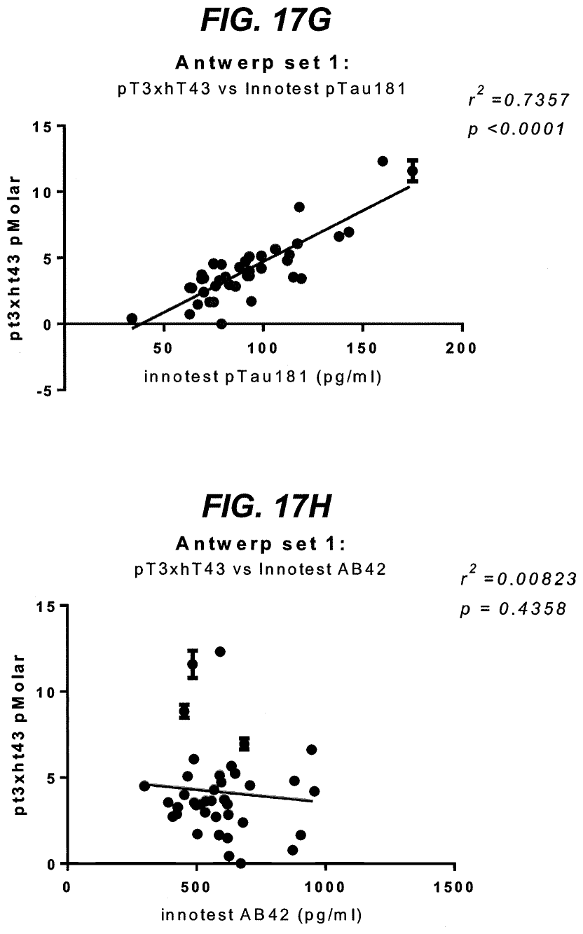

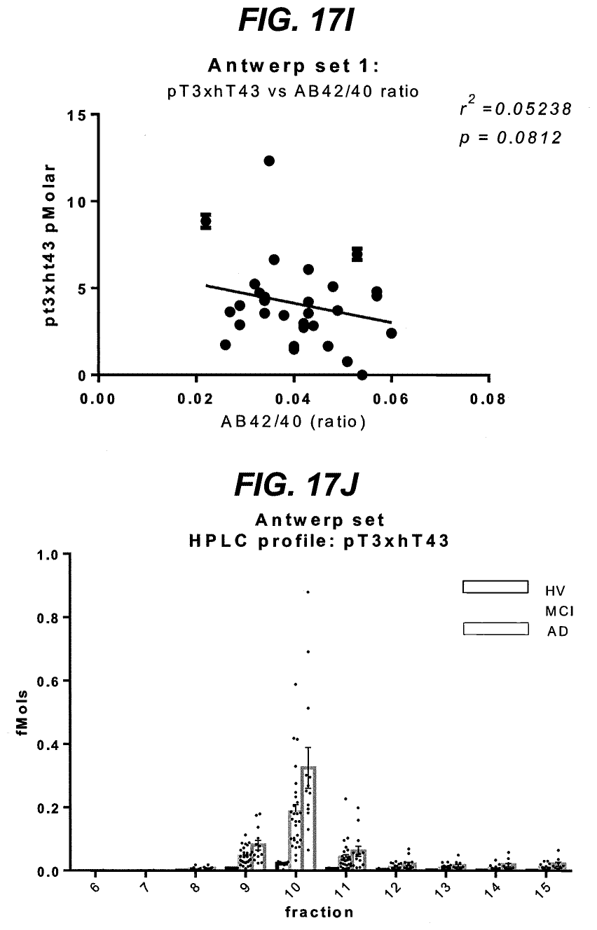

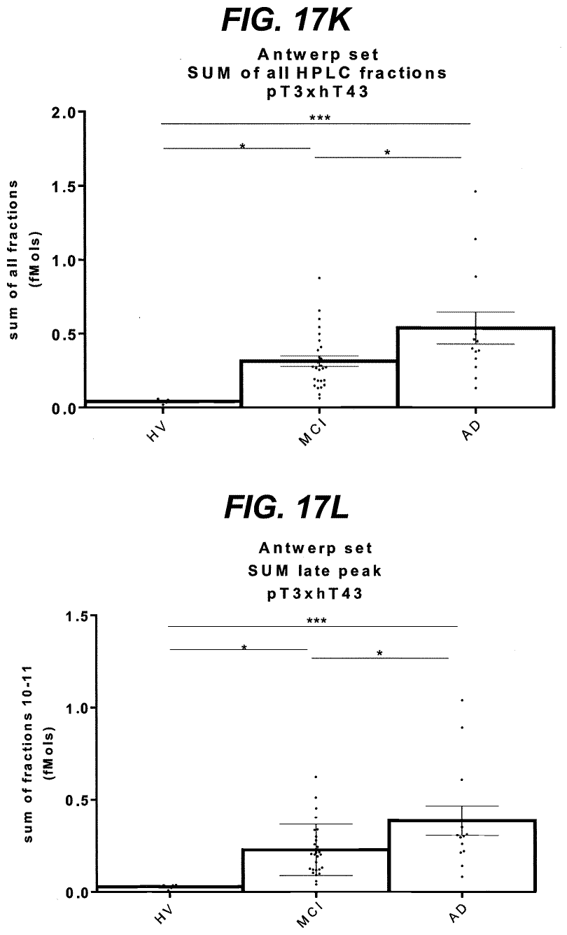

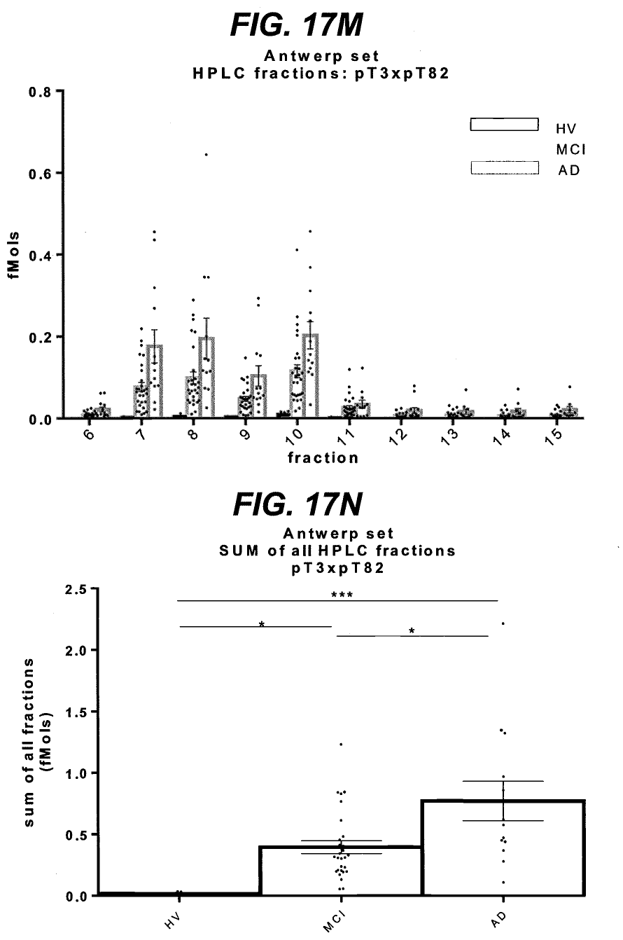

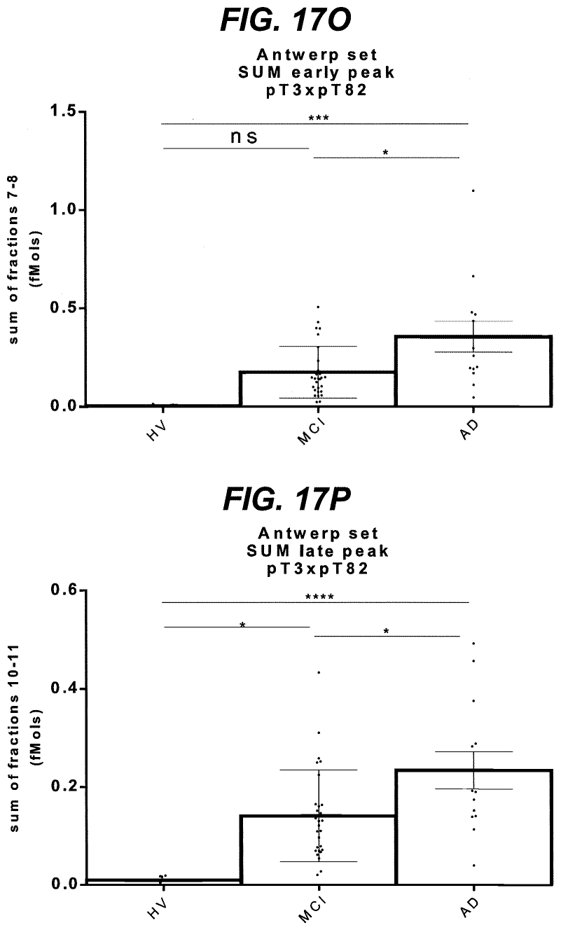

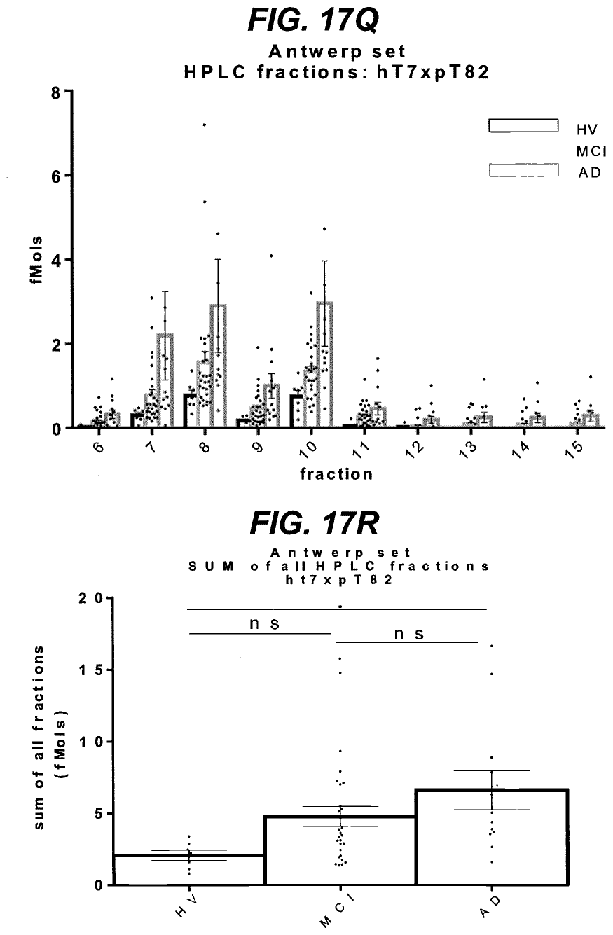

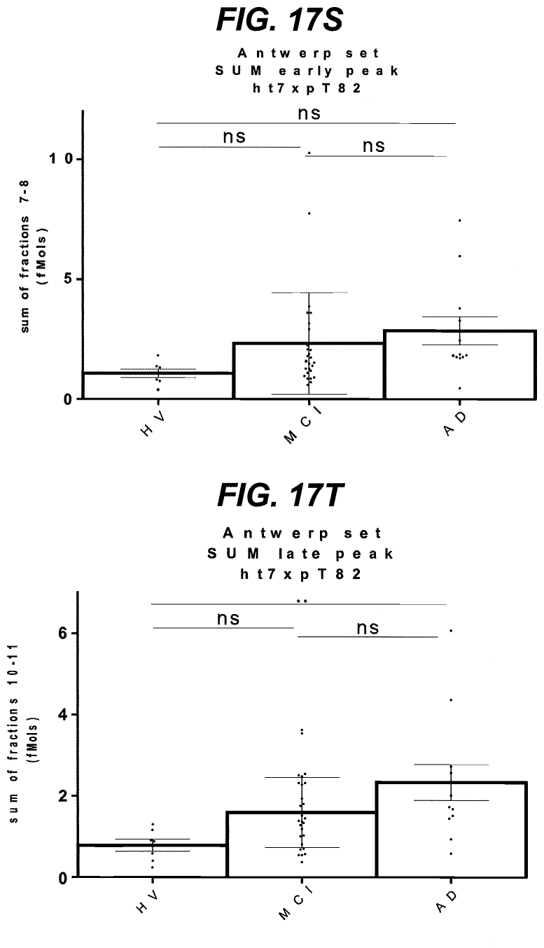

CSF samples were measured crude or after rpHPLC fractionation, using both pT3-based assays (pT3xhT43 & pT3xpT82) and tTau (hT7xpT82). All the pT3-based and hT7-based assays showed elevated signal progressively increasing signal in the HV vs MCI vs. AD groups (FIGS. 17A-C) and correlated well with each other (as seen Cohort 1, FIG. 9) (FIGS. 17D and 17E). The pT3 assays also correlated to some extent with Innotest tTau and pTau181 (FIGS. 17F and 17G), but not with Innotest AB42 or AB42/40 ration (FIGS. 17H and 17II). Similar results for diagnostic staging were observed in crude CSF measurements (FIGS. 17A-17C) or rpHPLC fractionated material (FIGS. 17J-17T). As seen in Cohort 3, the separation of HV vs MCI vs AD was more pronounced (greater statistically significance) using the pT3-based assays than the tTau assay, highlighting the pathological relevance of this pT3-assay measurement.

### Cohort 6 (“Disease Severity and Progression Cohort”)

CSF samples (LF) from clinically defined AD (Clinical Dementia Rating 1+) subjects (n=235) were obtained from Janssen study ELN115727301/302. These samples were baseline (pre-dose) samples from all subjects in the trial. In addition, CSF samples from 78 week follow up on the placebo subjects (n=90) was included to evaluate biomarkers of disease progression. Cognitive assessment (ADAS-COG, MMSE, NTB, and CDR.SOB), ApoE genotype, gender, & age were obtained from the trial. Innotest AB42, Innotest AB40, Simoa neurofilament light (NFL), pT3xpT82, pT3xhT43, and hT7xpT82 assays were performed at Janssen Neuroscience Biomarkers, La Jolla. Subjects were confirmed amyloid positive or negative based on the AB42/40 ratio cutoff of 0.09 (e.g. subjects with ratio<0.09=amyloid positive=AD, while those >0.09=amyloid negative=dementia from non-AD cause). 27 of the 235 subjects were determined to be amyloid negative thus each group was analyzed separately.

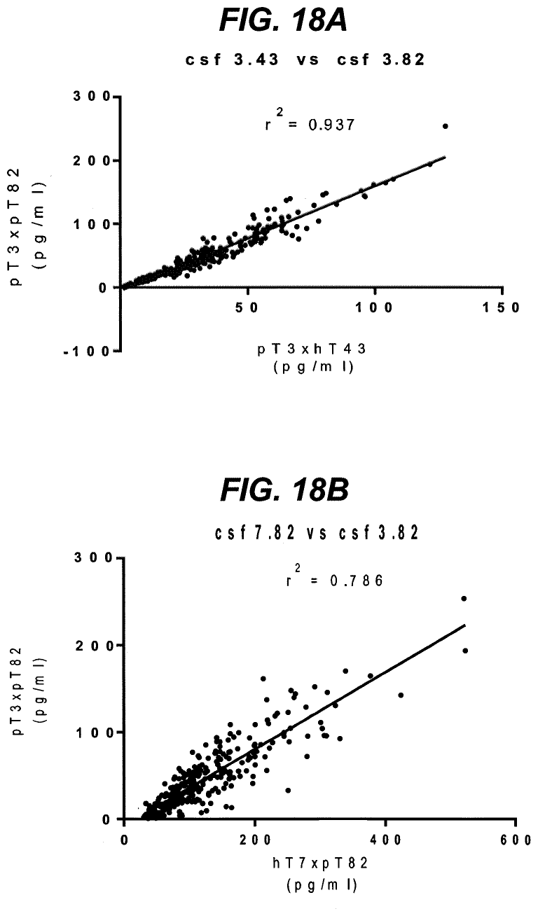

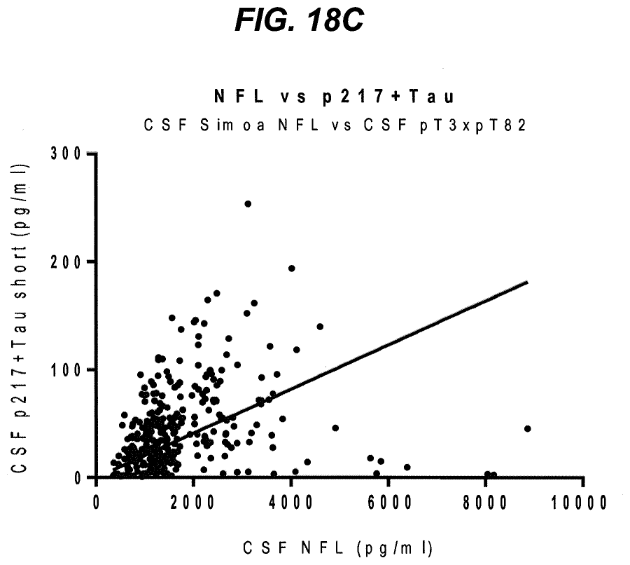

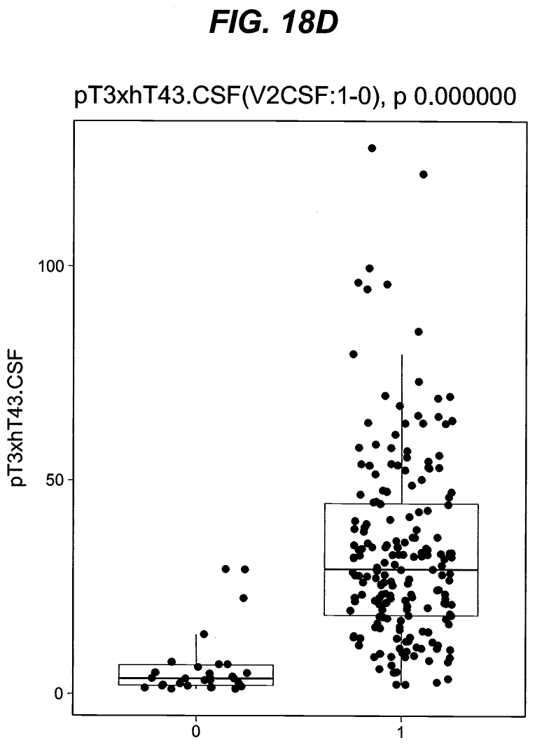

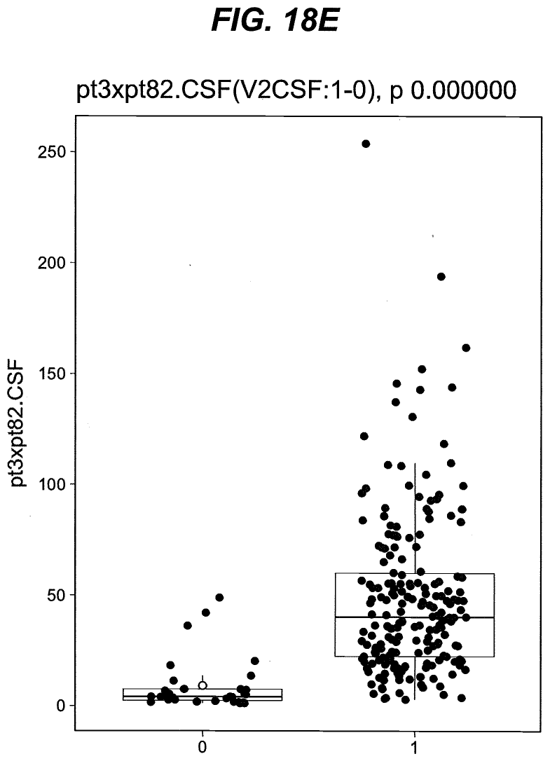

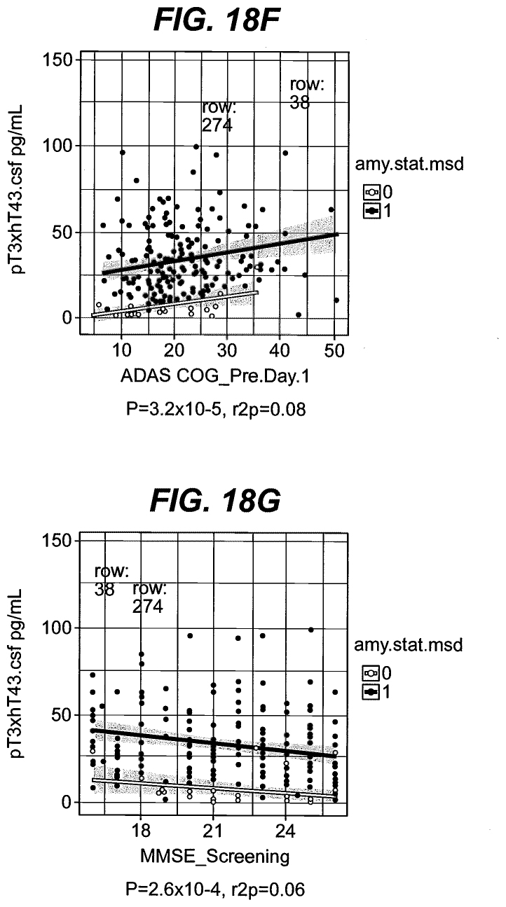

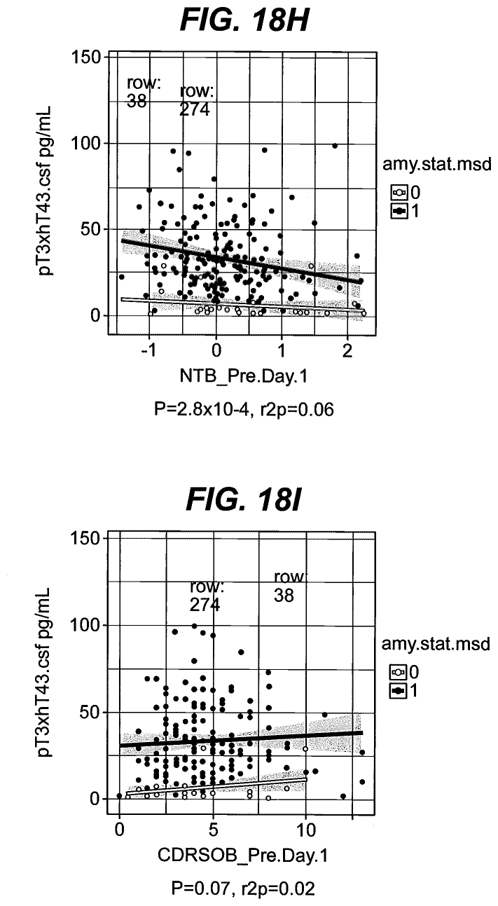

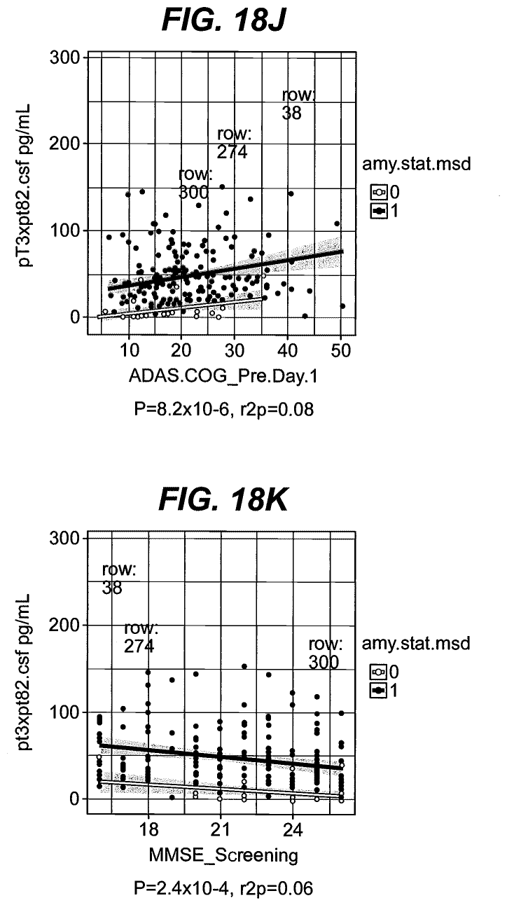

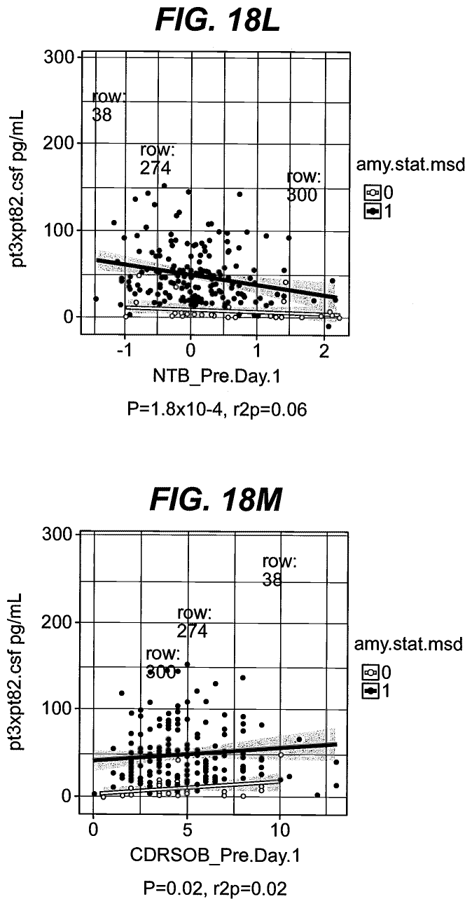

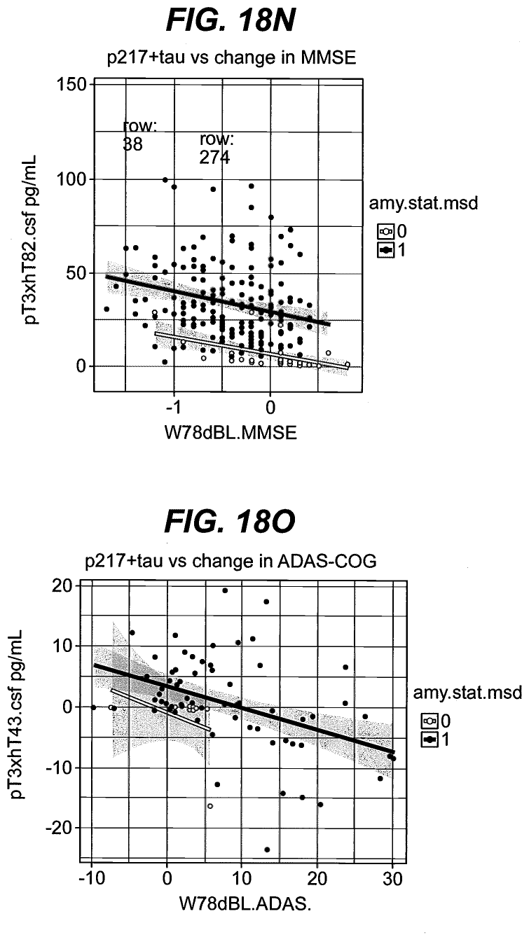

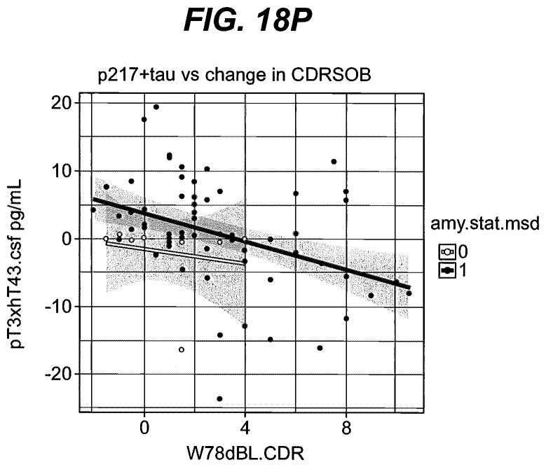

Signal from crude CSF measurements again revealed good correlation between the two pT3 assays and with the tTau assay (FIGS. 18A and 18B), but not with NFL (FIG. 18C), a suspected marker of general neurodegeneration, suggesting that the pT3-assay may recognize a specific form or stage of neurodegeneration.

The pT3-based assays again revealed higher signal in the amyloid positive vs negative subjects (FIGS. 18D-18E).

The pT3-based assays revealed modest correlation with several cognition scores (ADAS-COG, MMSE, NTB, CDR.SOB, FIGS. 18F-18M), corroborating the finding in Cohort 4 (FIG. 16B). Intriguingly the baseline pT3-based assay signal correlated modestly with change in cognition scores over the 18 month follow up period as well, suggesting ability to predict cognitive decline (FIGS. 18N-18P).

The ratio of pT3-based signal to tTau signal (p217_tau/tTau) yielded similar results, data not shown.

The correlations with cognition and change in cognition were seen in both the amyloid positive and negative groups, however the latter group was a small sample set. If confirmed this suggests the p217+vs cognition connection may not be AD-specific.

### Example 9. Quantification of p217+ Tau that is Free Vs. Bound by Antibody

The assays described in Example 3 were carried out as follows.

Assay 1: Quantification of Free Vs. Bound p217+ Tau in Biological Fluid Via Immunocapture Depletion Followed by rpHPLC

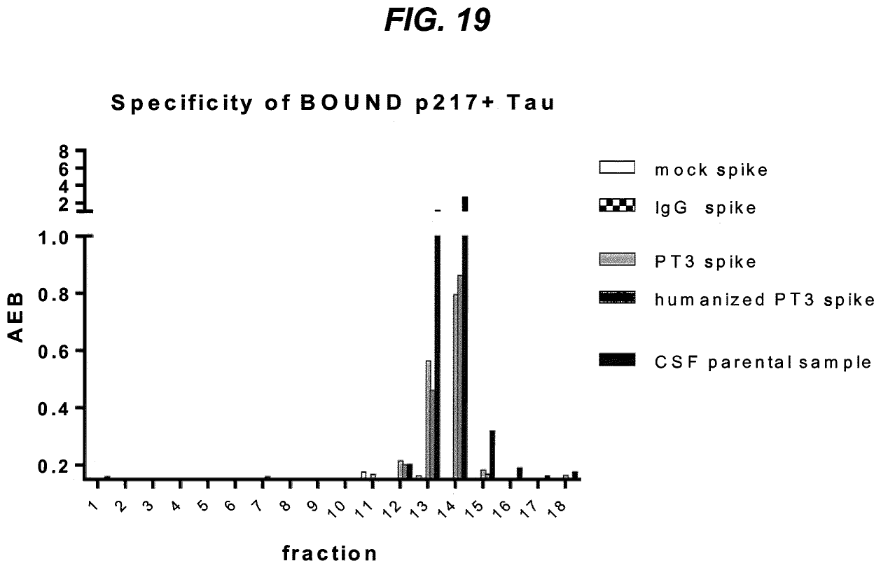

The assay was tested by spiking antibody into CSF samples. Pooled AD CSF was spiked with 10 ug of pT3 mAb, humanized pT3 mAb, msIgG or comparable volume of PBS (mock) and incubated at 4° C. for 24 hr followed by immunocapture. The samples, as well as parental CSF not subjected to immunocapture, were fractionated on rpHPLC, and each fraction was measured using the pT3xhT43 assay to assess the amount of total and bound p217+ tau. Substantial signal was observed in one major peak, similar to that seen in Example 7 and FIG. 7, in the parental sample (total p217+ tau) and the pT3 mAb or humanized pT3 mAb immunocaptures (bound p217+ tau), but not in the mock or IgG immunocaptures (FIG. 19).

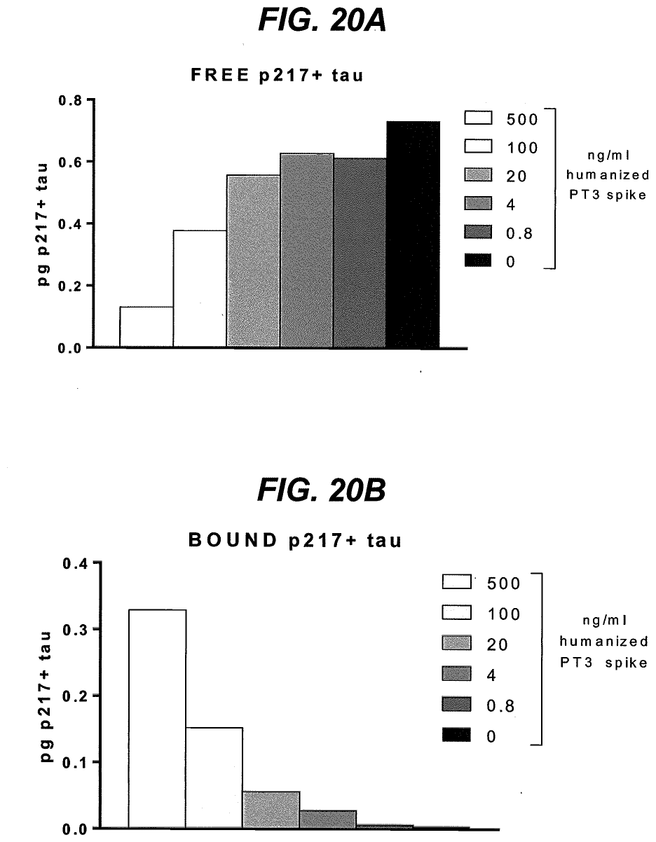

Pooled AD CSF was spiked with titrations of humanized pT3 mAb, incubated at 22° C. for 2 hr, followed by immunocapture, rpHPLC, and pT3xhT43 assay to assess bound p217+ tau (FIG. 20A). The IgG depleted supernatant was also fractionated and measured to assess free p217+ tau (FIG. 20B). Spiking with humanized pT3 mAb increased the amount of measured bound p217+ tau, and decreased the amount of free p217+ tau, in a dose dependent manner.

Taken together, the results show that this method, which is a direct measurement of target engagement, is specific for antibodies that are targeted to the p217+ tau epitope (FIG. 19) and is targeted-antibody dose dependent (FIG. 20).

Assay 2: Quantification of Free Vs. Bound p217+ Tau in Biological Fluid Via Selective Denaturing of Antibody

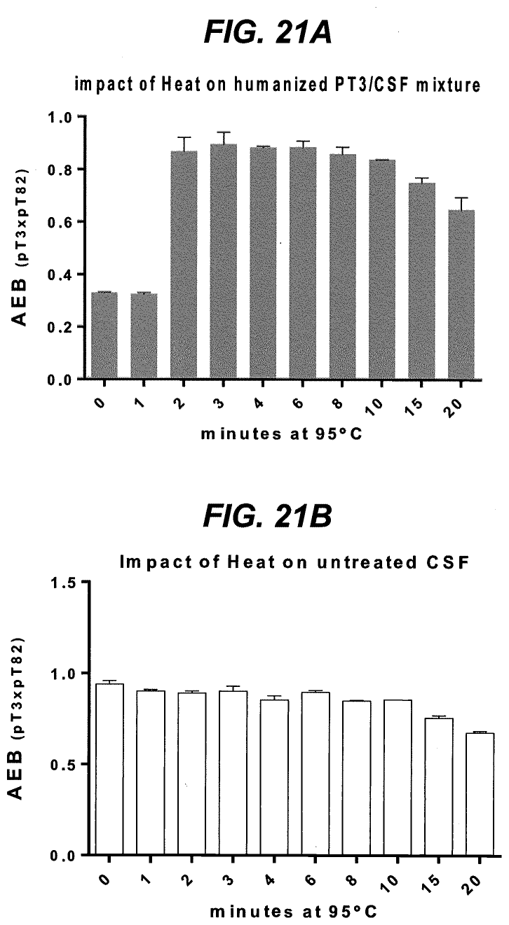

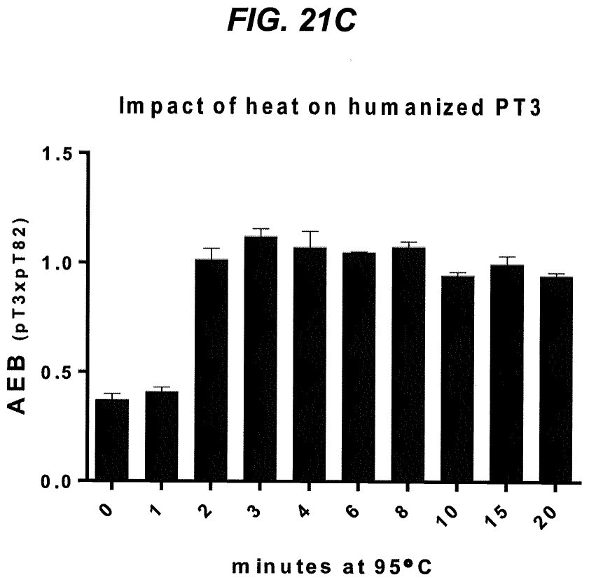

Biological samples (e.g. CSF) were heated at near boiling for 4 minutes, followed by a chilling on ice and subsequent measurement with the pT3xhT43 and/or pT3xpT82 assays. The precise time of this process were determined to irreversibly damage antibodies in the sample such that they cannot interfere with the assay (FIG. 21), but do not impact the p217+ tau signal itself (FIG. 21). It is believed that this is due to the particular lack of tertiary structure in tau protein, allowing it to be particularly stable at high temperature. This sample was termed total p217+ tau, while parallel measurement of a sample that had not been subjected to the heat treatment was termed free p217+ tau. Subtracting free from total concentration yielded bound p217+ tau measurement.

The impact of heat on the assay was determined, as follows.

Impact of heat on a CSF/humanized pT3 mAb mixture: Aliquots of pooled AD CSF were spiked with humanized pT3 mAb to 1 μg/ml, incubated for 2 hrs at 22° C., heated for 0-20 minutes at 95° C., chilled to 4° C., then measured using the pT3xpT82 assay at a 1:10 dilution (FIG. 21A). The p217+ tau signal was low through ˜2 minutes of heat treatment, then returned to levels seen in unspiked CSF and was stable through ˜10 minutes of heat before dropping.

Impact of heat of naïve CSF: Aliquots of pooled AD CSF were heated for 0-20 minutes at 95° C., then chilled to 4° C., before measuring using the pT3xpT82 assay at a 1:10 dilution (FIG. 21B). The p217+ tau signal was stable through ˜10 minutes of heat before dropping.

Impact of heat on ability of humanized pT3 mAb to interfere with the pT3xpT82 assay: Aliquots of humanized pT3 mAb at 10 μg/ml in PBS were heated for 0-20 minutes at 95° C., then chilled to 4° C. These samples were then mixed with pooled AD CSF (to 1 μg/ml final concentration of humanized pT3 mAb) and incubated for 2 hrs at 22° C. before measuring using the pT3xpT82 assay at a 1:10 dilution (FIG. 21C). The p217+ tau signal was low through ˜2 minutes of JNJ heat treatment, then returned to levels seen in unspiked CSF (see FIG. 21B) and was stable through at least 20 minutes of heat.

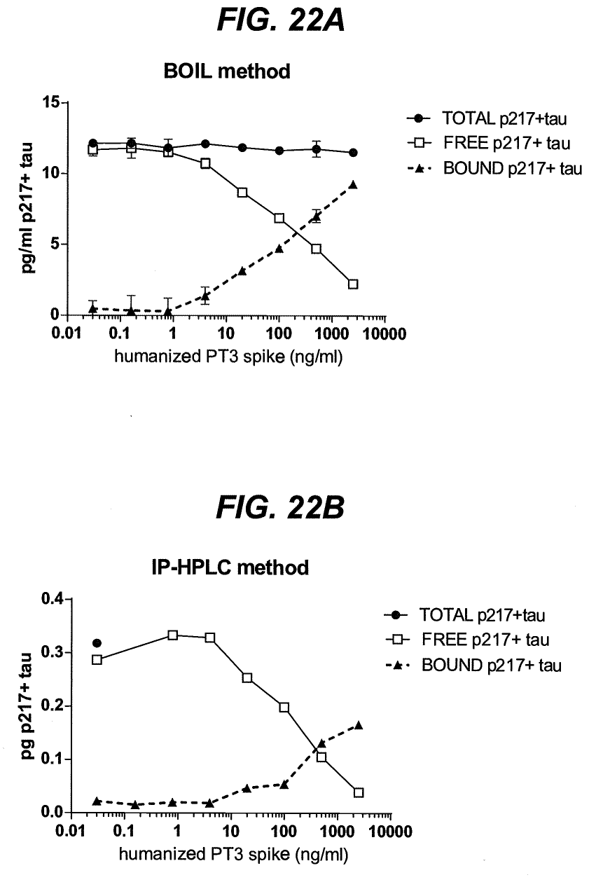

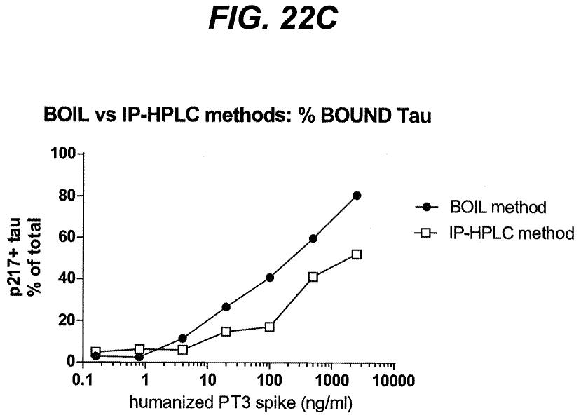

Parallel aliquots of pooled AD CSF were titrated with humanized pT3 mAb, incubated for 2 hr at 22° C., then subjected to either the heat denaturing process (with 4 minutes of heat) (FIG. 22A) or immunocapture/rpHPLC (FIG. 22B) before measurement using the pT3xpT82 assay. Both methods showed a humanized pT3 mAb dose dependent increase in bound, decrease in free, and no change in total p217+ tau signal. Further, the heat-mediated denaturing method yielded comparable humanized pT3 mAb dose dependency, and relative free vs. bound vs. total p217+ measurements, to that obtained using the more laborious immunocapture/rpHPLC method of Assay 1 (FIG. 22C). Therefore, the heat method is recommended for standard sample analysis.

### Example 10. P217+ Tau Signal in Preclinical Animal Models

To support preclinical studies, naïve samples from various common lab animals were evaluated using the pT3-based assays and/or sequence-aligned to predict cross reactivity.

### Cynomolgus Macaque

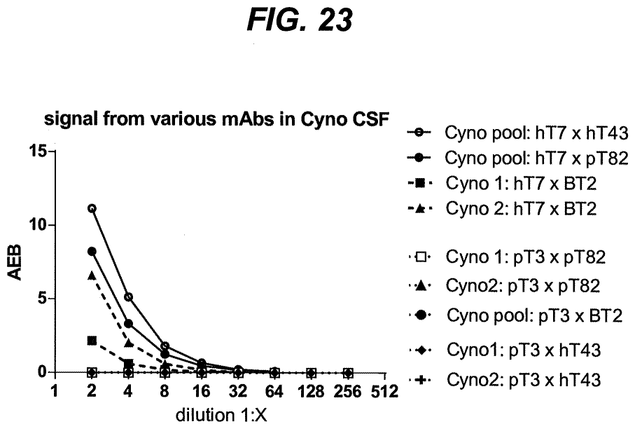

CSF from two Cynomolgus Macaques was measured at various dilutions using pT3-based and hT7-based assays (FIG. 23). For comparability, the same detection antibodies were paired with each of the two capture antibodies. In some cases, the two individual CSFs were tested separately (Cyno 1 or Cyno 2), and in other cases, the CSF samples were pooled to save volume. Substantial signal (AEB) was seen in all assays using hT7 as the capture antibody, regardless of the detection antibody, but no signal was detected in any of the assays using pT3 as the capture antibody. Additionally, plate based assays with pT3 have shown that even in homogenates of Cynomolgus Macaque brain, there is very little or no pT3-based signal, despite the large signal in AD human brain (data not shown). This suggests that despite high levels of tau, the pT3 epitope is not preserved in this species. Indeed, analysis of published protein sequences suggests one amino acid is different between human and Cynomolgus Macaque in the pT3 core epitope, and structural modeling, based on crystal structure of humanized pT3 mAb with tau, suggests this change could abolish binding of pT3 (data not shown).

### Common Marmoset

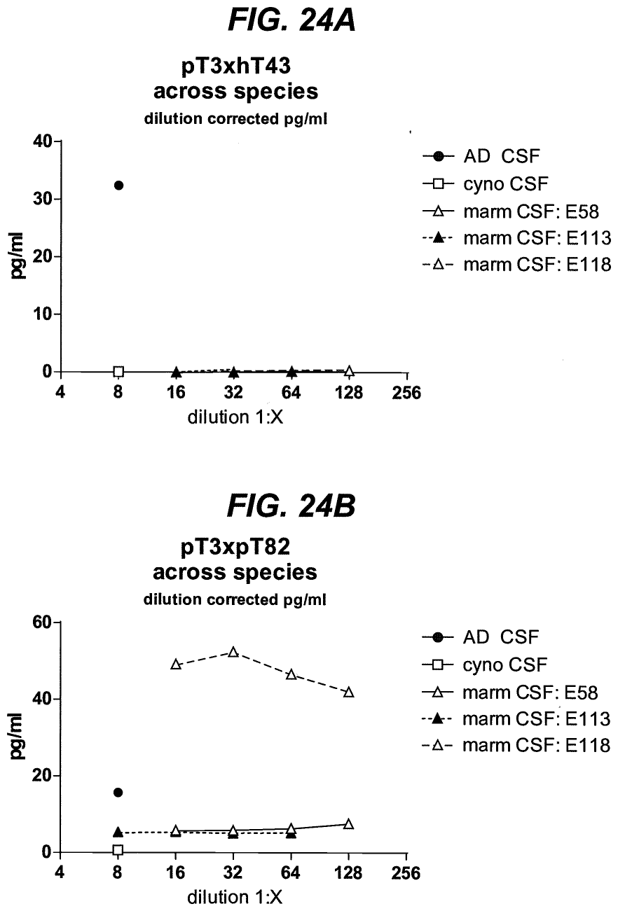

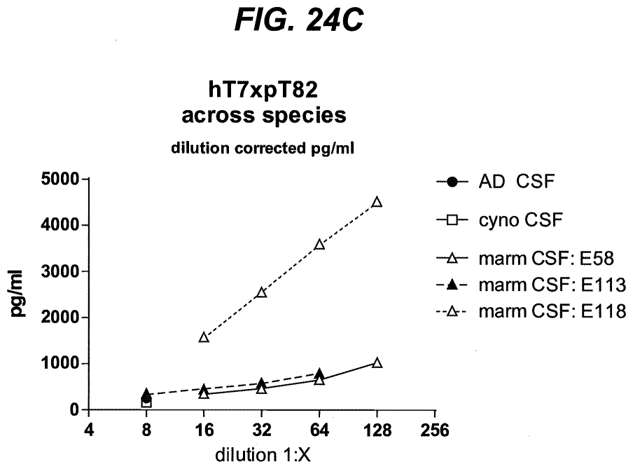

CSF from Common Marmoset was tested using pT3-based and hT7-based assays (FIG. 24). CSF from three Common Marmosets was measured at various dilutions using pT3xhT43, pT3xpT82, and hT7xpT82 assays. For comparability, a pooled Cynomolgus Macaque CSF (negative control) and pooled AD human CSF (positive control) were tested simultaneously. Substantial signal (AEB) was seen in Marmoset CSF using the pT3xpT82 (FIG. 24B) and hT7xpT82 (FIG. 24C) assays, but not with the pT3xhT43 assay (FIG. 24A).

This suggested that the hT43 epitope was lacking in this species, and indeed, a protein sequence alignment does indicate that one amino acid is different between human and Common Marmoset in the hT43 epitope, while the pT3, hT7, and pT82 epitopes are preserved. Measurement of Marmoset brain homogenate with same assays confirmed that there was substantial signal with the pT3xpT82 and hT7xpT82 assays, but very little with the pT3xhT43 assay (data not shown). Thus, analysis of p217+ tau signal in Marmoset was achieved using the pT3xpT82 assay.

### Mouse, Rat, Dog, Pig

Alignment of predicted tau protein sequences in mouse, rat, dog, or pig (NCBI Accession numbers: NP_001033698.1, NP_058908.2, NP_001104271.1, and AGJ26517.1, respectively) with human sequence suggests that pT3 is 100% conserved in these species. However, the hT43 and pT82 sequences of mouse, rat, dog and pig are not identical with those of human, and thus, samples from these would need to be evaluated using the pT3xhT43 and pT3xpT82 assays.

Taken together, the data presented here indicates that the pT3xhT43 and pT3xpT82 assays developed on the Simoa platform for CSF measurement are highly sensitive, having femtogram sensitivity, are precise, accurate, dilution linear, and analyte stable. The assays appear to correlate well with classical AD biomarkers and dementia scores and may be superior to those measures in identifying and staging AD subjects.

The assays can be used to measure the level of total p217+ tau in CSF, or to evaluate the fragment profile of p217+ in rpHPLC-fractionated CSF. The assays can also be combined with preanalytical manipulation to measure the levels of p217+ tau that is bound by endogenous or exogenously administered antibodies, vs. p217+ tau that is free of antibody. Thus, the assays can be used as predictive biomarkers to identify subjects for whom anti-p217+ tau antibody therapy will be suitable, by identifying subjects with high levels of the p217+ tau target. By measuring levels of total, free, and therapeutic antibody-bound p217+ tau, the assays can also be used as pharmacodynamics markers.

### Example 11. P217+ Tau Signal in Blood

While measurement of Tau in CSF has shown great utility in diagnosis and staging of neurodegenerative disorders, collection of CSF has limitations (e.g., patient burden, clinical site experience, collection volume and frequency constraints). As such, there is great interest in adapting Tau measurements to use on blood products (e.g. serum, plasma). Recent literature has however indicated that tau measurements in crude serum or plasma do not exhibit ideal diagnostic performance and may be plagued by sensitivity and matrix interference hurdles. The pT3-based assays may represent a new opportunity however due to its high sensitivity and specificity.

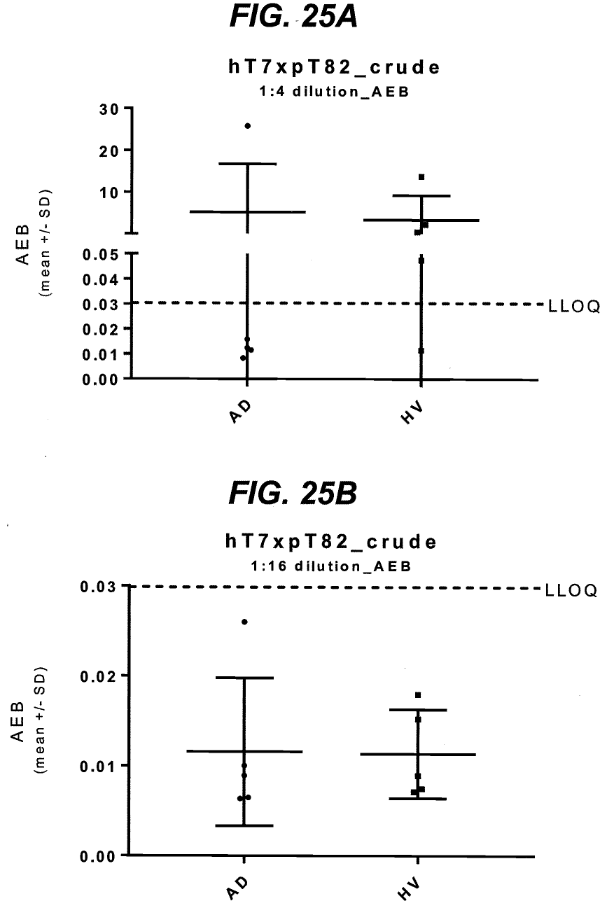

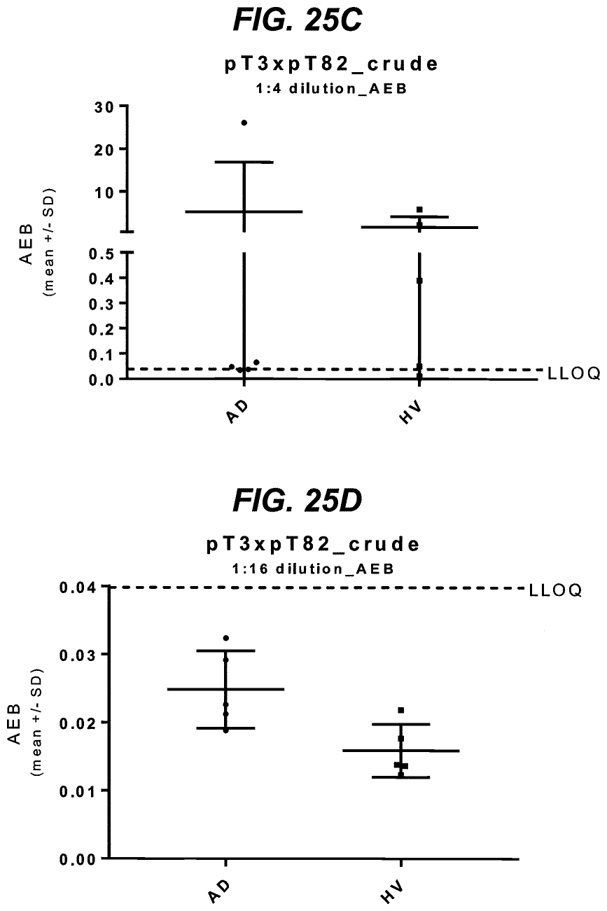

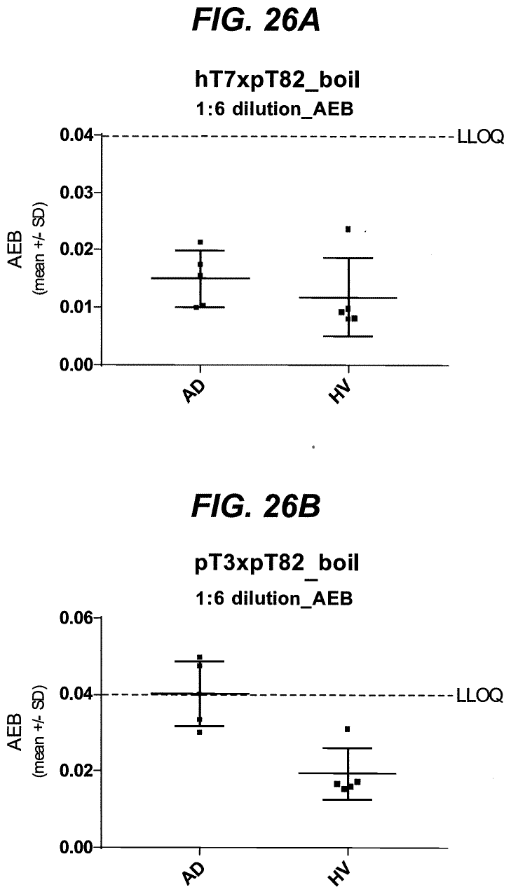

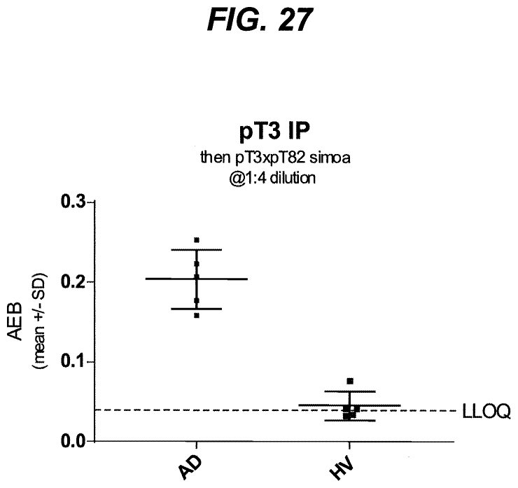

Serum from clinically defined AD & HV subjects (n=4 each) was measured with the pT3xpT82 and hT7xpT82 assays, either in crude sample at various dilutions (“crude”, FIGS. 25A-25D), in acid (NaOAc pH5) treated and denatured sample (“boil”, FIGS. 26A-26B) as in D'Abramo et al. 2016 to remove most matrix interference, and after immunoprecipitation (IP) with pT3 beads followed by heat denaturing of the elute (“pT3 IP”, FIG. 27).

Measurement in crude serum revealed most samples were below the limit of quantification (LOQ), with few outlier samples reporting much higher levels. However, the signal did not survive modest dilution and was deemed to thus be an interference artifact. Evaluation of the highest dilution tested (FIGS. 25B and 25D), and thus the least impacted by interreference, suggested that the pT3xpT82 assay may detect slightly more signal in AD samples, but all are below LOQ so may not be accurate and/or precise.

Measurement in serum after acid treatment (to dissociate protein-protein interactions) and heat (to denature most non-tau proteins) reduced all pT3xpT82 and hT7xpT82 signal to near or below LOQ (FIGS. 26A-26B). Again, the pT3xpT82 assay may detect slightly more signal in AD samples but all are near LOQ so may not be accurate and/or precise.

Measurement in serum after pT3-IP and denaturing, to remove most interfering substances and concentrate the p217+ tau, revealed much higher levels in the AD samples than in the HV samples (FIG. 27). The p217+ levels were ˜4× higher than in the crude or boil measurements and as such the HV samples were now at LOQ and the AD samples were now all in linear range.

These results indicated that the pT3-based assays described herein may have utility as a blood-based measurement of pathological tau, particularly when paired with and enrichment strategy such as IP.

While the invention has been described in detail, and with reference to specific embodiments thereof, it will be apparent to one of ordinary skill in the art that various changes and modifications can be made therein without departing from the spirit and scope of the invention.

### REFERENCES

Abhinandan and Martin, Mol Immunol. 45:3832-9, 2008
Almagro, Mol Recognit. 17:132-43, 2004
Barthelemy et al., J Alzheimers Dis. 51(4):1033-43, 2016
Butner and Kirschner, J Cell Biol. 115(3):717-30, 1991
Chothia and Lesk, J Mol Biol. 196:901-17, 1987
Chothia et al., J. Mol. Biol. 227: 799-817, 1992
Clavaguera et al., Nat Cell Biol. 11:909-13, 2009
D'Abramo et al Neurobiol Aging. 37:58-65, 2016
Epitope Mapping Protocols in Methods in Molecular Biology, Vol. 66, G. E. Morris, Ed. (1996)
Fishwild et al., Nat Biotechnol. 14:845-51, 1996
Frost et al., J Biol Chem. 284:12845-52, 2009
Hanger et al. J Biol Chem. 282(32):23645-54, 2007
Hanger et al., Trends Mol Med. 15:112-9, 2009
Iqbal et al., Curr Alzheimer Res. 7(8): 656-664, 2010
Knappik et al., J Mol Biol. 296:57-86, 2000
Kohler and Milstein, Nature. 256:495-7, 1975
Krebs et al., J Immunol Methods. 254:67-84, 2001
Lefranc et al., Dev Comp Immunol. 27:55-77, 2003
Lonberg et al., Nature. 368:856-9, 1994
Mendez et al., Nat Genet. 15:146-56, 1997
Meredith et al. PLoS One. 8(10):e76523, 2013
Morris et al., Neuron, 70:410-26, 2011
Russell et al., J Alzheimers Dis. 55(1):303-313, 2017
Shi et al., J Mol Biol. 397:385-96, 2010
Tramontano et al., J. Mol. Biol. 215:175-182, 1990
Wu and Kabat, J Exp Med. 132:211-50, 1970

1-21. (canceled)

22. A method of measuring p217+ tau peptides in a sample, comprising:
(a) contacting the sample with a capture antibody comprising an immunoglobulin heavy chain HCDR1, HCDR2 and HCDR3 comprising the polypeptide sequences of SEQ ID NOs: 32, 33 and 34, respectively and an immunoglobulin light chain LCDR1, LCDR2 and LCDR3 comprising the polypeptide sequences of SEQ ID NOs: 35, 36 and 37, respectively to capture the p217+ tau peptides in the sample, and
(b) contacting the captured p217+ tau peptides from (a) with at least one of a detection antibody selected from
(i) an antibody comprising an immunoglobulin heavy chain HCDR1, HCDR2 and HCDR3 comprising the polypeptide sequences of SEQ ID NOs: 12, 13 and 14, respectively and an immunoglobulin light chain LCDR1, LCDR2 and LCDR3 comprising the polypeptide sequences of SEQ ID NOs: 15, 16 and 17, respectively and
(ii) antibody comprising an immunoglobulin heavy chain HCDR1, HCDR2 and HCDR3 comprising the polypeptide sequences of SEQ ID NOs: 2, 3 and 4, respectively and an immunoglobulin light chain LCDR1, LCDR2 and LCDR3 comprising the polypeptide sequences of SEQ ID NOs: 5, 6 and 7, respectively
to thereby measure the p217+ tau peptides.

23. The method of claim 22, comprising contacting the captured p217+ tau peptides with both detection antibodies to thereby measure the amount of the p217+ tau peptides and an amount of long p217+ tau peptides, respectively, and determining a ratio of the amount of the long p217+ tau peptides to the amount of p217+ tau peptides.

24. The method of claim 23, further comprising
(i) determining an amount of short p217+ tau peptides via subtracting the amount of the long p217+ tau peptides from the amount of the p217+ tau peptides, and
(ii) determining a ratio of the amount of the short p217+ tau peptides to the amount of p217+ tau peptides, or a ratio of the amount of the long p217+ tau peptides to the amount of short p217+ tau peptides.

25. The method of claim 22, wherein the capture antibody is conjugated to a bead, and wherein the detection antibody is biotinylated.

26. The method of claim 22, wherein the lower limit of quantification of the method is about 40 fg/ml of the p217+ tau peptides and the lower limit of detection of the method is about 2 fg/ml of the p217+ tau peptides.

27. The method of claim 22, wherein the capture antibody comprises immunoglobulin heavy chain HCDR1, HCDR2 and HCDR3 having the polypeptide sequences of SEQ ID NOs: 32, 33 and 34, respectively, and immunoglobulin light chain LCDR1, LCDR2 and LCDR3 having the polypeptide sequences of SEQ ID NOs: 35, 36 and 37, respectively.

28. The method of claim 22, wherein the antibody of (b)(i) comprises immunoglobulin heavy chain HCDR1, HCDR2 and HCDR3 having the polypeptide sequences of SEQ ID NOs: 2, 3 and 4, respectively; and immunoglobulin light chain LCDR1, LCDR2 and LCDR3 having the polypeptide sequences of SEQ ID NOs: 5, 6 and 7, respectively.

29. The method of claim 22, wherein the antibody of (b)(ii) comprises immunoglobulin heavy chain HCDR1, HCDR2 and HCDR3 having the polypeptide sequences of SEQ ID NOs: 12, 13 and 14, respectively; and immunoglobulin light chain LCDR1, LCDR2 and LCDR3 having the polypeptide sequences of SEQ ID NOs: 15, 16 and 17, respectively.

30. The method of claim 22, wherein the sample is a biological sample from a subject selected from the group consisting of blood, brain homogenate, and cerebral spinal fluid (CSF) from the subject.

31. The method of claim 30 wherein the biological sample is blood.

32. The method of claim 30 wherein the biological sample is CSF.

33. The method of claim 30 further comprising fractionating the biological sample using reverse phase high-performance liquid chromatography (rpHPLC) prior to contacting the sample with the capture antibody.

34. The method of claim 30, further comprising determining if the subject suffers from a tauopathy or is at risk of developing a tauopathy comprising comparing at least one of:
a) the amount of the p217+ tau peptides,
b) an amount of the long p217+ tau peptides, and
c) an amount of the short p217+ tau peptides determined via subtracting the amount of the long p217+ tau peptides from the amount of the p217+ tau peptides,
from the subject with a corresponding baseline value wherein an increase in the amount as compared to the baseline indicates the subject suffers from or is at risk from suffering from a tauopathy.

35. The method of claim 34, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

36. The method of claim 35, wherein the tauopathy is Alzheimer's disease.

37. The method of claim 35, wherein the tauopathy is progressive supranuclear palsy.

38. The method of claim 30, further comprising determining the effectiveness of a treatment of a tauopathy in the subject comprising comparing at least one of:
a) the amount of the p217+ tau peptides,
b) an amount of the long p217+ tau peptides, and
c) an amount of the short p217+ tau peptides determined via subtracting the amount of the long p217+ tau peptides from the amount of the p217+ tau peptides,
from the subject with a corresponding baseline value wherein a decrease in the amount as compared to the baseline indicates the treatment is effective.

39. The method of claim 38, wherein the tauopathy is selected from the group consisting of familial Alzheimer's disease, sporadic Alzheimer's disease, frontotemporal dementia with parkinsonism linked to chromosome 17 (FTDP-17), progressive supranuclear palsy, corticobasal degeneration, Pick's disease, progressive subcortical gliosis, tangle only dementia, diffuse neurofibrillary tangles with calcification, argyrophilic grain dementia, amyotrophic lateral sclerosis parkinsonism-dementia complex, Down syndrome, Gerstmann-Straussler-Scheinker disease, Hallervorden-Spatz disease, inclusion body myositis, Creutzfeld-Jakob disease, multiple system atrophy, Niemann-Pick disease type C, prion protein cerebral amyloid angiopathy, subacute sclerosing panencephalitis, myotonic dystrophy, non-Guamanian motor neuron disease with neurofibrillary tangles, postencephalitic parkinsonism, chronic traumatic encephalopathy, and dementia pugulistica (boxing disease).

40. The method of claim 39, wherein the tauopathy is Alzheimer's disease.

41. The method of claim 39, wherein the tauopathy is progressive supranuclear palsy.
